(19) United States
(12) Patent Application Publication
(10) Pub. No.: US 2026/0232670 A1
(43) Pub. Date: Aug. 13, 2026

(54) TYRO3 SELECTIVE INHIBITORS AND USES THEREOF

(71) Applicant: THE UNIVERSITY OF NORTH CAROLINA AT CHAPEL HILL, Chapel Hill, NC (US)

(72) Inventors: Xiaodong WANG, Chapel Hill, NC (US); Deyu KONG, Chapel Hill, NC (US); Xiangbo YANG, Chapel Hill, NC (US)

(21) Appl. No.: 19/151,175

(22) PCT Filed: Jan. 26, 2024
(86) PCT No.: PCT/US2024/013109

§ 371 Date: Jul. 25, 2025

Related U.S. Application Data
(60) Provisional application No. 63/481,893, filed on Jan. 27, 2023.

(51) Int. Cl. A61K 31/519; A61K 31/53; A61K 31/5377; A61K 31/541; A61K 31/551; C07D 487/04

(52) U.S. Cl. CPC A61K 31/519; A61K 31/53; A61K 31/5377; A61K 31/541; A61K 31/551; C07D 487/04

(57) ABSTRACT

The subject matter described herein is directed to compounds of Formula A; and pharmaceutically acceptable salts thereof, and compositions thereof, including pharmaceutical compositions, and methods of treating disorders associated with TAM receptor tyrosine kinases and/or TYRO3.

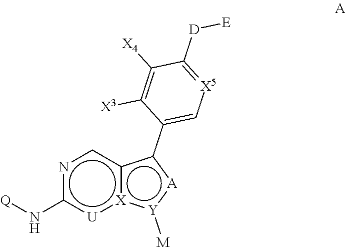

### CROSS-REFERENCE

This application claims the benefit of U.S. Provisional Application No. 63/481,893, filed Jan. 27, 2023, which is incorporated herein by reference it its entirety.

### STATEMENT REGARDING FEDERALLY SPONSORED RESEARCH OR DEVELOPMENT

This invention was made with government support under CA259077 awarded by the National Institute of Health. The government has certain rights in this invention.

### BACKGROUND

The TAM-family (TYRO3, AXL, and MER) tyrosine kinases are expressed on macrophages, dendritic cells, monocyte-derived suppressor cells (MDSCs) and other innate immune cells. These receptors are activated by ligands complexed with phosphatidylserine (PtdSer) on the surface of apoptotic and virus-infected cells. In macrophages, ligand binding triggers apoptotic cell engulfment and activates expression of an anti-inflammatory cytokine profile and immune checkpoint pathway components, leading to immune suppression.

Cancer cells hijack this system to suppress anti-tumor immunity and thereby promote tumor growth and progression. Conversely, TAM kinase inhibitors can promote anti-tumor immunity and studies demonstrating the potential of TAM kinases as immuno-oncology targets have been increasingly reported in recent years. First-in-class MERTK or AXL-selective inhibitors have been developed and advanced to early-stage clinical trials; however, selective TYRO3 inhibitors with properties suitable for clinical application have not been described.

Availability of selective TYRO3 inhibitors would provide flexibility to combine with other TAM kinase inhibitors or to target TYRO3 alone. The subject matter described herein addresses this need.

### BRIEF SUMMARY

In certain embodiments, the subject matter described herein is directed to compounds of Formula A, which includes all sub-formulae of Formula A, such as Formula I and all sub-formulae of Formula I:

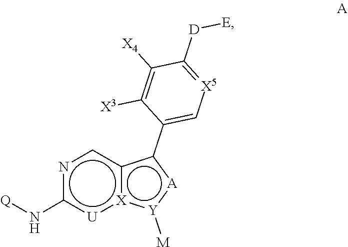

wherein:
U is N or CH; X is N or C; Y is N or C, and A is N or CH, wherein two or three of U, X, Y and A is N, and at least one of U and X is N;
D is selected from the group consisting of a bond, (CH₂)ₘ, (CD₂)ₘ, (CHD)ₘ, (CF₂)ₘ, (CHF)ₘ, —O—, —CO—, —N(H)— and —S(O₂)—;
E is selected from the group consisting of:
   —NR^{E1}R^{E2}, wherein REI is hydrogen, deuterium or C₁-C₆ alkyl, and R^{E2} is substituted or unsubstituted C₁-C₆ alkyl; and,

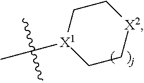

wherein:
X¹ is CH or N;
X² is CH₂, O, S, or N—R⁶, wherein R⁶ is hydrogen, substituted or unsubstituted C₁-C₆ alkyl, or C₃-C₉ cycloalkyl;
X³ and X⁴ are independently selected from hydrogen and halogen;
X⁵ is CH or N;
Q is selected from the group consisting of:

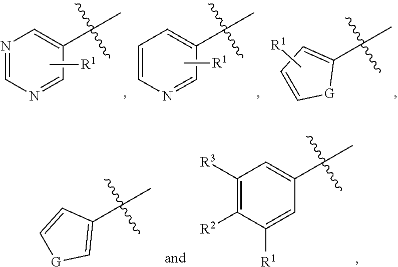

wherein, G is S, O or N;
M is

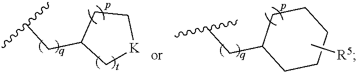

K, if present, is O or CR⁷R⁸, wherein R⁷ and R⁸ are independently selected from C₁-C₆ alkylhydroxy, or R⁷ and R⁸ together with the carbon to which each is attached form a carbonyl or a 5-6 membered heterocycloalkyl (spiro);
m is 0, 1, or 2;
j is 0, 1, 2, or 3;
p is 0 or 1;
q is 0 or 1;
t is 1 or 2;
R¹ is selected from the group consisting of halogen, C₁-C₆ alkyl, C₁-C₆ alkoxy, hydroxy, nitro, C₁-C₆-haloalkyl, cyano, and N(R^{4a})₂,
   wherein each R^{4a} is independently selected from the group consisting of hydrogen, and C₁-C₆-alkyl;
R² and R³ are each independently selected from the group consisting of hydrogen, halogen, deuterium, C₁-C₆ alkyl, C₁-C₆ alkoxy, hydroxy, nitro, C₁-C₆-haloalkyl, cyano, and N(R^{4b})₂,
   wherein each R^{4b} is independently selected from the group consisting of hydrogen and C₁-C₆-alkyl;
      or,
R¹ and R² or R² and R³ together with the carbon to which each is attached form a ring selected from the group consisting of substituted or unsubstituted C₃-C₈ cycloalkyl, substituted or unsubstituted C₃-C₈ heterocycloalkyl, substituted or unsubstituted C₃-C₈ aryl, and substituted or unsubstituted C₃-C₈ heteroaryl, wherein the heteroatom is O, N, or S; and,
R⁵ is selected from the group consisting of hydroxy, cyano, halogen, amino, C₁-C₆ alkyl, C₁-C₆ alkoxy, mono(C₁-C₆)alkylamino, di(C₁-C₆)alkylamino, N-alkoxycarbonyl and C₁-C₆ alkylhydroxy;
   or a pharmaceutically acceptable salt thereof.

In certain embodiments, the subject matter described herein is directed to pharmaceutical compositions comprising a therapeutically effective amount of a compound of Formula A, which includes all sub-formulae of Formula A, such as Formula I and all sub-formulae of Formula I, and a pharmaceutically acceptable carrier.

In certain embodiments, the subject matter described herein is directed to methods for the treatment of disorders associated with TAM receptor tyrosine kinases, the method comprising the step of administering to the subject an effective amount of at least one compound of Formula A, which includes all sub-formulae of Formula A, such as Formula I and all sub-formulae of Formula I.

In certain embodiments, the subject matter described herein is directed to methods of treatment of disorders associated with TYRO3 tyrosine kinases, the method comprising the step of administering to the subject an effective amount of at least one compound of Formula A, which includes all sub-formulae of Formula A, such as Formula I and all sub-formulae of Formula I.

In certain embodiments, the subject matter described herein is directed to methods of inhibiting TAM receptor tyrosine kinases, in at least one cell, the method comprising the step of contacting the at least one cell with an effective amount of at least one compound of Formula A, which includes all sub-formulae of Formula A, such as Formula I and all sub-formulae of Formula I.

In certain embodiments, the subject matter described herein is directed to methods of inhibiting TYRO3 tyrosine kinases, in at least one cell, the method comprising the step of contacting the at least one cell with an effective amount of at least one compound of Formula A, which includes all sub-formulae of Formula A, such as Formula I and all sub-formulae of Formula I.

### DETAILED DESCRIPTION

Development of agents with selectivity for TYRO3, particularly relative to the other TAM kinases, is desirable. Described herein are compounds of Formula A and their uses for the treatment of disorders associated with TAM receptor tyrosine kinases. Also described herein are compounds of Formula A to inhibit TAM receptor tyrosine kinases. Described herein are compounds of Formula A and their uses for the treatment of disorders associated with TYRO3 receptor tyrosine kinases. Also described herein are compounds of Formula A to inhibit TYRO3 receptor tyrosine kinases.

Described herein are compounds of Formula A that are highly selective for inhibition of TYRO3 tyrosine kinases. In certain embodiments, the compounds of Formula A described herein inhibit TYRO3 tyrosine kinases at concentrations of less than 10 nM. In certain embodiments, the compounds of Formula A described herein inhibit TYRO3 tyrosine kinases at concentrations of less than 5 nM.

The presently disclosed subject matter will now be described more fully hereinafter. However, many modifications and other embodiments of the presently disclosed subject matter set forth herein will come to mind to one skilled in the art to which the presently disclosed subject matter pertains having the benefit of the teachings presented herein. Therefore, it is to be understood that the presently disclosed subject matter is not to be limited to the specific embodiments disclosed and that modifications and other embodiments are intended to be included within the scope of the appended claims. In other words, the subject matter described herein covers all alternatives, modifications, and equivalents. In the event that one or more of the incorporated literature, patents, and similar materials differs from or contradicts this application, including but not limited to defined terms, term usage, described techniques, or the like, this application controls. Unless otherwise defined, all technical and scientific terms used herein have the same meaning as commonly understood by one of ordinary skill in this field. All publications, patent applications, patents, and other references mentioned herein are incorporated by reference in their entirety.

### I. Overview

The compounds described herein exhibit advantageous selectivity among the TAM family, such as above 6-fold over MERTK, and about 19-fold over AXL. The compounds described herein are particularly useful in treating thrombosis, and related conditions.

As mentioned above, while most studies have focused on MERTK and/or AXL, numerous studies have demonstrated roles for TYRO3 in suppression of anti-tumor immunity, best characterized in melanoma models. Like the other TAM kinases, TYRO3 can stimulate expression of Suppressor of Cytokine Signaling (SOCS) proteins, leading to induction of the immune checkpoint ligand PD-L1, decreased JAK-STAT signaling, and induction of an anti-inflammatory cytokine profile.¹⁻³ Melanoma cells can produce PROS1 ligand, which stimulates TYRO3 on macrophages and thereby inhibits expression of inflammatory M1 cytokines.² Similarly, MDSCs were decreased in melanomas from Tyro3−/− mice relative to wild-type mice, and had reduced STAT3 activity and immuno-suppressive function.⁴ Moreover, co-implantation of Tyro3−/− MDSCs with tumor cells reduced tumor growth compared to wild-type MDSCs. These observations validate TYRO3 as a potential therapeutic target in the tumor microenvironment.

TYRO3 is also aberrantly expressed in a wide variety of different malignancies, including breast, prostate, ovarian, lung, colorectal, liver, skin, bladder and hematologic cancers.⁵⁻⁶ In this context, TYRO3 functions to promote tumor cell survival and oncogenic phenotypes. The dual roles for TYRO3 and the other TAM kinases in tumor cells and the immune system suggest that agents targeting these proteins will provide both direct tumor cell killing and immune-mediated anti-tumor activities, making them particularly attractive therapeutic targets. Regulation of bone homeostasis by MerTK and Tyro3 ascribes their potential use as an osteoanabolic therapy with implications in cancer.²⁸

In addition, TYRO3 plays a critical role in platelet aggregation as a platelet response amplifier. Thrombosis is an occlusive blood clot that forms in an artery or a vein and is the common pathology of ischemic heart disease, stroke, and acute myocardial infarction.⁷ Blood must remain fluid within the vasculature. However, when injury to a blood vessel happens, a hemostasis process via platelet deposition and the coagulation cascade is initiated to prevent blood loss.⁸ The consequence of this process is the formation of a fibrin clot.⁹ On the other hand, when a clot is formed within a blood vessel, the fibrinolytic system is rapidly activated to dissolve the thrombus to restore the fluidity of blood.¹⁰ In healthy states, complex interactions between the coagulation and anticoagulation systems maintain the balance between clotting and bleeding. However, this natural balance is interrupted whenever the procoagulant activity of the coagulation factors is increased, which may result in bleeding or thrombotic diseases.¹¹

Gas6 (arrest-specific gene 6) and protein S are TYRO3 biological ligands and structurally belong to the family of plasma vitamin K-dependent proteins which participate in platelet thrombus formation.¹² Existing evidence suggests that TYRO3 antibodies displayed potent inhibition of platelet aggregation in vitro as well as comparable antithrombotic efficacy in a rat ferric chloride model compared to clopidogrel which is used as standard treatment with acetylsalicylic acid for thromboembolic disorders.²⁹ Thromboembolic disorders, such as acute myocardial infarction and stroke, are still the most common causes of mortality and morbidity in developed countries, most of which are the consequence of acute arterial thrombosis.¹³ In patients with arterial thromboembolic disorders, the current gold standard treatment is dual antiplatelet therapy with acetylsalicylic acid and thienopyridine such as clopidogrel. However, clopidogrel is a thienopyridine prodrug that requires metabolic conversion by the cytochrome P450 CYP2C19 pathway to generate the active metabolite that irreversibly binds to the P2Y₁₂ receptor, resulting in a slow onset and a high risk for increased bleeding.¹⁴⁻¹⁶ In addition, about 30% of the patients are resistant to clopidogrel treatment due to CYP2C19 gene polymorphisms.¹⁷ Consequently, an effective improvement strategy in efficacy while maintaining a better safety profile is still actively searched.

Besides antithrombotic efficacy mentioned above, TYRO3 antibodies also showed a safety profile superior to what was observed for clopidogrel in a rat tail-bleeding model.¹⁸⁻²¹ Moreover, when the TYRO3 receptor was knockout in mice, initial platelet aggregation occurred, but platelet aggregates stabilization was impaired, and thrombosis formation was blocked.²² This inhibition prevented thrombus but did not increase bleeding, making TYRO3 inhibition a therapeutic option in thrombotic disorders. Thus, TYRO3 inhibitors also have the potential to provide therapeutic benefits without increasing the bleeding risk.

Of note, due to the overlapping functions shared by the TAM kinases, the phenotypes associated with TAM kinase inhibition become progressively more pronounced in mice lacking multiple family members. Thus, approaches that rely on sustained inhibition of all 3 TAM kinases can have increased side effects relative to more selective TAM kinase inhibitors. At least for these reasons, development of agents with selectivity for TYRO3, particularly relative to the other TAM kinases, is desirable. There are known multi-kinase inhibitors with activity against TYRO3, such as BMS-77760723 and RXDX-10624-25, however, their intended targets are other oncogenic kinases and/or they target multiple TAM kinases.

As also noted above, compounds of Formula A described herein have anti-platelet activity. Platelets are small cells derived from precursor megakaryocytes. The physiologic procoagulant activity of platelets helps prevent excessive bleeding, while increased platelet activation and overactive coagulation can lead to pathologic thrombus formation which may result in stroke or heart attack. Anti-platelet compounds are therefore an important family of drugs for cardiovascular diseases and for certain surgical procedures where a risk of stroke or thrombosis is prevalent. However, current anti-platelet therapies are often complicated by significant bleeding side effects. For example, aspirin, an antiplatelet drug that functions through inhibiting the production of thromboxane, has a baseline major bleeding risk (gastrointestinal or intracranial) of 1-4%. The addition of clopidogrel (Plavix), another widely used antiplatelet drug, as a combination therapy further increases that risk by 1%. Moreover, not all patients respond to aspirin and clopidogrel: the non-response rates are 5.5-60% in patients treated with aspirin and 4-30% in those treated with clopidogrel based on meta-analyses. Consequently, there is still a significant need to develop therapies for treatment of thrombosis, especially ones that do not increase the risk for bleeding.

Mer is a member of the TAM (Tyro3, Axl and Mer) receptor tyrosine kinase (RTK) subfamily with growth-arrest-specific-6 (Gas6) as one of the endogenous ligands. Elevated Mer activation has been strongly associated with the oncogenesis of a number of human cancers. Recently, Mer has also been shown to play important roles in regulating macrophage activity and platelet aggregation. Mer knock-out mice have decreased platelet aggregation while maintaining normal bleeding times and coagulation parameters. Consequently, these mice are protected from thrombosis without increased spontaneous bleeding. These observations indicate that small molecule Mer kinase inhibitors are active as anti-platelet drugs with decreased bleeding complications, a profile that confers a major advantage over currently available anti-platelet therapies. Therefore, TYRO3 and MERTK dual inhibitors also have the potential to provide therapeutic benefits without increasing the bleeding risk.

### II. Definitions

As used herein, “Formula A” includes all sub-formulae of Formula A described herein, including Formula I.

As used herein, “Formula I,” includes all sub-formulae of Formula I described herein.

“Alkyl” as used herein alone or as part of another group, refers to a straight or branched chain hydrocarbon containing from 1 to 10 carbon atoms. Alkyls can be deuterated. Representative examples of alkyl include, but are not limited to, methyl, CD₃, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, and the like. The term “alkyl” is intended to include both substituted and unsubstituted alkyl unless otherwise indicated and these groups may be substituted with groups selected from halo (e.g., haloalkyl), alkyl, haloalkyl, alkenyl, alkynyl, cycloalkyl (including spiroalkyl, e.g., C2, C3, or C4 spiroalkyl), cycloalkylalkyl, aryl, arylalkyl, heterocyclo, heterocycloalkyl, hydroxyl, alkoxy (thereby creating a polyalkoxy such as polyethylene glycol), alkenyloxy, alkynyloxy, haloalkoxy, cycloalkoxy, cycloalkylalkyloxy, aryloxy, arylalkyloxy, heterocyclooxy, heterocyclolalkyloxy, mercapto, amino, carboxy, alkylamino, alkenylamino, alkynylamino, haloalkylamino, cycloalkylamino, cycloalkylalkylamino, arylamino, arylalkylamino, heterocycloamino, heterocycloalkylamino, disubstituted-amino, ester, amide, sulfonamide, nitro or cyano.

“Cycloalkyl” as used herein alone or as part of another group, refers to a saturated or partially unsaturated cyclic hydrocarbon group containing from 3, 4 or 5 to 6, 7 or 8 carbons (which carbons may be replaced in a heterocyclic group as discussed below) and includes spirocyclics. Representative examples of cycloalkyl include, cyclopropyl, cyclobutyl, cyclopentyl, cyclohexyl, cycloheptyl, and cyclooctyl. These rings may be optionally substituted with additional substituents as described herein such as halo or loweralkyl. The term “cycloalkyl” is generic and intended to include heterocyclic groups as discussed below unless specified otherwise.

“Heterocyclic group” or “heterocycloalkyl” as used herein alone or as part of another group, refers to an aliphatic (e.g., fully or partially saturated heterocycloalkyl) or aromatic (e.g., heteroaryl) monocyclic- or a bicyclic-ring system. Monocyclic ring systems are exemplified by any 5- or 6-membered ring containing 1, 2, 3, or 4 heteroatoms independently selected from oxygen, nitrogen, and sulfur. The 5 membered ring has from 0-2 double bonds and the 6 membered ring has from 0-3 double bonds. A heterocycloalkyl may be a single ring or multiple rings wherein the multiple rings may be fused, bridged or spiro, and may comprise one or more (e.g., 1 to 3) oxo (═O) or N-oxide (N—O⁻) moieties. Representative examples of monocyclic ring systems that are heterocycloalkyls include, but are not limited to, azetidine, azepine, aziridine, diazepine, 1,3-dioxolane, dioxane, dithiane, furan, imidazole, imidazoline, imidazolidine, isothiazole, isothiazoline, isothiazolidine, isoxazole, isoxazoline, isoxazolidine, morpholine, oxadiazole, oxadiazoline, oxadiazolidine, oxazole, oxazoline, oxazolidine, piperazine, piperidine, pyran, pyrazine, pyrazole, pyrazoline, pyrazolidine, pyridine, pyrimidine, pyridazine, pyrrole, pyrroline, pyrrolidine, tetrahydrofuran, tetrahydrothiophene, tetrazine, tetrazole, thiadiazole, thiadiazoline, thiadiazolidine, thiazole, thiazoline, thiazolidine, thiophene, thiomorpholine, thiomorpholine sulfone, thiopyran, triazine, triazole, trithiane, and the like. Bicyclic ring systems are exemplified by any of the above monocyclic ring systems fused to an aryl group as defined herein, a cycloalkyl group as defined herein, or another monocyclic ring system as defined herein. Representative examples of bicyclic ring systems include but are not limited to, for example, benzimidazole, benzothiazole, benzothiadiazole, benzothiophene, benzoxadiazole, benzoxazole, benzofuran, benzopyran, benzothiopyran, benzodioxine, 1,3-benzodioxole, cinnoline, indazole, indole, indoline, indolizine, naphthyridine, isobenzofuran, isobenzothiophene, isoindole, isoindoline, isoquinoline, phthalazine, purine, pyranopyridine, quinoline, quinolizine, quinoxaline, quinazoline, tetrahydroisoquinoline, tetrahydroquinoline, thiopyranopyridine, and the like. These rings may be optionally substituted with groups selected from halo, alkyl, haloalkyl, alkenyl, alkynyl, cycloalkyl, cycloalkylalkyl, aryl, arylalkyl, heterocyclo, heterocycloalkyl, hydroxyl, alkoxy, alkenyloxy, alkynyloxy, haloalkoxy, cycloalkoxy, cycloalkylalkyloxy, aryloxy, arylalkyloxy, heterocyclooxy, heterocyclolalkyloxy, mercapto, amino, alkylamino, alkenylamino, alkynylamino, haloalkylamino, cycloalkylamino, cycloalkylalkylamino, arylamino, arylalkylamino, heterocycloamino, heterocycloalkylamino, disubstituted-amino, ester, amide, sulfonamide, nitro or cyano. A 5-6 membered heterocycloalkyl also includes a cyclic acetal formed from the reaction of a ketone or aldehyde with a diol such as ethylene glycol (e.g., as shown in Formula A-3ii).

“Aryl” as used herein alone or as part of another group, refers to a monocyclic carbocyclic ring system or a bicyclic carbocyclic fused ring system having one or more aromatic rings. Representative examples of aryl include, azulenyl, indanyl, indenyl, naphthyl, phenyl, tetrahydronaphthyl, and the like. The term “aryl” is intended to include both substituted and unsubstituted aryl unless otherwise indicated and these groups may be substituted with the same groups as set forth in connection with alkyl above.

“Arylalkyl” as used herein alone or as part of another group, refers to an aryl group, as defined herein, appended to the parent molecular moiety through an alkyl group, as defined herein. Representative examples of arylalkyl include, but are not limited to, benzyl, 2-phenylethyl, 3-phenylpropyl, 2-naphth-2-ylethyl, and the like.

“Heteroaryl” as used herein is as described in connection with heterocycloalkyl above.

“Alkoxy” as used herein alone or as part of another group, refers to an alkyl group, as defined herein (and thus including substituted versions such as polyalkoxy), appended to the parent molecular moiety through an oxy group, —O—. Representative examples of alkoxy include, but are not limited to, methoxy, ethoxy, propoxy, 2-propoxy, butoxy, tert-butoxy, pentyloxy, hexyloxy and the like.

“Halo” or “halogen” as used herein refers to any suitable halogen, including F, Cl, Br, and I.

“Cyano” as used herein refers to a —CN group.

“Hydroxyl” as used herein refers to an —OH group.

“Amino” as used herein means the radical —NH₂.

“Alkylamino” as used herein alone or as part of another group means the radical —NHR, where R is an alkyl group.

“Disubstituted-amino” as used herein alone or as part of another group means the radical —NR^{a}R^{b}, where R^{a} and R^{b} are independently selected from the group alkyl, haloalkyl, alkenyl, alkynyl, cycloalkyl, cycloalkylalkyl, aryl, arylalkyl, heterocyclo, heterocycloalkyl.

“Ester” as used herein alone or as part of another group refers to a —C(O)OR radical, where R is any suitable substituent such as alkyl, cycloalkyl, alkenyl, alkynyl or aryl.

“Amide” as used herein alone or as part of another group refers to a —C(O)NR^{a}R^{b} radical, where R^{a} and R^{b} are any suitable substituent such as alkyl, cycloalkyl, alkenyl, alkynyl or aryl.

As used herein, the notation “+/+” refers to genetically unmodified or wildtype alleles. As used herein, the notation “−/−” refers to genetically silenced, deleted or “knocked out” alleles. The notation “−/−” refers to a complete knock-out of that specific allele.

As used herein, the term “residue” or “residue of” a chemical moiety refers to a chemical moiety that is bound to a molecule, whereby through the binding, at least one covalent bond has replaced at least one atom of the original chemical moiety, resulting in a residue of the chemical moiety in the molecule.

The compounds of the present disclosure may have asymmetric centers. Compounds of the present disclosure containing an asymmetrically substituted atom may be isolated in optically active or racemic forms. It is well known in the art how to prepare optically active forms, such as by resolution of materials. All chiral, diastereomeric, all mixtures of chiral or diastereomeric forms and racemic forms are within the scope of this disclosure, unless the specific stereochemistry or isomeric form is specifically indicated. It will also be well recognized by a person skilled in the art that when a bond is drawn from an optically active center, that a “flat” bond ( [symbol] ) represents and encompasses both the “wedge” bond ( [symbol] ) and the “dashed” bond ( [symbol] ) each representing the (R) or(S) stereoisomer. It will also be understood by a person of ordinary skill in the art that when a compound is denoted as (R) stereoisomer, it may contain the corresponding(S) stereoisomer as an impurity i.e., the(S) stereoisomer in less than about 5%, preferably 2% by wt. and then it is denoted as a mixture of R and S isomers, the amounts of R or S isomer in the mixture is greater than about 5%, preferably 2% w/w.

The compounds of Formula A and Formula I as described herein can exist as cis and trans isomers, for example, when the compound contains a cyclohexyl ring, such as

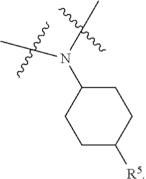

This flat structure depicts all ring conformations. Specific cis or trans conformations are depicted with bold and dashed bonds:

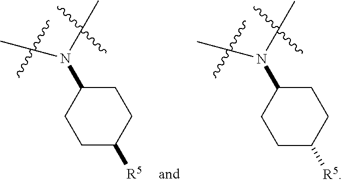

As used herein, the term “physiological conditions” refers to the range of conditions of temperature, pH, and tonicity (or osmolality) normally encountered within tissues in the body of a living human.

The term “in vitro” refers to artificial environments and to processes or reactions that occur within an artificial environment (e.g., a test tube).

The term “in vivo” refers to natural environments (e.g., a cell or organism or body) and to processes or reactions that occur within a natural environment.

“Optional” or “optionally” means that the subsequently described event or circumstance may but need not occur, and that the description includes instances where the event or circumstance occurs and instances in which it does not. For example, “heterocyclyl group optionally substituted with an alkyl group” means that the alkyl may but need not be present, and the description includes situations where the heterocyclyl group is substituted with an alkyl group and situations where the heterocyclyl group is not substituted with alkyl.

A “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, primates, such as monkeys, chimpanzees and apes, and, in particular, humans. In some embodiments, the subject can be human. In some embodiments, the subject can be a human child and/or a human infant, for example, a child or infant with a fever. In other embodiments, the subject can be a human adult.

“Treating” or “treatment” of a disease includes:
(1) preventing the disease, i.e. causing the clinical symptoms of the disease not to develop in a subject that may be exposed to or predisposed to the disease but does not yet experience or display symptoms of the disease;
(2) inhibiting the disease, i.e., arresting or reducing the development of the disease or its clinical symptoms; or
(3) relieving the disease, i.e., causing regression of the disease or its clinical symptoms.

A “therapeutically effective amount” means the amount of a compound of the present disclosure or a pharmaceutically acceptable salt thereof that, elicits the biological or medicinal response indicated. For example, when administered to a subject for treating a disease, the therapeutically effective amount of a compound is sufficient to prevent, alleviate or ameliorate symptoms of disease or prolong the survival of the subject being treated. This response may occur in a tissue, system, animal or human and includes alleviation of the signs or symptoms of the disease being treated. Determination of an effective amount is well within the capability of those skilled in the art, in view of the disclosure provided herein. The “therapeutically effective amount” of the compounds disclosed herein will vary depending on the compound, the disease and its severity and the age, weight, etc., of the subject to be treated. Designation of a range of values includes all integers within or defining the range, and all subranges defined by integers within the range.

Unless otherwise apparent from the context, the term “about” encompasses values within a standard margin of error of measurement (e.g., SEM) of a stated value or variations ±0.5%, 1%, 5%, or 10% from a specified value.

Compositions or methods “comprising” or “including” one or more recited elements may include other elements not specifically recited. For example, a composition that “comprises” or “includes” a protein may contain the protein alone or in combination with other ingredients.

The singular forms of the articles “a,” “an,” and “the” include plural references unless the context clearly dictates otherwise. For example, the term “an antigen” or “at least one antigen” can include a plurality of antigens, including mixtures thereof. Statistically significant means p≤0.05.

### III. Compounds

In certain embodiments, the subject matter described herein is directed to compounds of Formula A:

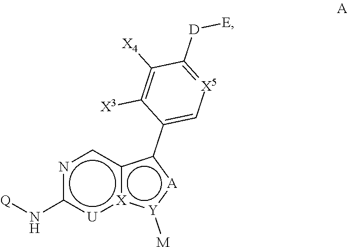

wherein:
U is N or CH; X is N or C; Y is N or C, and A is N or CH, wherein
   two or three of U, X, Y and A is N, and at least one of U and X is N;
D is selected from the group consisting of a bond, (CH₂)ₘ, (CD₂)ₘ, (CHD)ₘ, (CF₂)ₘ, (CHF)ₘ, —O—, —CO—, —N(H)— and —S(O₂)—;
E is selected from the group consisting of:
   —NR^{E1}R^{E2}, wherein R^{E1} is hydrogen, deuterium or C₁-C₆ alkyl, and R^{E2} is substituted or unsubstituted C₁-C₆ alkyl; and,

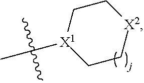

wherein:
X¹ is CH or N;
X² is CH₂, O, S, or N—R⁶, wherein R⁶ is hydrogen, substituted or unsubstituted C₁-C₆ alkyl, or C₃-C₉ cycloalkyl;
X³ and X⁴ are independently selected from hydrogen and halogen;
X⁵ is CH or N;
Q is selected from the group consisting of:

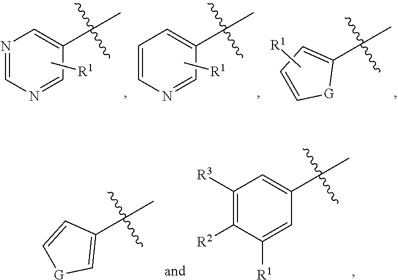

wherein, G is S, O or N;
M is

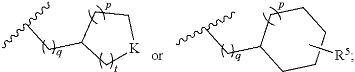

K, if present, is O or CR⁷R⁸, wherein R⁷ and R⁸ are independently selected from C₁-C₆ alkylhydroxy, or R⁷ and R⁸ together with the carbon to which each is attached form a carbonyl or a 5-6 membered heterocycloalkyl;
m is 0, 1, or 2;
j is 0, 1, 2, or 3;
p is 0 or 1;
t is 1 or 2;
q is 0 or 1;
R¹ is selected from the group consisting of halogen, C₁-C₆ alkyl, C₁-C₆ alkoxy, hydroxy, nitro, C₁-C₆-haloalkyl, cyano, and N(R^{4a})₂,
   wherein each R^{4a} is independently selected from the group consisting of hydrogen, and C₁-C₆-alkyl;
R² and R³ are each independently selected from the group consisting of hydrogen, deuterium, halogen, C₁-C₆ alkyl, C₁-C₆ alkoxy, hydroxy, nitro, C₁-C₆-haloalkyl, cyano, and N(R^{4b})₂,
   wherein each R^{4b} is independently selected from the group consisting of hydrogen and C₁-C₆-alkyl;
      or,
R¹ and R² or R² and R³ together with the carbon to which each is attached form a ring selected from the group consisting of substituted or unsubstituted C₃-C₈ cycloalkyl, substituted or unsubstituted C₃-C₈ heterocycloalkyl, substituted or unsubstituted C₃-C₈ aryl, and substituted or unsubstituted C₃-C₈ heteroaryl, wherein the heteroatom is O, N, or S; and,
R⁵ is selected from the group consisting of hydroxy, cyano, halogen, amino, C₁-C₆ alkyl, C₁-C₆ alkoxy, mono(C₁-C₆)alkylamino di(C₁-C₆)alkylamino, N-alkoxycarbonyl, and C₁-C₆ alkylhydroxy;
   or a pharmaceutically acceptable salt thereof; wherein the circle inside a ring depicts single or double bonds, aromatic and non-aromatic, as appropriate for the value of U, X, Y and A.

In certain embodiments, the subject matter described herein is directed to compounds of Formula A-1:

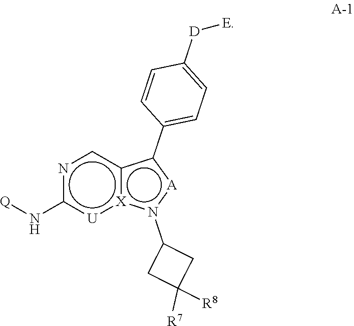

In certain embodiments, the subject matter described herein is directed to compounds of Formula A-2:

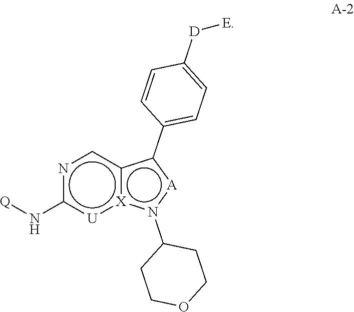

In certain embodiments, the subject matter described herein is directed to compounds of Formula A-3:

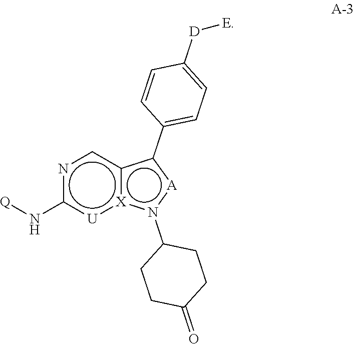

In certain embodiments, the subject matter described herein is directed to compounds of Formula A-4:

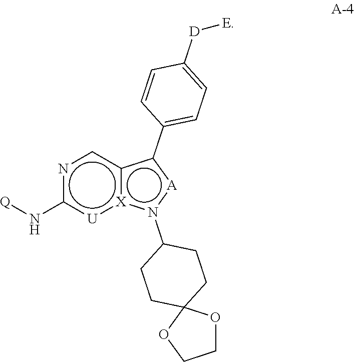

In certain embodiments, the subject matter described herein is directed to compounds of Formula A-5:

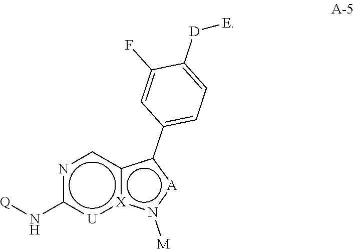

In certain embodiments, the subject matter described herein is directed to compounds of Formula A-6:

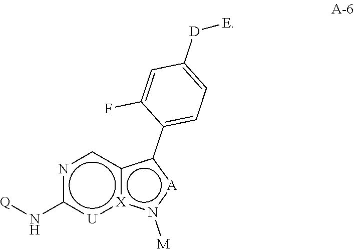

In certain embodiments, the subject matter described herein is directed to compounds of Formula A-7:

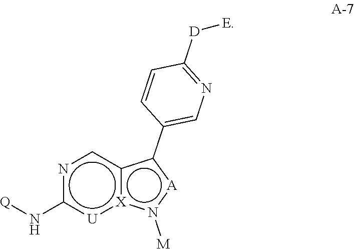

In certain embodiments, the subject matter described herein is directed to compounds of Formula I:

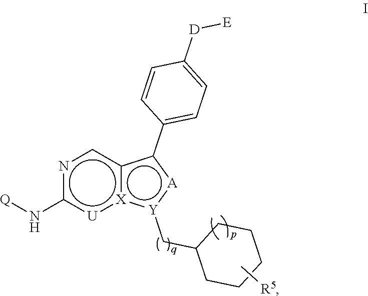

wherein:
U is N or CH; X is N or C; Y is N or C, and A is N or CH, wherein
   two or three of U, X, Y and A is N, and at least one of U and X is N; D is selected from the group consisting of a bond, (CH₂)ₘ, (CD₂)ₘ, (CHD)ₘ, (CF₂)ₘ, (CHF)ₘ, —O—, —CO—, —N(H)— and —S(O₂)—;
E is selected from the group consisting of:
   —NR^{E1}R^{E2}, wherein R^{E1} is hydrogen or C₁-C₆ alkyl, and R^{E2} is substituted or unsubstituted C₁-C₆ alkyl; and,

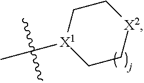

wherein:
X¹ is CH or N;
X² is CH₂, O, S, or N—R⁶, wherein R⁶ is hydrogen, substituted or unsubstituted C₁-C₆ alkyl, or C₃-C₉ cycloalkyl; Q is selected from the group consisting of:

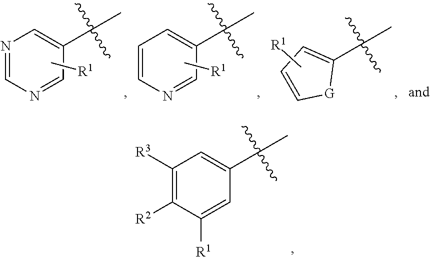

wherein, G is S, O or N;
m is 0, 1, or 2;
j is 0, 1, 2, or 3;
p is 0 or 1;
q is 0 or 1;
R¹ is selected from the group consisting of halogen, C₁-C₆ alkyl, C₁-C₆ alkoxy, hydroxy, nitro, C₁-C₆-haloalkyl, cyano, and N(R^{4a})₂,
   wherein each R^{4a} is independently selected from the group consisting of hydrogen, and C₁-C₆-alkyl;
R² and R³ are each independently selected from the group consisting of hydrogen, halogen, C₁-C₆ alkyl, C₁-C₆ alkoxy, hydroxy, nitro, C₁-C₆-haloalkyl, cyano, and N(R^{4b})₂,
   wherein each R^{4b} is independently selected from the group consisting of hydrogen and C₁-C₆-alkyl;
      or,
R¹ and R² or R² and R³ together with the carbon to which each is attached form a ring selected from the group consisting of substituted or unsubstituted C₃-C₈ cycloalkyl, substituted or unsubstituted C₃-C₈ heterocycloalkyl, substituted or unsubstituted C₃-C₈ aryl, and substituted or unsubstituted C₃-C₈ heteroaryl, wherein the heteroatom is O, N, or S; and,
R⁵ is selected from the group consisting of hydroxy, cyano, halogen, amino, C₁-C₆ alkyl, C₁-C₆ alkoxy, mono(C₁-C₆)alkylamino and di(C₁-C₆)alkylamino; or R⁵ is selected from the group consisting of hydroxy, cyano, halogen, amino, C₁-C₆ alkyl, C₁-C₆ alkoxy, mono(C₁-C₆)alkylamino and di(C₁-C₆)alkylamino, N-alkoxycarbonyl, C₁-C₆ alkylhydroxy; or a pharmaceutically acceptable salt thereof; wherein the circle inside a ring depicts single or double bonds, aromatic and non-aromatic, as appropriate for the value of U, X, Y and A.

In certain embodiments, the subject matter described herein is directed to compounds of Formula I, wherein:
U is N; X is C; Y is N; and A is CH, the compound having a structure of Formula I-1:

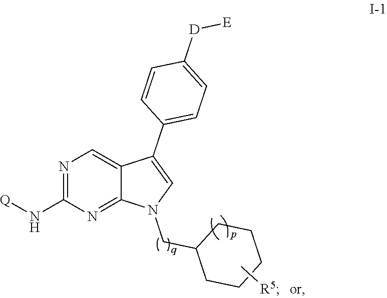

U is N; X is C; Y is N; and A is N, the compound having a structure of Formula I-2:

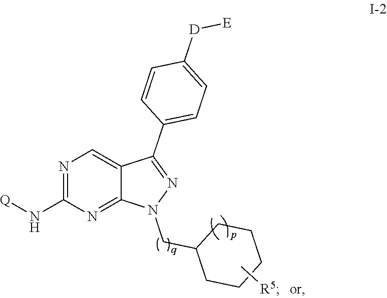

U is N; X is N; Y is C; and A is N, the compound having a structure of Formula I-3:

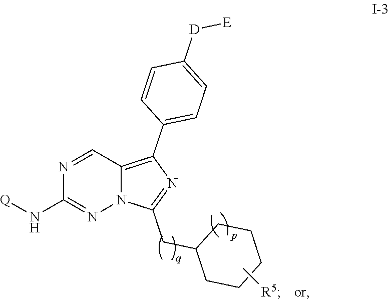

U is N; X is N; Y is C; and A is CH, the compound having a structure of Formula I-4:

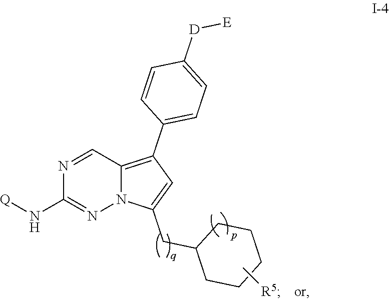

U is CH; X is N; Y is C; and A is N, the compound having a structure of Formula I-5:

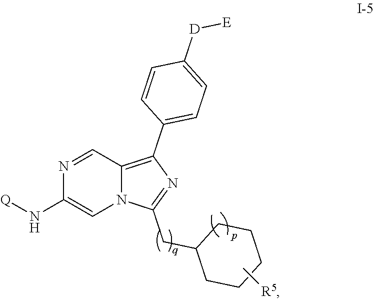

or a pharmaceutically acceptable salt thereof.

In certain embodiments, the compounds are of Formula I-1 or I-2, having a structure of Formula Ia:

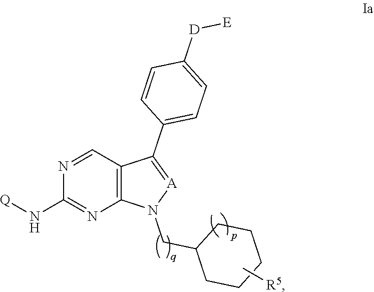

or a pharmaceutically acceptable salt thereof.

In certain embodiments, the compounds are of Formula I-3, I-4 or I-5, having a structure of Formula Ib:

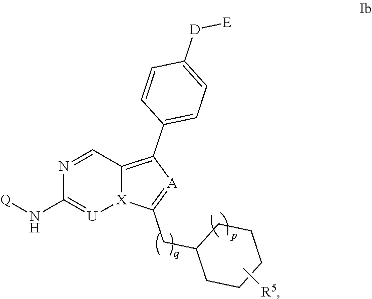

or a pharmaceutically acceptable salt thereof.

In the above embodiments, compounds include those wherein E is

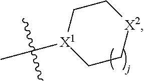

In the above embodiments, compounds include those wherein Q is selected from the group consisting of:

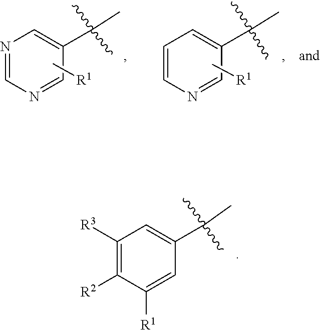

In the above embodiments, compounds include those wherein Q is

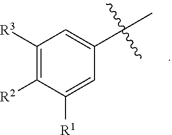

In the above embodiments, compounds include those wherein
R¹ is selected from the group consisting of halogen, C₁-C₆ alkyl, C₁-C₆ alkoxy, hydroxy, nitro, C₁-C₆-haloalkyl, cyano, and N(R^{4a})₂, wherein each R^{4a} is independently selected from the group consisting of hydrogen, and C₁-C₆-alkyl; and,
R² and R³ are each independently selected from the group consisting of hydrogen, halogen, C₁-C₆ alkyl, C₁-C₆ alkoxy, hydroxy, nitro, C₁-C₆-haloalkyl, cyano, and N(R^{4b})₂, wherein each R^{4b} is independently selected from the group consisting of hydrogen and C₁-C₆-alkyl.

In the above embodiments, compounds include those of Formula Ia-1:

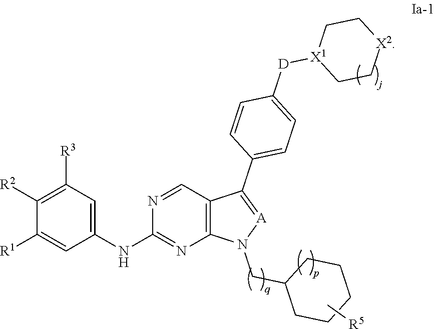

In the above embodiments, compounds include those of Formula Ib-1 or Ib-1′:

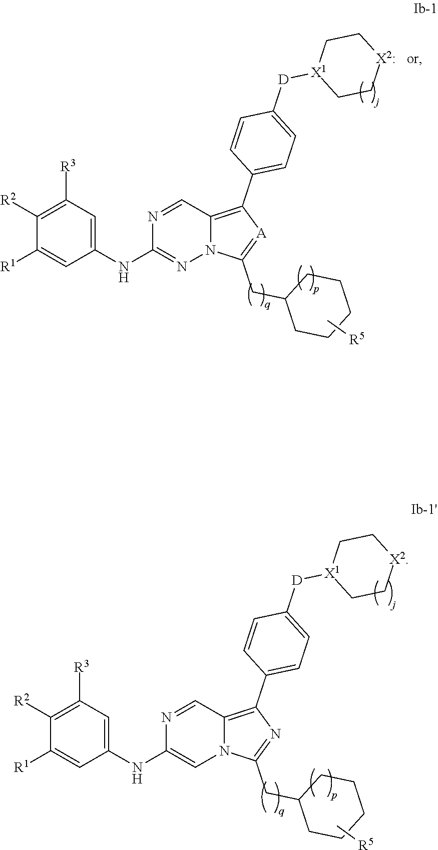

In the above embodiments, compounds include those wherein q is 0.

In the above embodiments, compounds include those wherein p is 1.

In the above embodiments, compounds include those wherein D is (CH₂)ₘ, —O— or —N(H)—.

In the above embodiments, compounds include those wherein R⁵ is selected from the group consisting of hydroxy, halogen, amino, C₁-C₆ alkyl, and C₁-C₆ alkoxy. In the above embodiments, compounds include those wherein R⁵ is selected from the group consisting of hydroxy, amino, methoxy, ethoxy, methyl and ethyl. In the above embodiments, compounds include those wherein R⁵ is hydroxy, methoxy, or ethoxy. In the above embodiments, compounds include those wherein R⁵ is hydroxy.

In the above embodiments, compounds include those of Formula Ia-2:

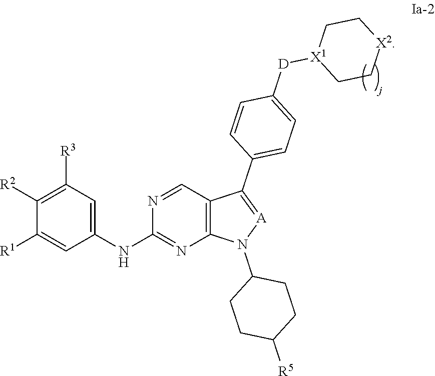

In the above embodiments, compounds include those having a structure of Formula Ib-2 or Ib-2′:

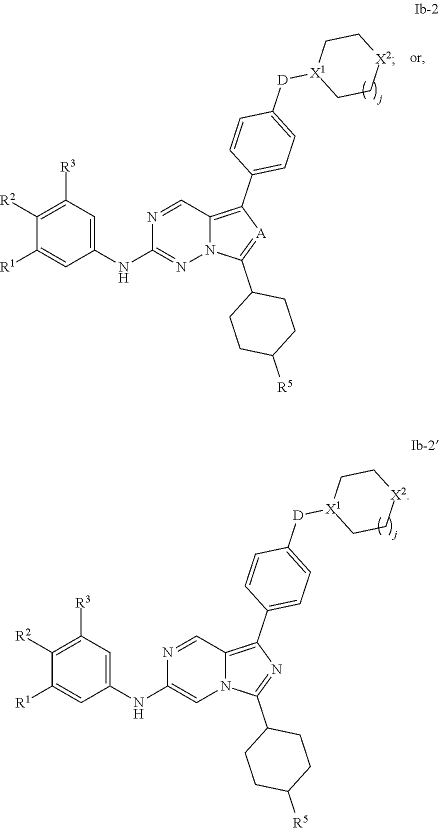

In the above embodiments, compounds include those wherein X¹ is N.

In the above embodiments, compounds include those wherein, X² is S or N—R⁶, wherein R⁶ is hydrogen or a substituted or unsubstituted C₁-C₆ alkyl, or a C₃-C₉ cycloalkyl.

In the above embodiments, compounds include those wherein X¹ is N, and X² is S or N—R⁶, wherein R⁶ is hydrogen or a substituted or unsubstituted C₁-C₆ alkyl.

In the above embodiments, compounds include those wherein j is 1 or 2.

In the above embodiments, compounds include those wherein D is —N(H)— or O.

In the above embodiments, compounds include those wherein E is

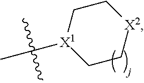

wherein X¹ is CH.

In the above embodiments, compounds include those wherein D is (CH₂)ₘ.

In the above embodiments, compounds include those wherein E is

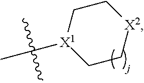

wherein X¹ is N.

In the above embodiments, compounds include those wherein A is N.

In the above embodiments, compounds include those of Formula Ia-3, Ib-3 or Ib-3′:

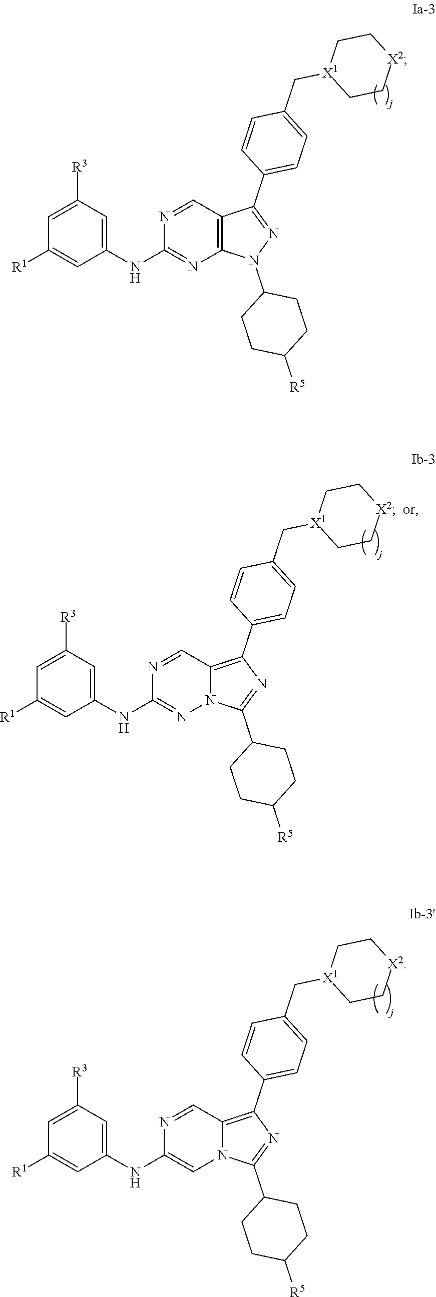

In the above embodiments, compounds include those wherein A is CH.

In the above embodiments, compounds include those of Formula Ia-4 or Ib-4:

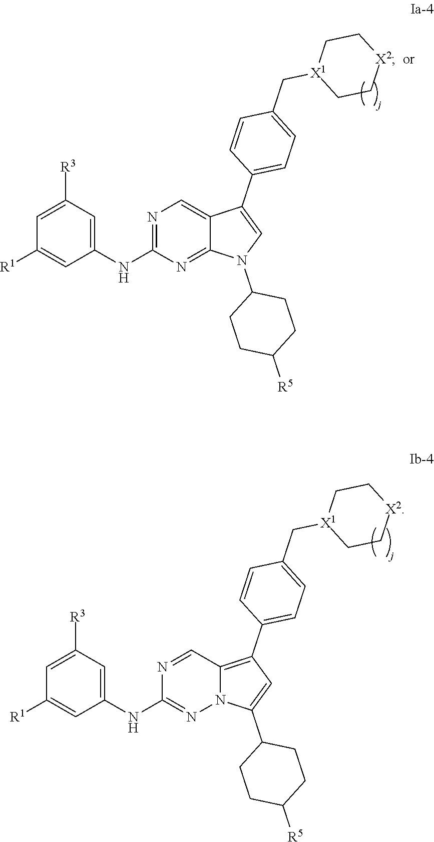

In the above embodiments, compounds include those having a structure of Formula Ia-5:

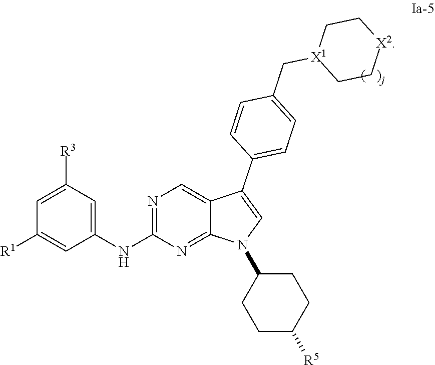

In the above embodiments, compounds include those having a structure of Formula Ia-6:

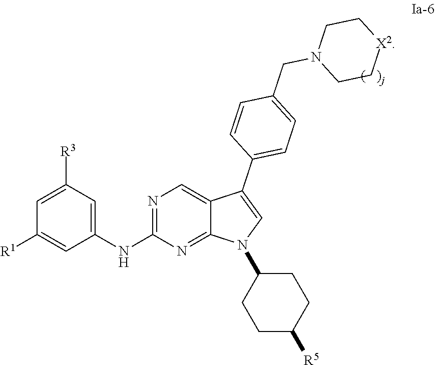

In the above embodiments, compounds include those wherein D is CH₂; and E is —NR^{E1}R^{E2}, wherein R^{E1} is hydrogen, and R^{E2} is C₁-C₆ alkyl substituted once with mono(C₁-C₆ alkyl)amino, di(C₁-C₆ alkyl)amino, or a 5-7 member heterocyclyl.

In the above embodiments, compounds include those wherein -D-E is selected from the group consisting of

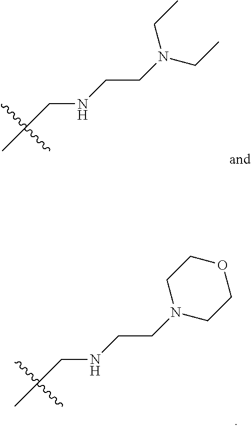

In the above embodiments, compounds include those of Formula Ic:

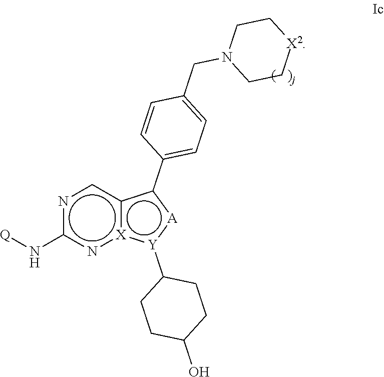

In the above embodiments, compounds include those of Formula Ic-1:

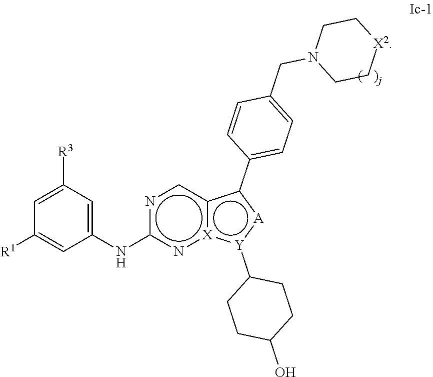

In the above embodiments, compounds include those of Formula Ic-2:

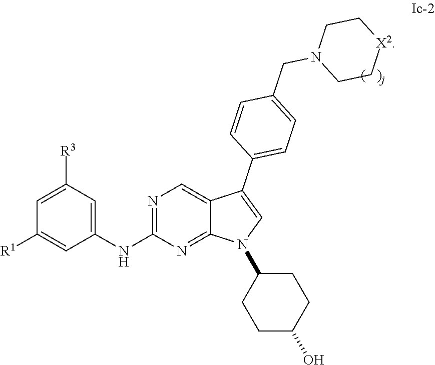

In the above embodiments, compounds include those of Formula Ic-3:

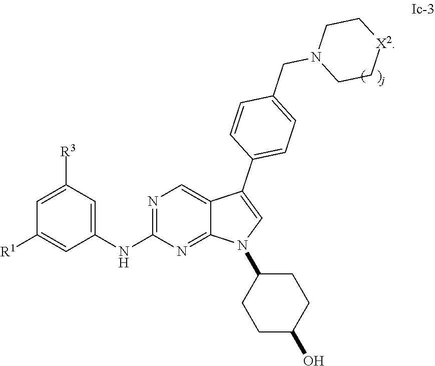

In the above embodiments, compounds include those wherein X² is S.

In the above embodiments, compounds include those wherein X² is N—R⁶, wherein R⁶ is C₁-C₆ alkyl.

In the above embodiments, compounds include those wherein R⁶ is methyl.

In the above embodiments, compounds include those wherein
R¹ is selected from the group consisting of halogen, C₁-C₆ haloalkyl and C₁-C₆ alkoxy; and,
R³ is selected from the group consisting of halogen, C₁-C₆ haloalkyl and C₁-C₆ alkoxy.

In the above embodiments, compounds include those wherein
R¹ is selected from the group consisting of fluoro, —CF₃ and methoxy; and,
R³ is selected from the group consisting of chloro, —CF₃ and methoxy.

In the above embodiments, compounds include those where X³ is selected from the group consisting of H, F, Cl, and Br. In certain embodiments, X³ is F.

In the above embodiments, compounds include those where X⁴ is selected from the group consisting of H, F, Cl, and Br. In certain embodiments, X⁴ is F.

In the above embodiments, compounds include those where X⁵ is CH.

In the above embodiments, compounds include those where X⁵ is N.

In the above embodiments, compounds include those wherein Q is

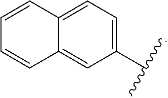 [SMILES: *c1ccc2ccccc2c1]

In the above embodiments, compounds include those wherein Q is

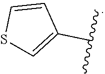 [SMILES: *c1ccsc1]

In the above embodiments, compounds include those wherein Q is

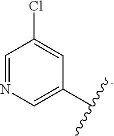 [SMILES: *c1cncc(Cl)c1]

In the above embodiments, compounds include those of Formula I, wherein R⁵ is

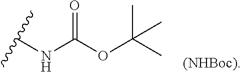 [SMILES: *NC(=O)OC(C)(C)C]

In certain embodiments, a C₁-C₆ alkylhydroxy is —CH₂OH.

In the above embodiments, compounds include those of Formula A-1, wherein R⁷ and R⁸ are —CH₂OH.

In the above embodiments, compounds include those as shown below in Table 1:

| Compound | |
|---|---|
| Number | Chemical Structure |
| 1 | 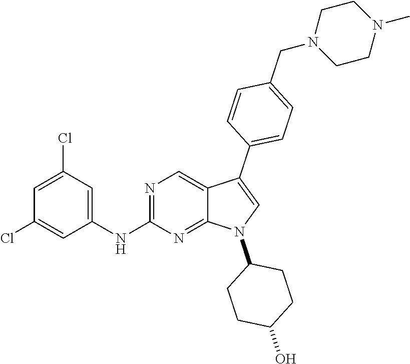 |
| 2 | 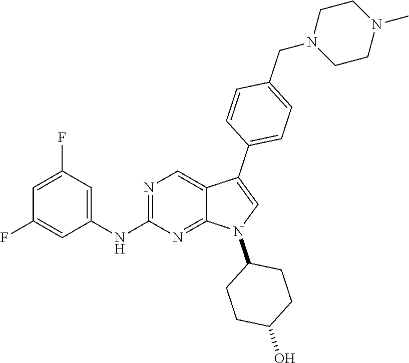 |
| 3 | 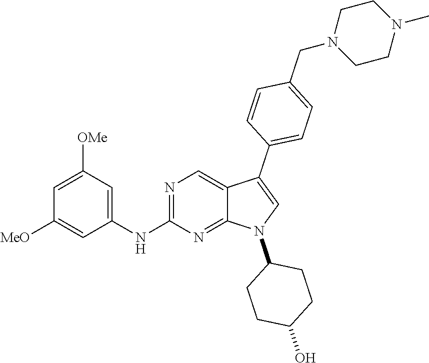 |
| 4 | 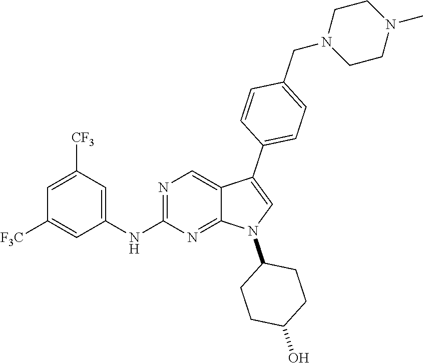 |
| 5 | 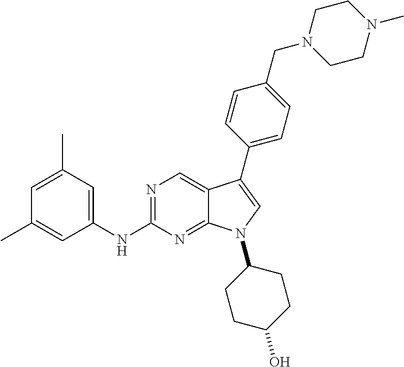 |
| 6 | 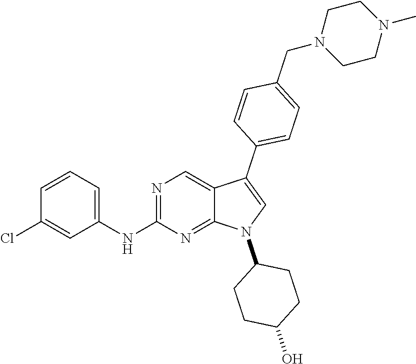 |
| 7 | 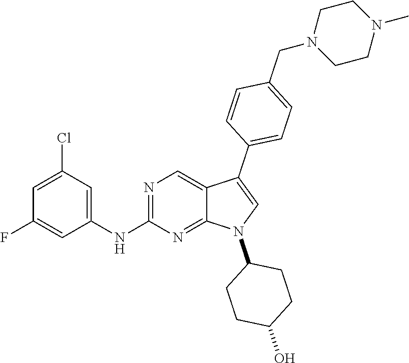 |
| 8 | 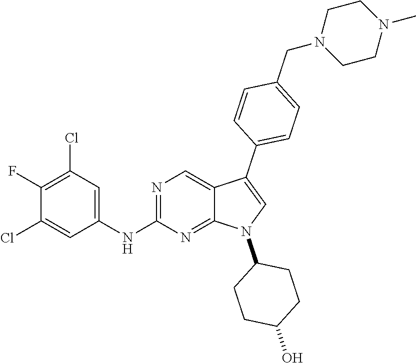 |
| 9 | 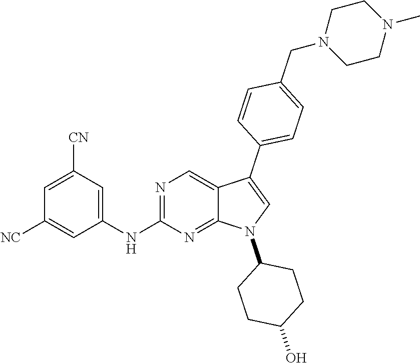 |
| 10 | 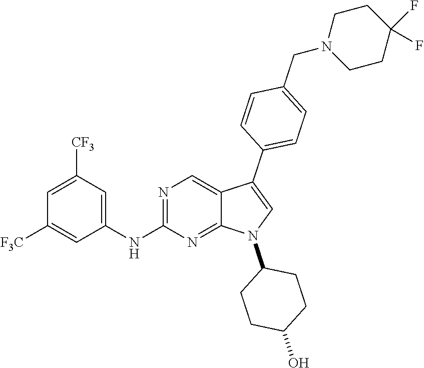 |
| 11 | 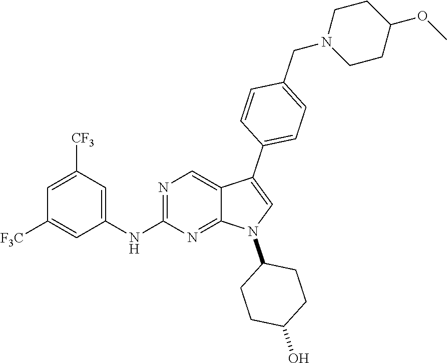 |
| 12 | 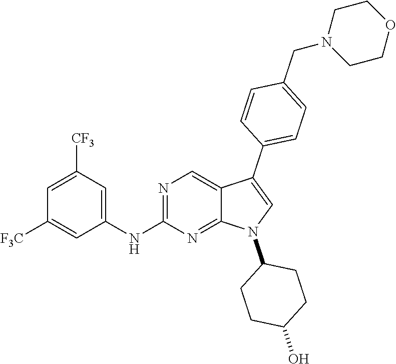 |
| 13 | 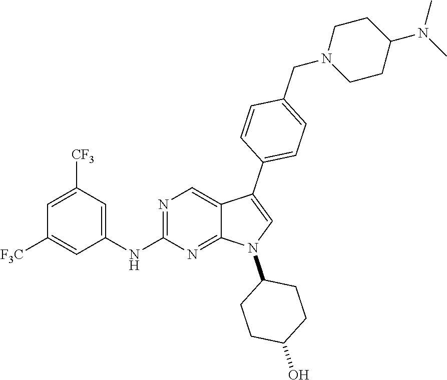 |
| 14 | 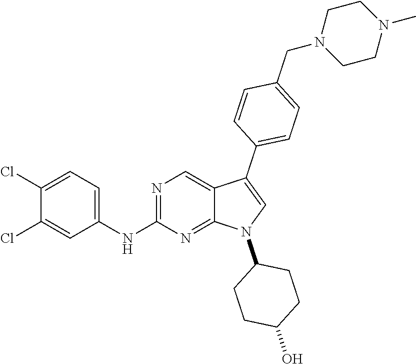 |
| 15 | 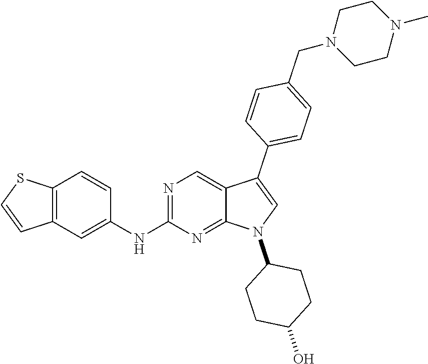 |
| 18 | 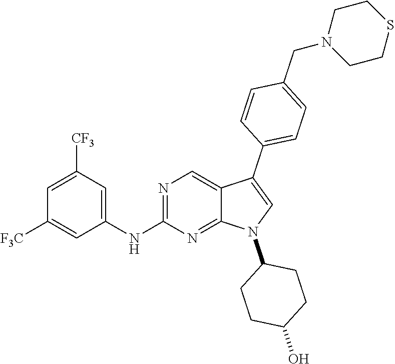 |
| 19 | 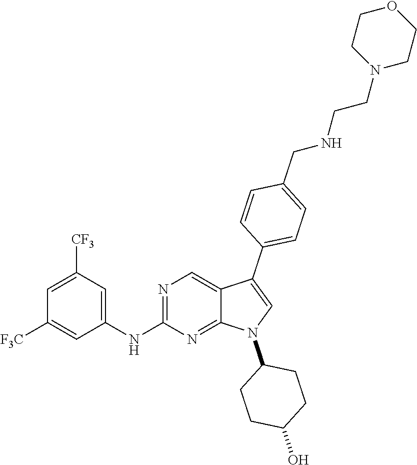 |
| 20 | 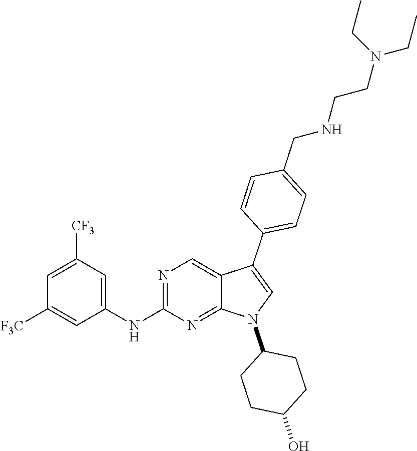 |
| 21 | 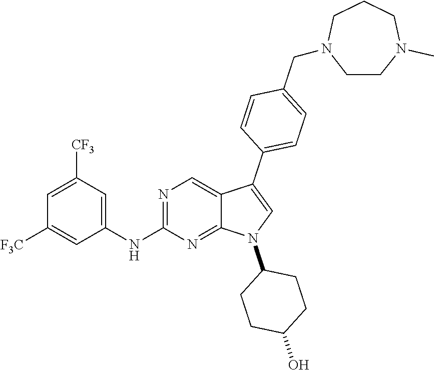 |
| 22 | 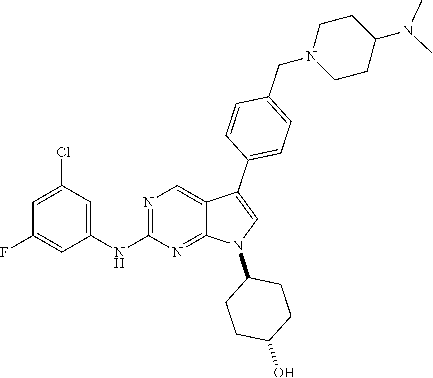 |
| 23 | 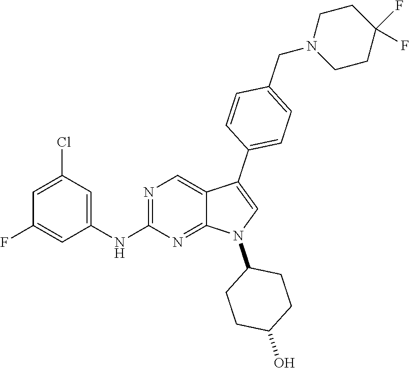 |
| 24 | 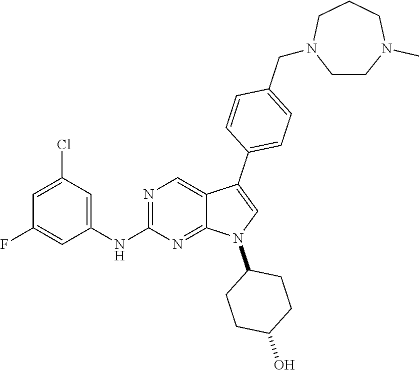 |
| 25 | 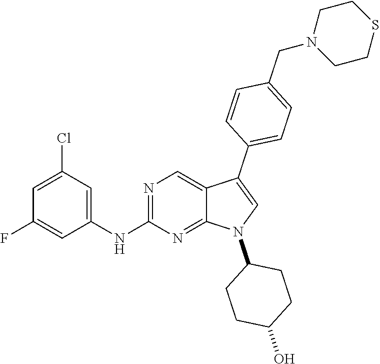 |
| 26 | 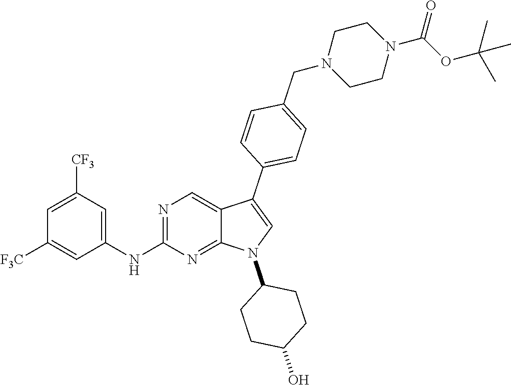 |
| 27 | 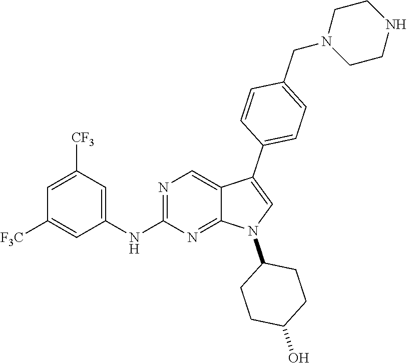 |
| 28 | 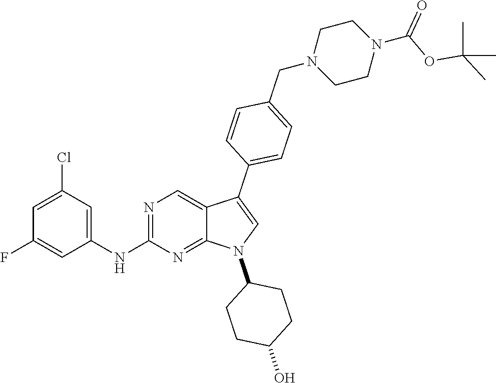 |
| 29 | 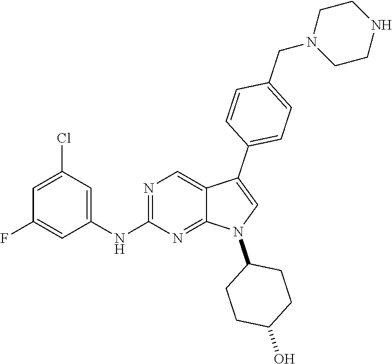 |
| 30 | 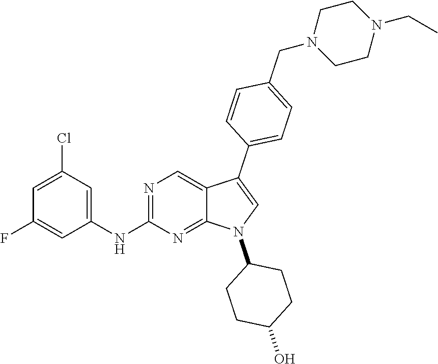 |
| 31 | 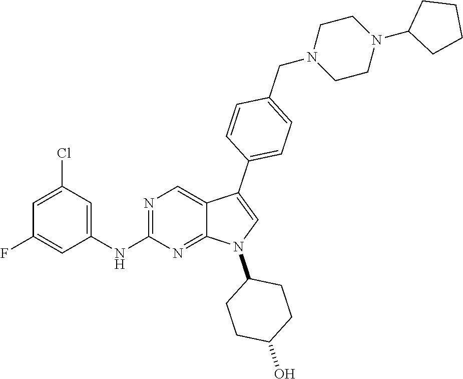 |
| 32 | 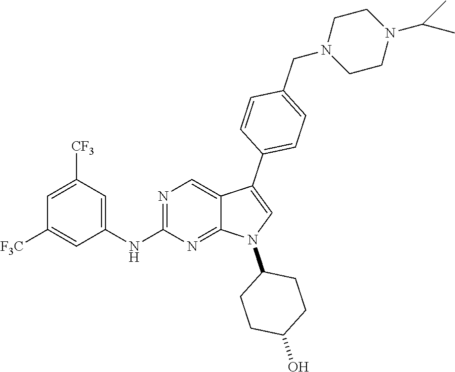 |
| 33 | 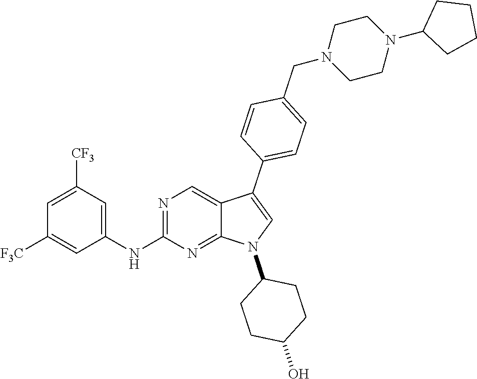 |
| 34 | 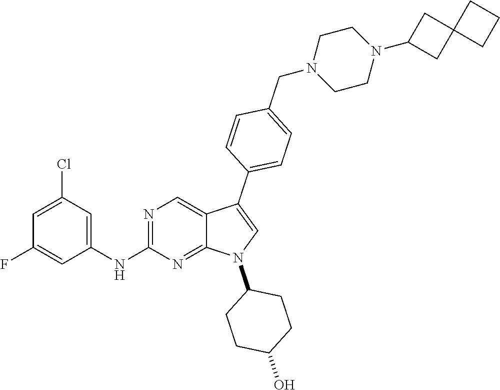 |
| 35 | 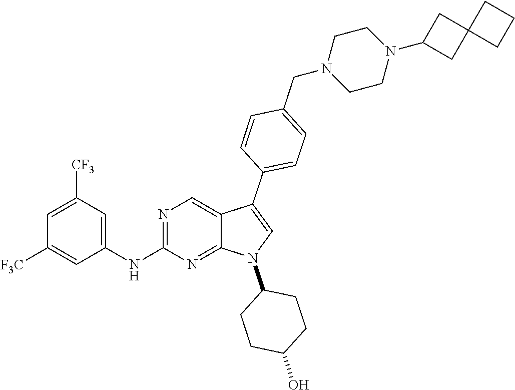 |
| 36 | 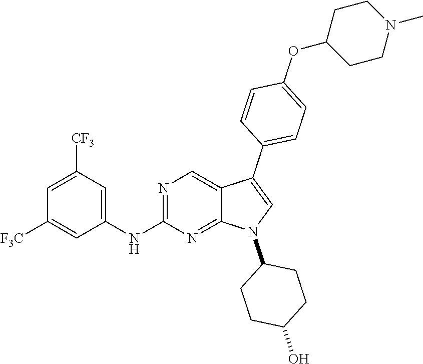 |
| 37 | 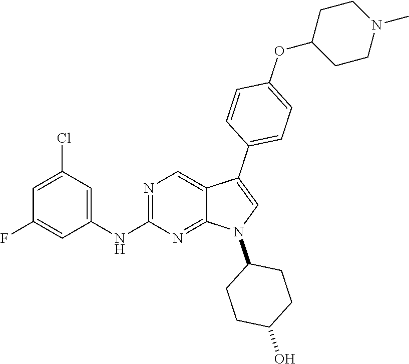 |
| 38 | 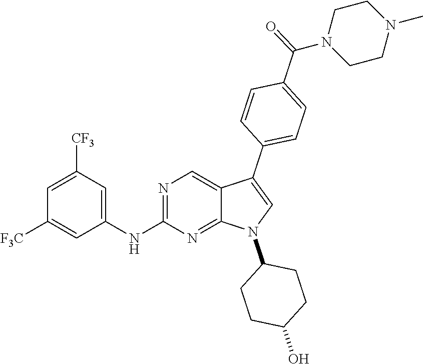 |
| 39 | 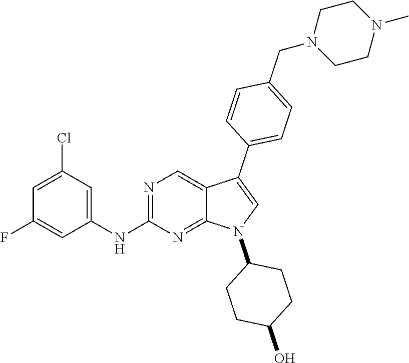 |
| 40 | 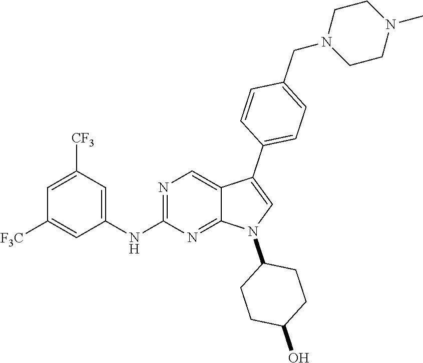 |
| 41 | 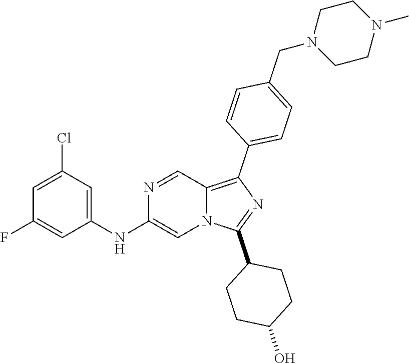 |
| 42 | 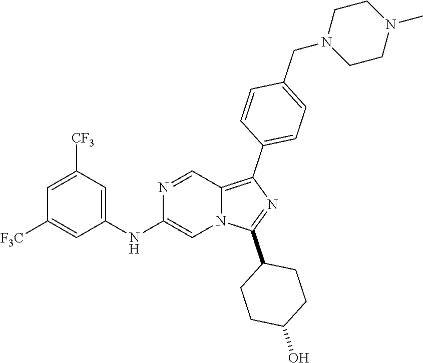 |
| 43 | 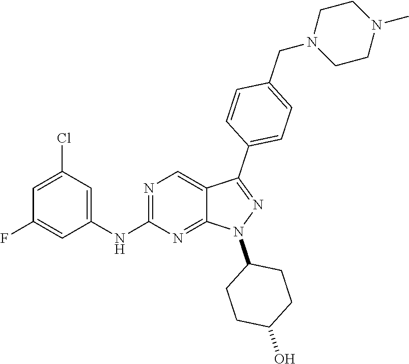 |
| 44 | 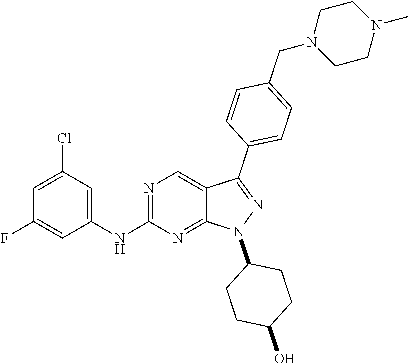 |
| 45 | 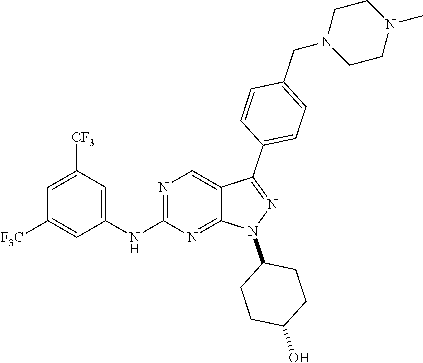 |
| 46 | 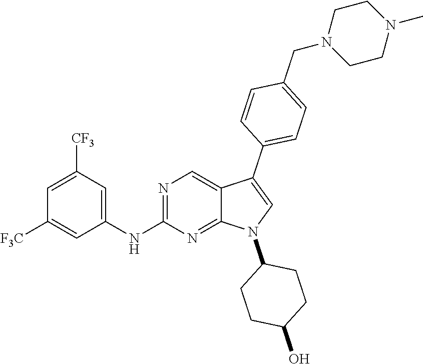 |

In the above embodiments, compounds include those as shown below in Table 1A:

| Compound | |
|---|---|
| Number | Chemical Structure |
| 47 | 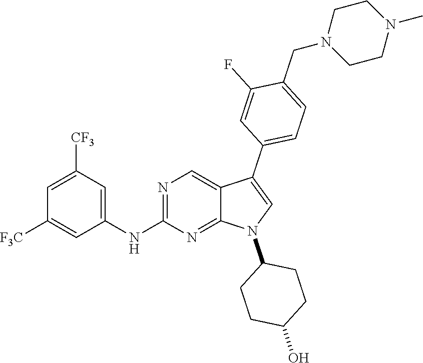 |
| 48 | 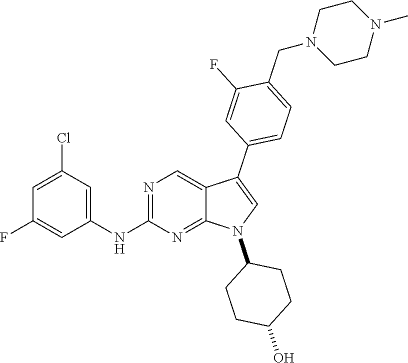 |
| 49 | 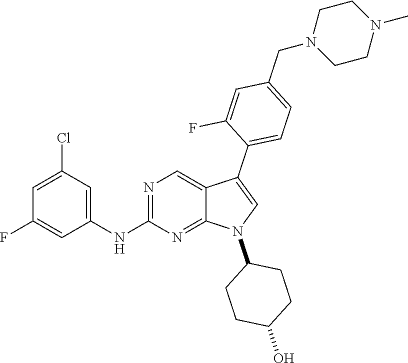 |
| 50 | 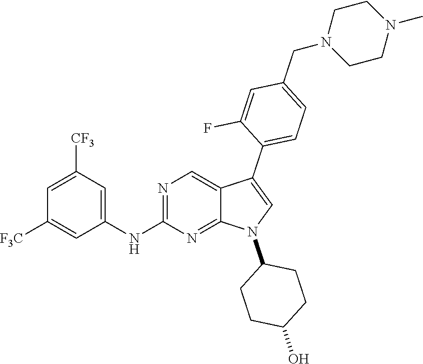 |
| 51 | 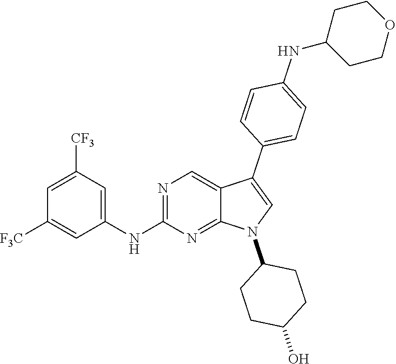 |
| 52 | 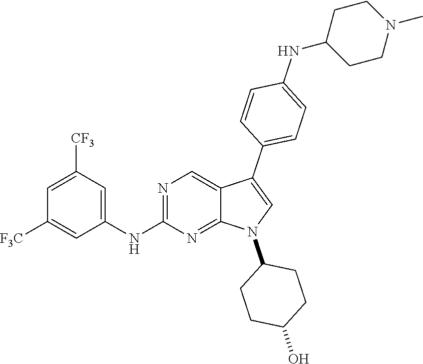 |
| 53 | 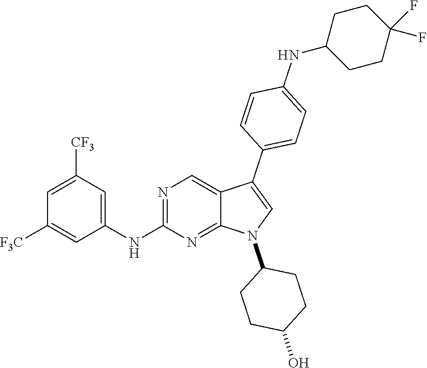 |
| 54 | 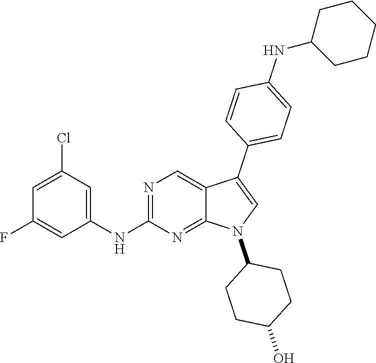 |
| 55 | 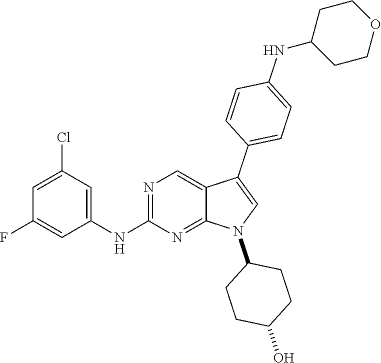 |
| 56 | 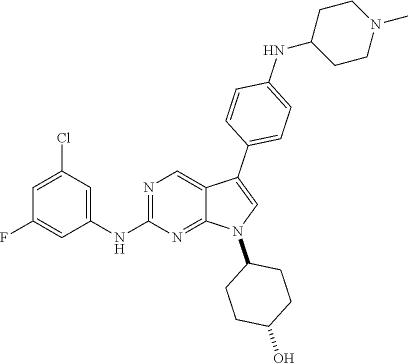 |
| 57 | 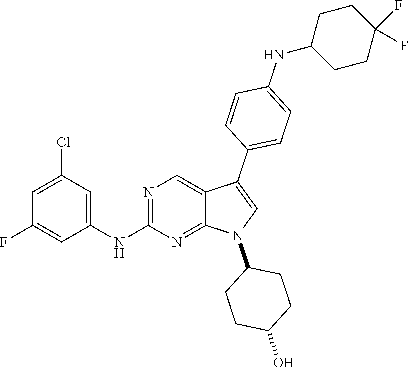 |
| 58 | 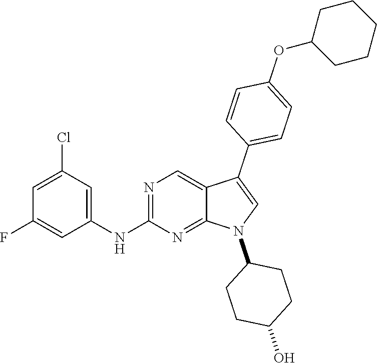 |
| 59 | 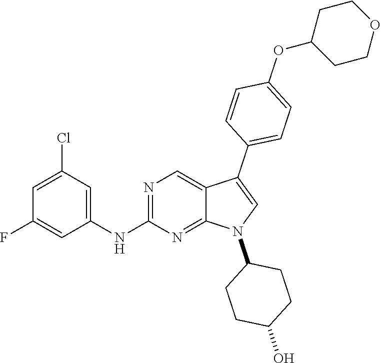 |
| 60 | 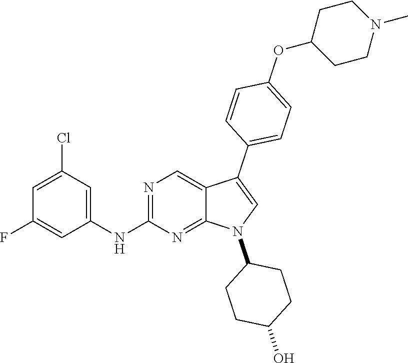 |
| 61 | 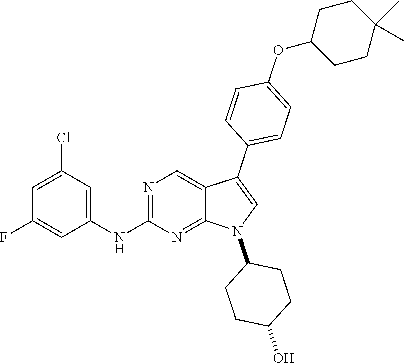 |
| 62 | 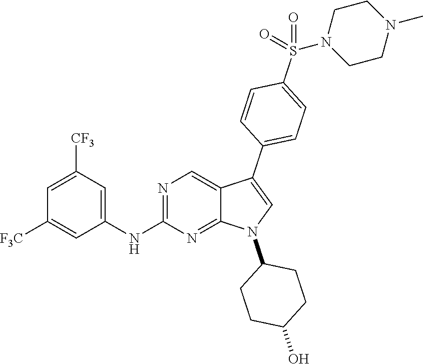 |
| 63 | 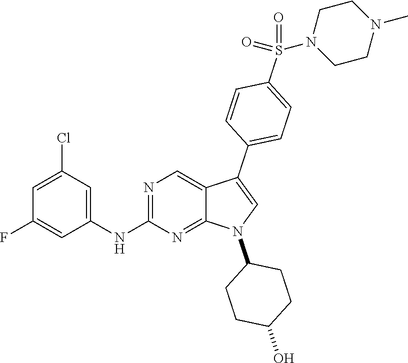 |
| 64 | 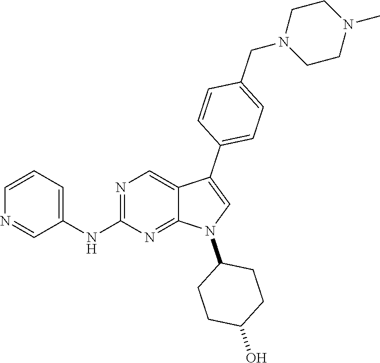 |
| 65 | 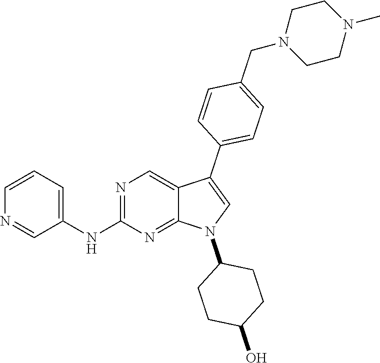 |
| 66 | 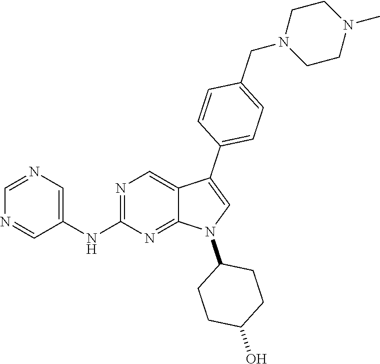 |
| 67 | 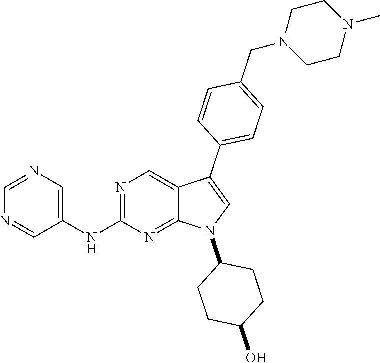 |
| 68 | 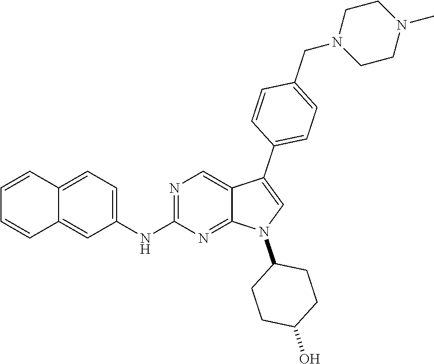 |
| 69 | 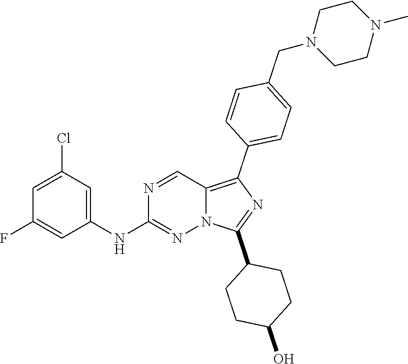 |
| 70 | 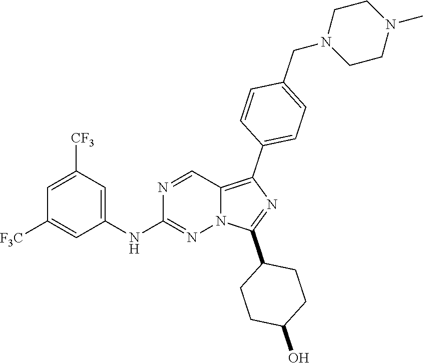 |
| 71 | 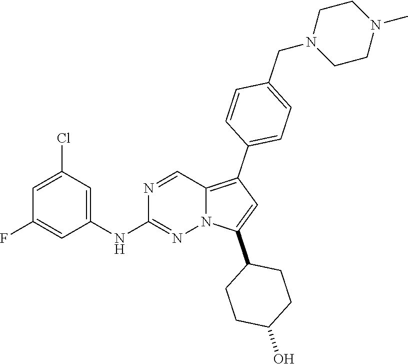 |
| 72 | 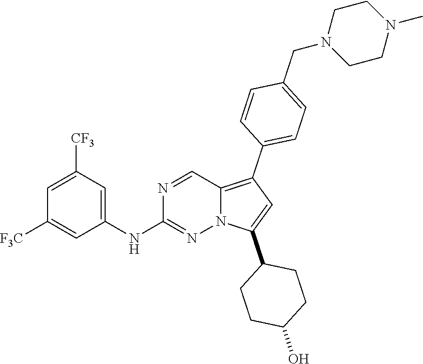 |
| 73 | 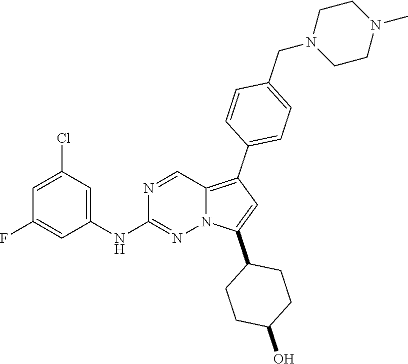 |
| 74 | 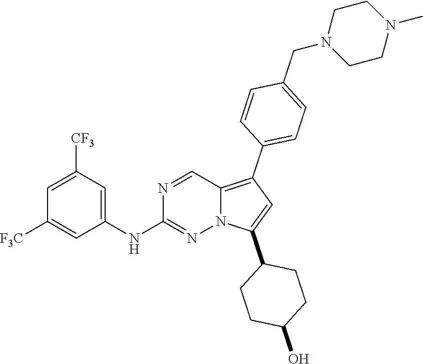 |

In the above embodiments, compounds include those as shown below in Table 1B:

| Compound | |
|---|---|
| Number | Chemical Structure |
| 141 | 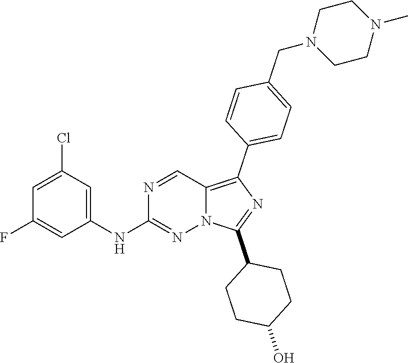 |
| 142 | 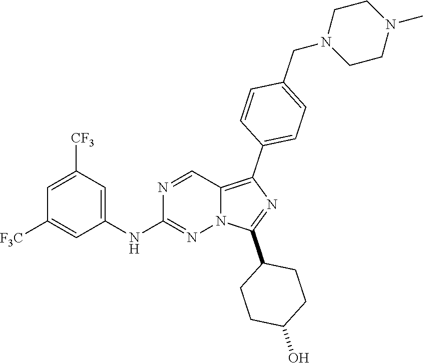 |
| 147 | 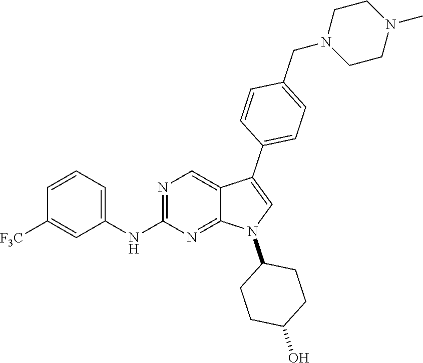 |
| 148 | 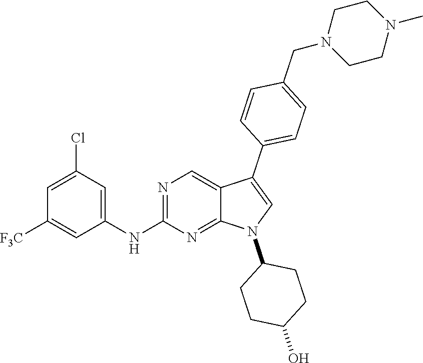 |
| 149 | 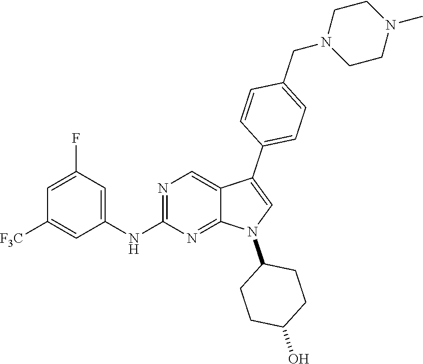 |
| 150 | 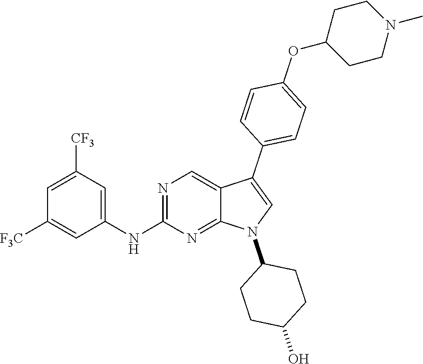 |
| 151 | 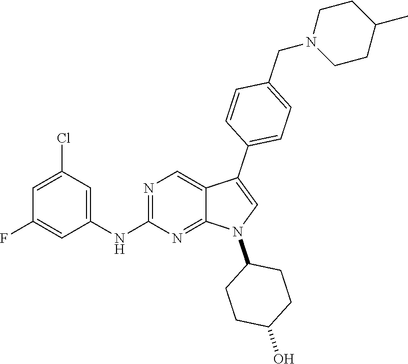 |
| 155 | 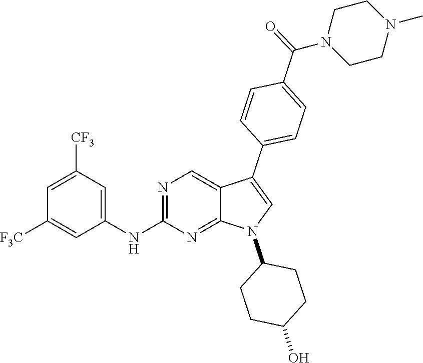 |
| 156 | 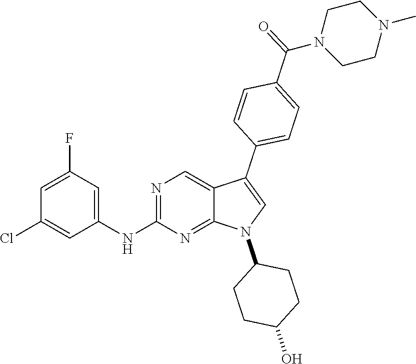 |
| 159 | 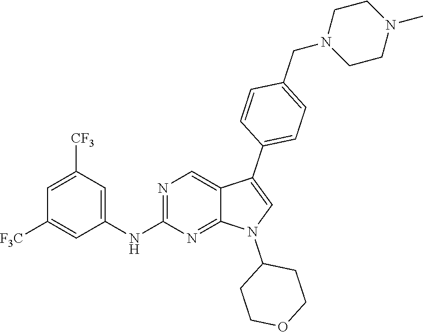 [SMILES: CN1CCN(Cc2ccc(-c3cn(C4CCOCC4)c4nc(Nc5cc(C(F)(F)F)cc(C(F)(F)F)c5)ncc34)cc2)CC1] |
| 160 | 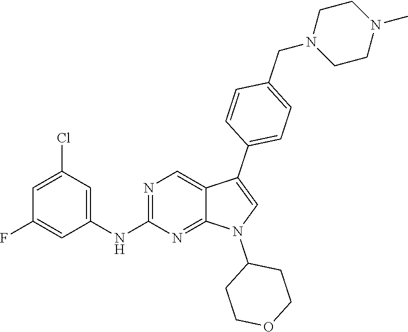 [SMILES: CN1CCN(Cc2ccc(-c3cn(C4CCOCC4)c4nc(Nc5cc(F)cc(Cl)c5)ncc34)cc2)CC1] |
| 161 | 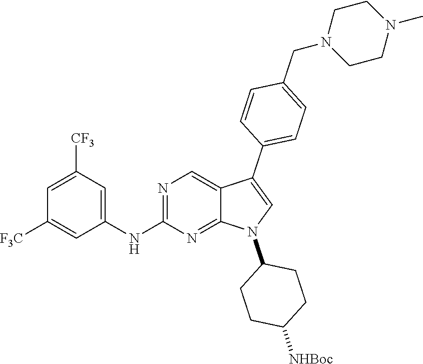 |
| 162 | 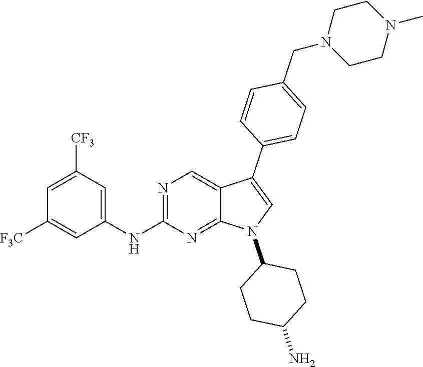 |
| 163 | 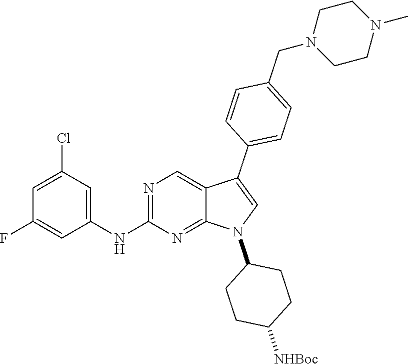 |
| 164 | 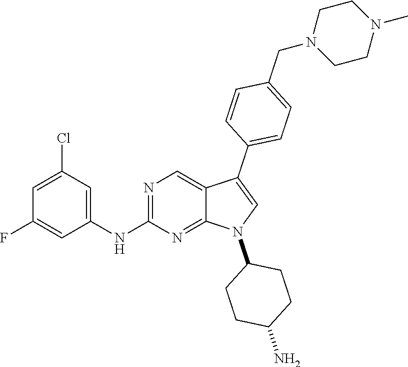 |
| 165 | 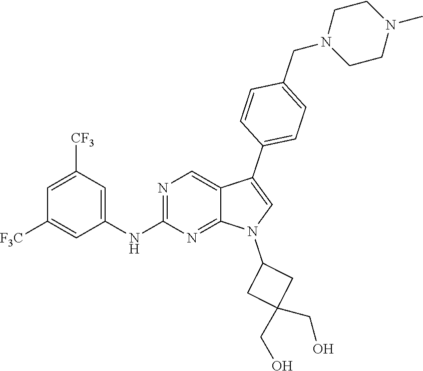 [SMILES: CN1CCN(Cc2ccc(-c3cn(C4CC(CO)(CO)C4)c4nc(Nc5cc(C(F)(F)F)cc(C(F)(F)F)c5)ncc34)cc2)CC1] |
| 170 | 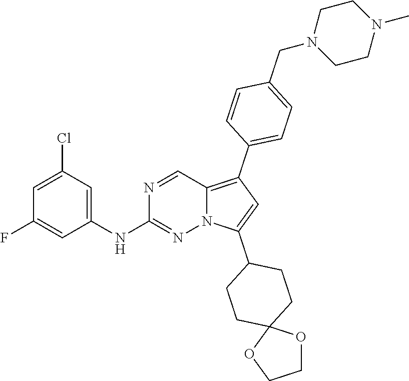 [SMILES: CN1CCN(Cc2ccc(-c3cc(C4CCC5(CC4)OCCO5)n4nc(Nc5cc(F)cc(Cl)c5)ncc34)cc2)CC1] |
| 171 | 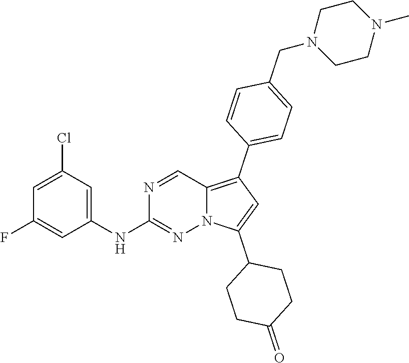 [SMILES: CN1CCN(Cc2ccc(-c3cc(C4CCC(=O)CC4)n4nc(Nc5cc(F)cc(Cl)c5)ncc34)cc2)CC1] |
| 172 | 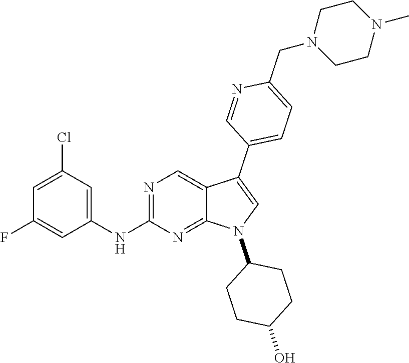 |
| 173 | 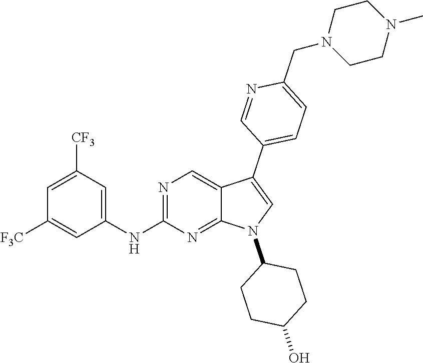 |
| 174 | 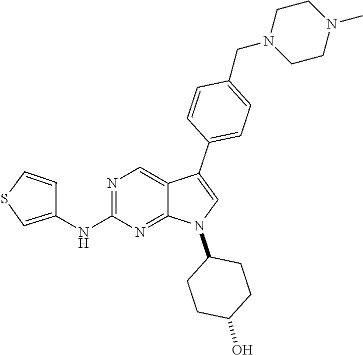 |
| 175 | 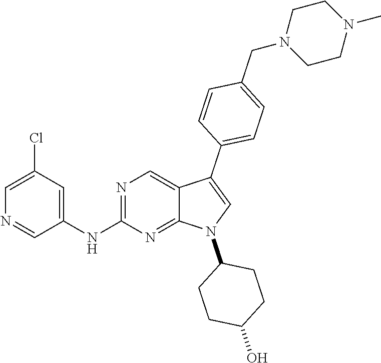 |

In certain embodiments, the compounds include those of Formula I, or pharmaceutically acceptable salts thereof, where the compounds inhibit one or more proteins of the TAM protein kinases, i.e. TYRO3, AXL, or Mer.

In some embodiments, the compounds of Formula I or pharmaceutically acceptable salts thereof, display inhibitory concentrations, IC₅₀ (s), defined as ++++, +++, ++, +, or −. An IC₅₀ of ++++ refers to an IC₅₀ of <10 nM. In embodiments described herein, ++++ refers to IC₅₀(s) of about 9 nM, about 8 nM, about 7 nM, about 6 nM, about 5 nM, about 4 nM, about 3 nM, about 2 nM, about 1 nM, or about 0.5 nM. An IC₅₀ of +++ refers to an IC₅₀ between 10 nM and 100 nM. In embodiments described herein, +++ refers to IC₅₀(s) of about 10 nM, about 11 nM, about 12 nM, about 13 nM, about 14 nM, about 15 nM, about 20 nM, about 25 nM, about 30 nM, about 35 nM, about 40 nM, about 45 nM, about 50 nM, about 55 nM, about 60 nM, about 65 nM, about 70 nM, about 75 nM, about 80 nM, about 85 nM, about 90 nM, about 95 nM, or about 100 nM. An IC₅₀ of ++ refers to an IC₅₀ between 100 nM and 1 μM. In embodiments described herein, ++ refers to IC₅₀(s) of about 101 nM, about 200 nM, about 300 nM, about 400 nM, about 500 nM, about 600 nM, about 700 nM, about 800 nM, about 900 nM, or about 1 μM. An IC₅₀ of + refers to an IC₅₀ between 1 μM and 30 μM. In embodiments described herein, + refers to IC₅₀(s) of about 1.1 μM, about 2 μM, about 3 μM, about 4 μM, about 5 μM, about 10 μM, about 15 μM, about 20 μM, about 25 μM, or about 30 μM. An IC₅₀ of − refers to an inactive compound.

In certain embodiments described herein, the compounds of Formula I or pharmaceutically acceptable salts thereof, display selectivity for inhibition of TYRO3 protein kinase. In some embodiments, the compounds of Formula I or pharmaceutically acceptable salts thereof, are at least 2% more selective for inhibiting TYRO3 over AXL or

Mer protein kinases. In some embodiments, the compounds of Formula I or pharmaceutically acceptable salts thereof, are at least 3%, at least 4%, at least 5% at least 6%, at least 7%, at least 8%, at least 9%, at least 10%, at least 15%, or at least 20% more selective for inhibiting TYRO3 over AXL or Mer protein kinases.

### IV. Pharmaceutical Compositions

In certain embodiments, described herein are pharmaceutical compositions comprising at least one compound of Formula I as described herein, and a pharmaceutically acceptable excipient or carrier.

A “pharmaceutically acceptable excipient” refers to a vehicle for containing a functionalized cell or an acellular extracellular matrix that can be introduced into a subject without significant adverse effects and without having deleterious effects on the functionalized cell or acellular extracellular matrix. That is, “pharmaceutically acceptable” in the context of a formulation refers to any formulation which is safe and provides the appropriate delivery for the desired route of administration of an effective amount of at least one functionalized cell or acellular extracellular matrix for use in the methods disclosed herein. Pharmaceutically acceptable carriers or vehicles or excipients are well known. Descriptions of suitable pharmaceutically acceptable carriers, and factors involved in their selection, are found in a variety of readily available sources such as, for example, Remington's Pharmaceutical Sciences, 18th ed., 1990, herein incorporated by reference in its entirety for all purposes. Such carriers can be suitable for any route of administration (e.g., parenteral, enteral (e.g., oral), or topical application). Such pharmaceutical compositions can be buffered, for example, wherein the pH is maintained at a particular desired value, ranging from pH 4.0 to pH 9.0, in accordance with the stability of the functionalized cell or acellular extracellular matrix and route of administration.

Suitable pharmaceutically acceptable carriers include, for example, sterile water, salt solutions such as saline, glucose, buffered solutions such as phosphate buffered solutions or bicarbonate buffered solutions, alcohols, gum arabic, vegetable oils, benzyl alcohols, polyethylene glycols, gelatine, carbohydrates (e.g., lactose, amylose or starch), magnesium stearate, talc, silicic acid, viscous paraffin, white paraffin, glycerol, alginates, hyaluronic acid, collagen, perfume oil, fatty acid monoglycerides and diglycerides, pentaerythritol fatty acid esters, hydroxy methylcellulose, polyvinyl pyrrolidone, and the like. Pharmaceutical compositions or vaccines may also include auxiliary agents including, for example, diluents, stabilizers (e.g., sugars and amino acids), preservatives, wetting agents, emulsifiers, pH buffering agents, viscosity enhancing additives, lubricants, salts for influencing osmotic pressure, buffers, vitamins, coloring, flavoring, aromatic substances, and the like which do not deleteriously react with a functionalized cell or an acellular extracellular matrix.

For liquid formulations, for example, pharmaceutically acceptable carriers may be aqueous or non-aqueous solutions, suspensions, emulsions, or oils. Non-aqueous solvents include, for example, propylene glycol, polyethylene glycol, and injectable organic esters such as ethyl oleate. Aqueous carriers include, for example, water, alcoholic/aqueous solutions, emulsions or suspensions, including saline and buffered media. Examples of oils include those of petroleum, animal, vegetable, or synthetic origin, such as peanut oil, soybean oil, mineral oil, olive oil, sunflower oil, and fish-liver oil. Solid carriers/diluents include, for example, a gum, a starch (e.g., corn starch, pregeletanized starch), a sugar (e.g., lactose, mannitol, sucrose, or dextrose), a cellulosic material (e.g., microcrystalline cellulose), an acrylate (e.g., polymethylacrylate), calcium carbonate, magnesium oxide, talc, or mixtures thereof.

Optionally, sustained or directed release pharmaceutical compositions or vaccines can be formulated. This can be accomplished, for example, through use of liposomes or compositions wherein the active compound is protected with differentially degradable coatings (e.g., by microencapsulation, multiple coatings, and so forth). Such compositions may be formulated for immediate or slow release. It is also possible to freeze-dry the compositions and use the lyophilisates obtained (e.g., for the preparation of products for injection).

### V. Therapeutic Methods

In certain embodiments, the subject matter described herein is directed to methods of treating disorders associated with TAM receptor tyrosine kinases, the method comprising the step of administering to a subject an effective amount of at least one compound of Formula A.

In certain embodiments, the method comprises treatment of a disorder associated with TYRO3 or Mer tyrosine kinases.

In certain embodiments, the method comprises treatment of a disorder associated with TYRO3 and Mer tyrosine kinases.

In certain embodiments, the method comprises treatment of a disorder associated with TYRO3 tyrosine kinases.

In certain embodiments described herein, the disorder is a cancer, an infection, a fibrosis, a thrombotic disorder, a clotting disorder, or a disorder associated with an immunosuppressed microenvironment surrounding diseased tissue.

In certain embodiments, the disorder is cancer.

In certain embodiments, the cancer is selected from the group consisting of breast cancer, cervical cancer, gastrointestinal cancer, colorectal cancer, brain cancer, skin cancer, prostate cancer, ovarian cancer, thyroid cancer, testicular cancer, pancreatic cancer, liver cancer, bladder cancer, hematologic cancer, endometrial cancer, melanoma, glioma, leukemia, lymphoma, chronic myeloproliferative disorder, myelodysplastic syndrome, myeloproliferative neoplasm, plasma cell neoplasm (myeloma), myeloid leukemia.

In certain embodiments, the cancer is TYRO3 and/or MerTK +/+.

In certain embodiments, the cancer is TYRO3 and/or MerTK −/−.

In embodiments described herein, the disorder is a thrombotic disorder or a clotting disorder.

In certain embodiments, the thrombotic disorder or clotting disorder involves ischemic heart disease, stroke, or acute myocardial infarction.

In certain embodiments described herein, the disorder is an infection. In certain aspects of these embodiments, the infection is viral. In certain aspects of these embodiments, the infection is bacterial.

In certain embodiments described herein, the disorder is fibrosis.

In certain embodiments, the method of treatment further comprises administering an additional active agent, such as an active agent useful in the treatment of cancer. The additional compounds may optionally be administered concurrently. As used herein, the word “concurrently” means sufficiently close in time to produce a combined effect (that is, concurrently may be simultaneously, or it may be two or more events occurring within a short time period before or after each other).

Compounds of Formula A may optionally be administered in conjunction with other compounds useful in the treatment of blood clot formation in a subject in need thereof (e.g., a subject afflicted with coronary artery disease, peripheral vascular disease, or cerebrovascular disease, or prior to any medical or surgical procedure in which diminished coagulation potential is desirable, a non-limiting example of which is pulmonary vein ablation).

The present subject matter is primarily concerned with the treatment of human subjects, but the invention may also be carried out on animal subjects, particularly mammalian subjects such as mice, rats, dogs, cats, livestock and horses for veterinary purposes, and for drug screening and drug development purposes. Subjects may be of any age, including infant, juvenile, adolescent, adult, and geriatric subjects.

As noted above, the subject matter provides pharmaceutical formulations comprising the compounds of Formula A (including the pharmaceutically acceptable salts thereof), in pharmaceutically acceptable carriers for oral, rectal, topical, buccal, parenteral, intramuscular, intradermal, or intravenous, and transdermal administration.

The therapeutically effective dosage of any specific compound can vary somewhat from compound to compound, and patient to patient, and will depend upon the condition of the patient and the route of delivery. As a general proposition, a dosage from about 0.1 to about 50 mg/kg will have therapeutic efficacy, with all weights being calculated based upon the weight of the active compound, including the cases where a salt is employed. Toxicity concerns at the higher level may restrict intravenous dosages to a lower level such as up to about 10 mg/kg, with all weights being calculated based upon the weight of the active base, including the cases where a salt is employed. A dosage from about 10 mg/kg to about 50 mg/kg may be employed for oral administration. In some embodiments, a dosage from about 0.5 mg/kg to 5 mg/kg may be employed for intramuscular injection. In some embodiments, dosages are 1 μmol/kg to 50 μmol/kg, and more preferably 22 μmol/kg and 33 μmol/kg of the compound for intravenous or oral administration. The duration of the treatment can be once per day for a period of two to three weeks or until the condition is essentially controlled.

In certain embodiments, the subject matter described herein is directed to methods of inhibiting TAM receptor kinases, in at least one cell, the method comprising the step of contacting the at least once cell with an effective amount of at least one compound of Formula A. In certain aspects of these embodiments, the method comprises inhibiting TYRO3 or Mer tyrosine kinases. In certain aspects of these embodiments, the method comprises inhibiting TYRO3 and Mer tyrosine kinases. In certain aspects of these embodiments, the method comprises inhibiting TYRO3 tyrosine kinase alone.

In embodiments described herein, the cell has been isolated from a mammal prior to the contacting step.

In embodiments described herein, the mammal has been diagnosed with a need for treatment of a disorder related to the TAM receptor tyrosine kinases, prior to the administering step.

In certain embodiments, the mammal has been diagnosed with a need for treatment of a disorder related to TYRO3 or Mer tyrosine kinases, prior to the administering step.

In certain embodiments, the mammal has been diagnosed with a need for treatment of a disorder related to TYRO3 and Mer tyrosine kinases, prior to the administering step.

In certain embodiments, the mammal has been diagnosed with a need for treatment of a disorder related to TYRO3 tyrosine kinase, prior to the administering step.

The disclosed subject matter is further described in the following non-limiting Examples. It should be understood that these Examples, while indicating preferred embodiments of the invention, are given by way of illustration only.

### EXAMPLES

### Example 1—Syntheses trans-4-(2-((3,5-Dichlorophenyl)amino)-5-(4-((4-methylpiperazin-1-yl)methyl)phenyl)-7H-pyrrolo[2,3-d]pyrimidin-7-yl)cyclohexan-1-ol

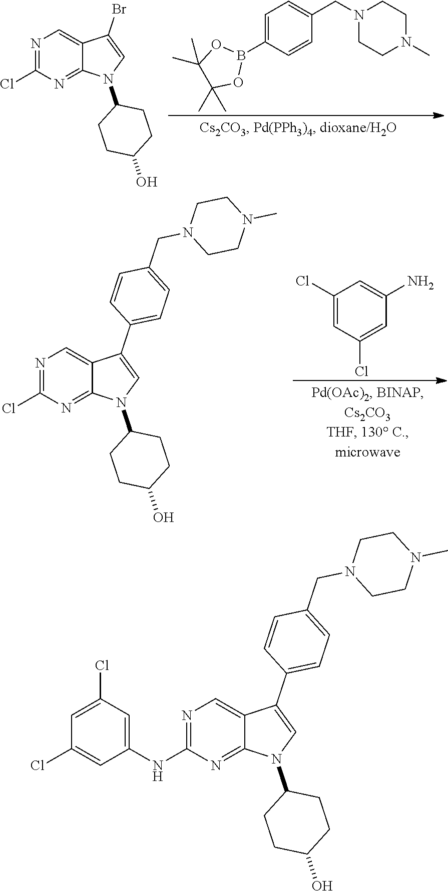

To a solution of trans-4-(5-bromo-2-chloro-7H-pyrrolo[2,3-d]pyrimidin-7-yl)cyclohexan-1-ol (15.0 g, 45.4 mmol) in 1,4-dioxane (150.0 mL) and water (10.0 mL) were added 1-methyl-4-(4-(4,4,5,5-tetramethyl-1,3,2-dioxaborolan-2-yl)-benzyl) piperazine (15.1 g, 47.6 mmol), cesium carbonate (22.2 g, 68.1 mmol), and Pd(PPh₃)₄ (1.57 g, 1.36 mmol). The reaction mixture was heated under nitrogen atmosphere at 90° C. for 12 h. Then the reaction mixture was cooled to room temperature, quenched with H₂O, extracted with EtOAc (3×). The combined organic layer was washed with brine, dried (MgSO₄), and concentrated under reduced pressure. The residue was purified by an ISCO reversed column (methanol/water+0.1% HCl, 10-100%) to afford a slight yellow solid, which was triturated with methanol. The solid was filtered to afford the desired product tert-butyl 4-(2-amino-4-((trans-4-hydroxycyclohexyl)amino)pyrimidin-5-yl)-5,6-dihydropyridine-1 (2H)-carboxylate (13.1 g, 65%) as a yellow solid.

¹H NMR (400 MHz, dmso) δ 10.48 (s, 1H), 9.24 (s, 1H), 8.25 (s, 1H), 7.77 (d, J=7.7 Hz, 2H), 7.42 (s, 2H), 4.80 (s, 1H), 4.64-4.53 (m, 1H), 3.65-3.52 (m, 3H), 3.16 (s, 2H), 3.00 (s, 5H), 2.72 (s, 3H), 2.03-1.89 (m, 6H), 1.43 (q, J=11.2 Hz, 2H). ¹³C NMR (100 MHz, CD₃OD) δ 154.0, 153.2, 151.4, 137.2, 133.5, 131.5, 127.8, 126.1, 117.4, 117.3, 70.1, 63.4, 55.6, 54.6, 53.4, 45.9, 35.2, 31.6. MS (ESI) m/z calculated for C24H31ClN5O: 440.22 [M+H]+; found 440.25. LC-MS: >95% purity.

To a solution of tert-butyl 4-(2-amino-4-((trans-4-hydroxycyclohexyl)amino)-pyrimidin-5-yl)-5,6-dihydropyridine-1 (2H)-carboxylate (400.0 mg, 0.91 mmol) and 3,5-dichloroaniline (294.6 mg, 1.82 mmol) in dry THF (10 mL) were added palladium (II) acetate (60.69 mg, 272.2 μmol), BINAP (169.8 mg, 272.2 μmol), and Cs₂CO₃ (444.3 mg, 1.36 mmol). The reaction mixture was heated under nitrogen atmosphere and microwave radiation at 130° C. for 1.5 h, then quenched by water. The mixture was extracted with EtOAc (3×15 mL). The combined organic layer was washed with water and brine, dried (Na₂SO₄), and concentrated. The residue was purified by an ISCO reversed column (methanol/water+0.1% HCl, 10-100%) to afford crude product which was recrystallized from methanol to afford the title compound (210 mg, 0.91 mmol, 40.8%) as a yellow solid.

¹H NMR (850 MHz, DMSO-d₆) δ 10.07 (s, 1H), 9.19 (s, 1H), 8.03 (d, J=1.9 Hz, 2H), 7.99 (s, 1H), 7.87 (d, J=7.7 Hz, 2H), 7.68 (s, 2H), 7.09 (t, J=1.9 Hz, 1H), 4.51 (tt, J=11.0, 4.9 Hz, 2H), 4.40 (s, 2H), 3.68-3.56 (m, 5H), 3.49-3.29 (m, 3H), 2.80 (s, 3H), 2.06-2.00 (m, 6H), 1.43 (m, 2H). MS (ESI) m/z calculated for C30H34C12N6O: 565.22 [M+H]+; found 565.20. LC-MS: >96% purity.

Additional compounds were prepared following the synthesis procedure described in Example 1 using the corresponding reagents.

Table 2 describes compounds prepared following procedures described in Example 1 using appropriate reagents. Biological assays were performed as described in Example 5 (Note: Mer IC₅₀: ++++ means <10 nM; +++ means between 10-100 nM, + means between 100 nM-1 μM; + means between 1-30 μM; − means inactive).

TABLE 2

| | | | | | Physical Data |
|---|---|---|---|---|---|
| Cpd. | | TYRO3 | MERTK | AXL | MS m/z (M + 1) or/and ¹H |
| No. | Structure | IC₅₀ | IC₅₀ | IC₅₀ | NMR |
| 1 | 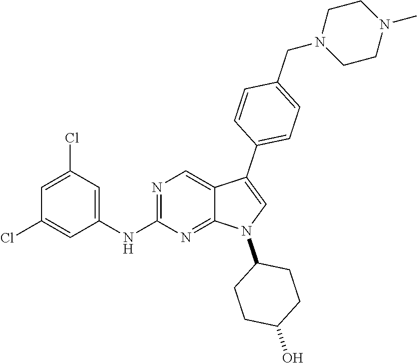 | ++++ | +++ | +++ | ¹H NMR (850 MHz, DMSO-d₆) δ 10.07 (s, 1H), 9.19 (s, 1H), 8.03 (d, J = 1.9 Hz, 2H), 7.99 (s, 1H), 7.87 (d, J = 7.7 Hz, 2H), 7.68 (s, 2H), 7.09 (t, J = 1.9 Hz, 1H), 4.51 (tt, J = 11.0, 4.9 Hz, 2H), 4.40 (s, 2H), 3.68-3.56 (m, 5H), 3.49-3.29 (m, 3H), 2.80 (s, 3H), 2.06-2.00 (m, 6H), 1.43 (m, 2H). MS (ESI) m/z calcd for C₃₀H₃₅Cl₂N₆O⁺: 565.22 [M + H]⁺; found m/z 565.20. |
| 2 | 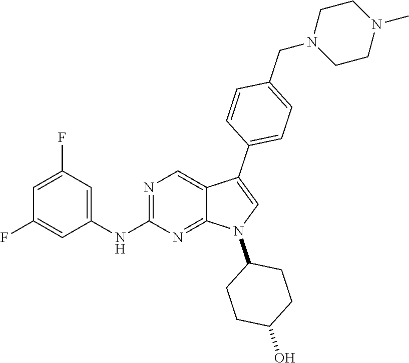 | ++++ | ++++ | +++ | ¹H NMR (850 MHz, DMSO-d₆) δ 10.01 (s, 1H), 9.15 (s, 1H), 7.94 (s, 1H), 7.84 (d, J = 7.8 Hz, 2H), 7.65 (dd, J = 10.8, 2.4 Hz, 2H), 7.58-7.57 (m, 1H), 6.72-6.98 (m, 1H), 4.48-3.53 (m, 2H), 4.25 (s, 2H), 3.60-3.55 (m, 5H), 3.51- 3.39 (m, 3H), 2.82 (s, 3H), 2.05- 1.98 (m, 8H), 1.42-1.38 (m, 2H). MS (ESI) m/z calcd for C₃₀H₃₅F₂N₆O⁺: 533.28 [M + H]⁺; 533.30. |
| 3 | 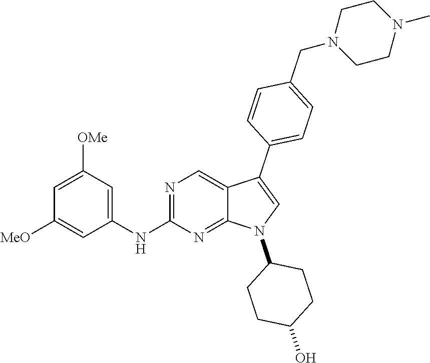 | + | ++ | ++ | ¹H NMR (850 MHz, DMSO- d₆) δ 9.24 (s, 1H), 8.13 (s, 1H), 7.86 (d, J = 7.8 Hz, 2H), 7.74 (d, J = 7.8 Hz, 2H), 7.11 (d, J = 2.3 Hz, 2H), 6.21 (t, J = 2.2 Hz, 1H), 4.55 (tt, J = 11.6, 4.2 Hz, 2H), 4.42 (s, 2H), 3.78 (s, 6H), 3.68-3.63 (m, 2H), 3.60-3.56 (m, 2H), 3.53- 3.48 (m, 2H), 3.43-3.36 (m, 2H), 2.80 (s, 3H), 2.05-1.99 (m, 6H), 1.99-1.96 (m, 2H), 1.38-1.29 (m, 2H). MS (ESI) m/z calcd for C₃₂H₄₁N₆O₃⁺: 557.32 [M + H]⁺; found m/z 557.20. |
| 4 | 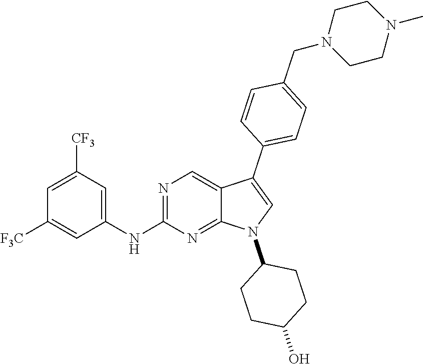 | ++++ | ++ | ++ | ¹H NMR (400 MHz, D₂O) δ 8.57 (s, 1H), 7.93 (s, 2H), 7.60 (s, 1H), 7.54 (s, 1H), 7.46 (d, J = 7.9 Hz, 2H), 7.40 (d, J = 7.8 Hz, 2H), 4.31 (s, 2H), 4.08-3.99 (m, 2H), 3.78-3.50 (m, 9H), 3.05 (s, 3H), 1.97 (s, 2H), 1.83-1.74 (m, 4H), 1.73-1.62 (m, 2H), 1.33- 1.18 (m, 2H). MS (ESI) m/z calcd for C₃₂H₃₅F₆N₆O⁺: 633.27 [M + H]⁺; found m/z 633.30. |
| 5 | 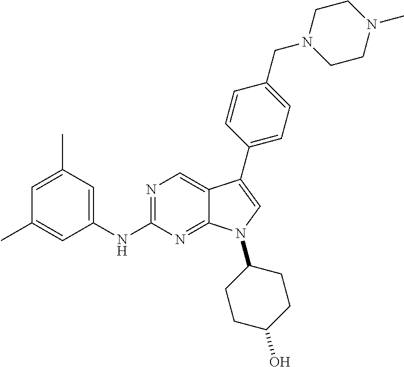 | ++++ | ++ | ++ | ¹H NMR (850 MHz, DMSO-d₆) δ 9.17 (s, 1H), 8.01 (s, 1H), 7.86-7.81 (m, 2H), 7.70-7.64 (m, 2H), 7.48-7.43 (m, 2H), 6.67 (s,1H). 4.50-4.47 (m, 2H), 4.36 (s, 2H), 3.64 (s, 2H), 3.57- 3.48 (m, 8H), 2.82 (s, 3H), 2.28 (s, 6H), 2.04-2.01 (m, 8H), 1.42-1.39 (m, 2H). MS (ESI) m/z calcd for C₃₂H₄₁N₆O⁺: 525.33 [M + H]⁺; found m/z 525.30. |
| 6 | 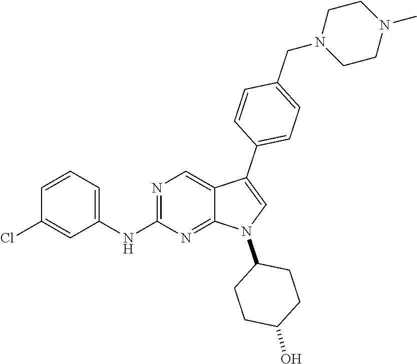 | ++++ | ++++ | +++ | ¹H NMR (850 MHz, DMSO) δ 9.98 (s, 1H), 9.18 (s, 1H), 8.30 (t, J = 2.1 Hz, 1H), 8.00 (s, 1H), 7.86 (d, J = 7.8 Hz, 2H), 7.70 (d, J = 7.7 Hz, 2H), 7.58 (ddd, J = 8.3, 2.1, 0.9 Hz, 1H), 7.32 (t, J = 8.0 Hz, 1H), 4.54 - 4.49 (m, 1H), 4.40 (s, 2H), 3.68-3.57 (m, 5H), 3.41 (d, J = 83.1 Hz, 4H), 2.80 (s, 3H), 2.07-1.98 (m, 8H), 1.48-1.38 (m, 2H). MS (ESI) m/z calcd for C₃₀H₃₆ClN₆O⁺: 531.26 [M + H]⁺; found m/z 531.30. |
| 7 | 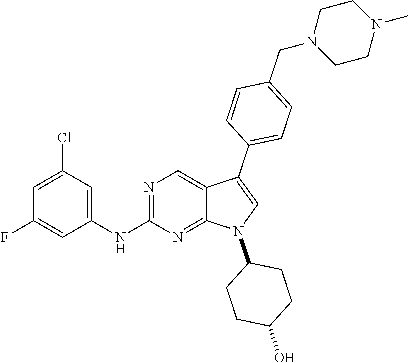 | ++++ | ++++ | +++ | ¹H NMR (400 MHz, D₂O) δ 8.60 (s, 1H), 7.68 (s, 1H), 7.53 (d, J = 8.0 Hz, 2H), 7.45 (d, J = 8.0 Hz, 2H), 7.40 (s, 1H), 7.04 (d, J = 11.0 Hz, 1H), 6.78 (td, J = 8.5, 2.2 Hz, 1H), 4.35 (s, 2H), 4.12-4.00 (m, 1H), 3.79-3.52 (m, 10H), 3.05 (s, 3H), 2.05 (d, J = 12.1 Hz, 2H), 1.93 (d, J = 12.3 Hz, 2H), 1.81 (q, J = 12.4 Hz, 2H), 1.36 (q, J = 12.2 Hz, 2H). MS (ESI) m/z calcd for C₃₀H₃₅ClFN₆O⁺: 549.25 [M + H]⁺; found m/z 549.30. |
| 8 | 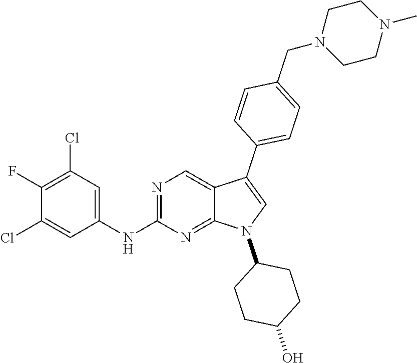 | ++++ | +++ | +++ | ¹H NMR (400 MHz, DMSO-d₆) δ 10.00 (s, 1H), 9.17 (s, 1H), 8.12 (d, J = 6.1 Hz, 2H), 7.98 (s, 1H), 7.86 (d, J = 7.9 Hz, 2H), 7.65 (d, J = 7.9 Hz, 2H), 4.56-4.45 (m, 1H), 4.37 (s, 2H), 3.69- 3.58 (m, 9H), 2.83 (s, 3H), 2.08- 1.97 (m, 6H), 1.50-1.34 (m, 2H). MS (ESI) m/z calcd for C₃₀H₃₄Cl₂FN₆O⁺: 583.21 [M + H]⁺; found m/z 583.10. |
| 9 | 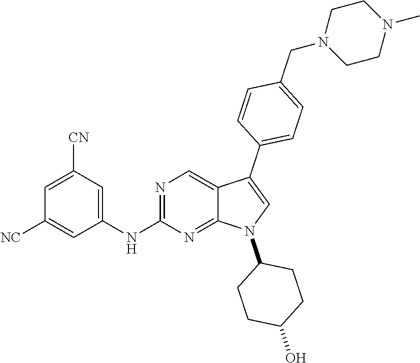 | ++++ | +++ | +++ | ¹H NMR (400 MHz, DMSO-d₆) δ 10.30 (s, 1H), 9.16 (s, 1H), 8.58 (s, 2H), 7.91 (d, J = 5.9 Hz, 2H), 7.71 (d, J = 7.9 Hz, 2H), 7.34 (d, J = 7.9 Hz, 2H), 3.65-3.54 (m, 2H), 3.46 (s, 2H), 2.44-2.22 (m, 9H), 2.15 (s, 3H), 2.06- 1.96 (m, 6H), 1.48-1.37 (m, 2H). MS (ESI) m/z calcd for C₃₂H₃₅N₈O⁺: 547.29 [M + H]⁺; found m/z 547.30. |
| 10 | 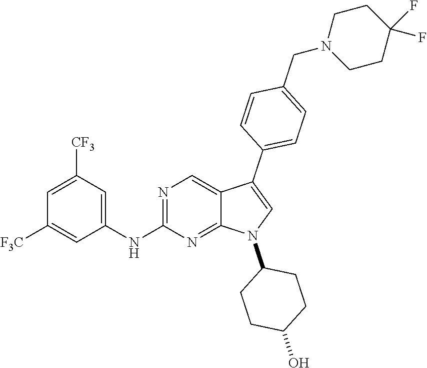 | +++ | ++ | ++ | ¹H NMR (400 MHz, MeOD) δ 9.09 (s, 1H), 8.48 (s, 2H), 7.95 (s, 1H), 7.87 (d, J = 7.9 Hz, 2H), 7.69 (d, J = 8.0 Hz, 2H), 7.62 (s, 1H), 4.73-4.63 (m, 1H), 4.47 (s, 2H), 3.80-3.70 (m, 1H), 3.69- 3.60 (m, 2H), 3.40-3.34 (m, 2H), 2.49-2.32 (m, 4H), 2.21-2.01 (m, 8H), 1.63-1.49 (m, 2H). MS (ESI) m/z calcd for C₃₂H₃₂F₈N₅O⁺: 654.24 [M + H]⁺; found m/z 654.20. |
| 11 | 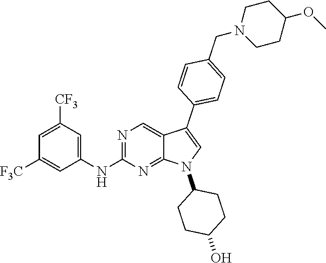 | ++++ | ++ | ++ | ¹H NMR (850 MHz, MeOD) δ 9.11 (s, 1H), 8.46 (s, 2H), 8.05 (s, 1H), 7.88- 7.84 (m, 2H), 7.71 (s, 1H), 7.68-7.64 (m, 2H), 4.69-4.65 (m, 1H), 4.38 (s, 2H), 3.76-3.71 (m, 1H), 3.65-3.62 (m, 1H), 3.37 (s, 3H), 3.35-3.32 (m, 2H), 3.28-3.24 (m, 2H), 2.19-2.05 (m, 9H), 1.96-1.91 (m, 1H), 1.58- 1.52 (m, 2H). MS (ESI) m/z calcd for C₃₂H₃₆F₆N₅O₂⁺: 648.27 [M + H]⁺; found m/z 648.20. |
| 12 | 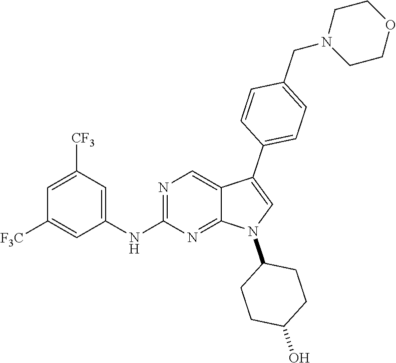 | ++++ | + | ++ | ¹H NMR (850 MHz, CD₃OD) δ 9.12 (d, J = 3.3 Hz, 1H), 8.45 (s, 2H), 8.06 (s, 1H), 7.87 (d, J = 8.2 Hz, 2H), 7.72- 7.68 (m, 2H), 7.65 (d, J = 8.3 Hz, 1H), 4.70-4.64 (m, 1H), 4.41 (s, 2H), 4.07 (dd, J = 13.4, 4.1 Hz, 1H), 3.83- 3.78 (m, 2H), 3.74 (tt, J = 11.2, 4.3 Hz, 1H), 3.43 (d, J = 12.3 Hz, 1H), 3.26 (td, J = 12.7, 3.9 Hz, 1H), 2.90 (m, 4H), 2.19-2.05 (m, 6H), 1.58-1.52 (m, 2H). MS (ESI) m/z calcd for C₃₁H₃₂F₆N₅O₂⁺: 620.24 [M + H]⁺; found m/z 620.20. |
| 13 | 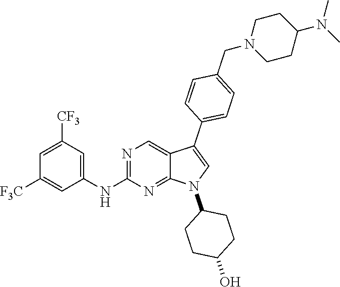 | ++++ | ++ | ++ | ¹H NMR (850 MHz, CD₃OD) δ 9.13 (s, 1H), 8.44 (s, 2H), 8.09 (d, J = 4.7 Hz, 1H), 7.89-7.85 (m, 2H), 7.75- 7.70 (m, 3H), 4.69-4.64 (m, 1H), 4.41 (s, 2H), 3.76-3.68 (m, 4H), 3.66- 3.60 (m, 1H), 3.27-3.21 (m, 1H), 2.91 (d, J = 15.4 Hz, 6H), 2.42-2.37 (m, 4H), 2.24-2.06 (m, 8H), 1.57- 1.51 (m, 2H). MS (ESI) m/z calcd for C₃₄H₃₉F₆N₆O⁺: 661.30 [M + H]⁺; found m/z 661.30. |
| 14 | 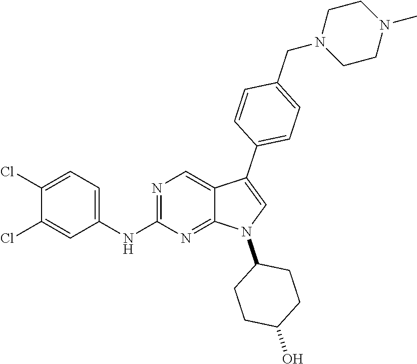 | ++++ | ++++ | ++++ | ¹H NMR (850 MHz, DMSO-d₆) δ 9.98 (s, 1H), 9.17 (s, 1H), 8.51 (d, J = 2.5 Hz, 1H), 7.97 (s, 1H), 7.88-7.84 (m, 2H), 7.66 (s, 1H), 7.63 (dd, J = 8.9, 2.5 Hz, 2H), 7.53 (d, J = 8.8 Hz, 1H), 4.55- 4.49 (m, 1H), 4.39 (s, 2H), 3.72- 3.59 (m, 9H), 2.84-2.78 (m, 4H), 2.05-1.99 (m, 6H), 1.44 (qd, J = 13.4, 3.8 Hz, 2H). MS (ESI) m/z calcd for C₃₀H₃₅Cl₂N₆O⁺: 564.22 [M + H]⁺; found m/z 565.30. |
| 15 | 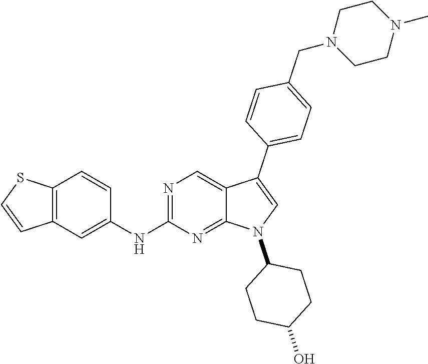 | +++ | +++ | +++ | ¹H NMR (850 MHz, DMSO-d₆) δ 9.20 (s, 1H), 8.57 (s, 1H), 8.03 (s, 1H), 7.94 (d, J = 8.6 Hz, 1H), 7.86 (d, J = 7.6 Hz, 2H), 7.78 (d, J = 5.3 Hz, 1H), 7.70 (s, 1H), 7.65 (d, J = 8.8 Hz, 1H), 7.38 (d, J = 5.3 Hz, 1H), 4.58-4.49 (m, 1H), 4.39 (s, 2H), 3.70-3.52 (m, 11H), 2.81 (s, 3H), 2.07-2.00 (m, 6H), 1.45 (d, J = 12.4 Hz, 2H). MS (ESI) m/z calcd for C₃₂H₃₇N₆OS⁺: 553.27 [M + H]⁺; found m/z 553.20. |
| 18 | 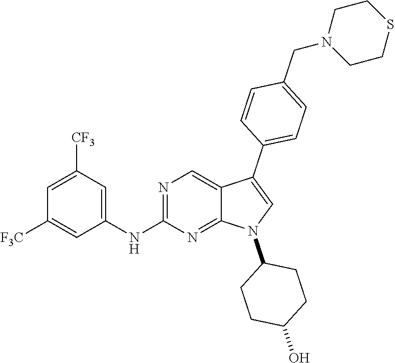 | ++++ | + | + | ¹H NMR (850 MHz, CD₃OD) δ 9.12 (s, 1H), 8.46 (s, 2H), 8.04 (s, 1H), 7.87 (d, J = 8.3 Hz, 2H), 7.70 (s, 1H), 7.68 (d, J = 8.3 Hz, 2H), 4.67 (tt, J = 11.9, 4.1 Hz, 1H), 4.43 (s, 2H), 3.79 (d, J = 13.2 Hz, 2H), 3.74 (tt, J = 11.0, 4.3 Hz, 1H), 3.30-3.27 (m, 3H), 3.16 (t, J = 12.4 Hz, 2H), 2.87 (d, J = 14.8 Hz, 2H), 2.18-2.12 (m, 5H), 2.08 (qd, J = 12.7, 3.3 Hz, 2H), 1.58-1.52 (m, 2H). MS (ESI) m/z calcd for C₃₁H₃₂F₆N₅OS⁺: 536.22 [M + H]⁺; found m/z 536.20. |
| 19 | 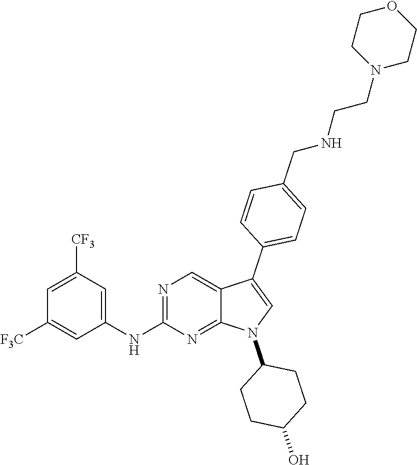 | ++++ | ++ | ++ | ¹H NMR (850 MHz, CD₃OD) δ 9.11 (s, 1H), 8.45 (s, 2H), 8.06 (s, 1H), 7.84 (d, J = 8.2 Hz, 2H), 7.77-7.72 (m, 3H), 4.66 (ddd, J = 12.1, 8.0, 4.2 Hz, 1H), 4.37 (s, 2H), 4.12-3.92 (m, 3H), 3.76-3.71 (m, 2H), 3.68 (t, J = 6.4 Hz, 3H), 3.61 (s, 1H), 3.28-3.13 (m, 4H), 2.20-2.12 (m, 6H), 2.08 (qd, J = 12.7, 3.4 Hz, 2H), 1.59-1.51 (m, 2H). MS (ESI) m/z calcd for C₃₃H₃₇F₆N₆O₂⁺: 663.28 [M + H]⁺; found m/z 663.30. |
| 20 | 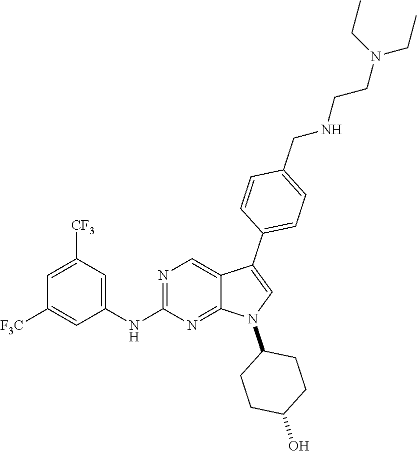 | ++++ | ++ | ++ | ¹H NMR (850 MHz, CD₃OD) δ 9.12 (s, 1H), 8.44 (s, 2H), 8.09 (s, 1H), 7.84 (d, J = 8.3 Hz, 2H), 7.77-7.73 (m, 3H), 4.66 (tt, J = 11.9, 4.2 Hz, 1H), 4.38 (s, 2H), 3.73 (tt, J = 11.1, 4.3 Hz, 2H), 3.66 (t, J = 6.7 Hz, 2H), 3.60 (t, J = 7.3 Hz, 1H), 3.35 (dd, J = 7.3, 3.3 Hz, 3H), 2.18-2.06 (m, 10H), 1.57- 1.51 (m, 2H), 1.41 (t, J = 7.3 Hz, 6H). MS (ESI) m/z calcd for C₃₃H₃₉F₆N₆O⁺: 649.30 [M + H]⁺; found m/z 649.30. |
| 21 | 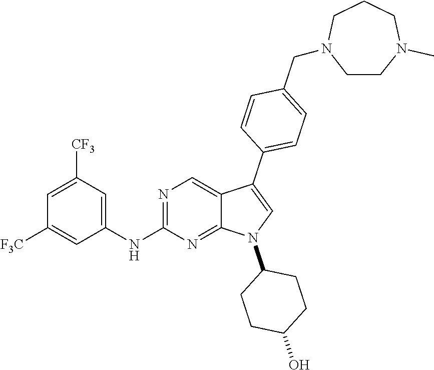 | ++++ | ++ | ++ | ¹H NMR (400 MHz, CD₃OD) δ 9.14 (s, 1H), 8.44 (s, 2H), 8.11 (s, 1H), 7.87 (d, J = 8.5 Hz, 2H), 7.78 (d, J = 8.4 Hz, 2H), 7.74 (s, 1H), 4.70-4.61 (m, 1H), 4.56 (s, 2H), 3.99-3.83 (m, 4H), 3.78- 3.69 (m, 4H), 3.66-3.41 (m, 3H), 3.00 (s, 3H), 2.46-2.32 (m, 3H), 2.23- 2.02 (m, 6H), 1.61-1.47 (m, 2H). MS (ESI) m/z calcd for C₃₃H₃₆F₆N₆O⁺: 647.29 [M + H]⁺; found m/z 647.30. |
| 22 | 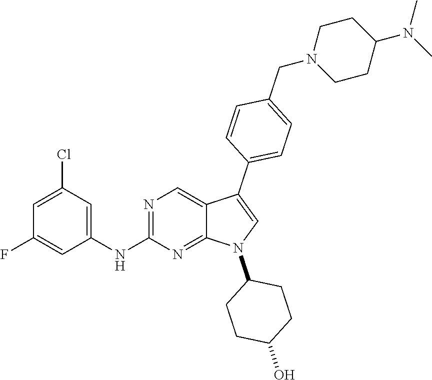 | ++++ | ++++ | +++ | ¹H NMR (850 MHz, CD₃OD) δ 9.08 (s, 1H), 8.07 (s, 1H), 7.85 (d, J = 8.3 Hz, 2H), 7.76 (s, 1H), 7.74 (d, J = 8.2 Hz, 2H), 7.49 (dt, J = 10.5, 2.1 Hz, 1H), 7.04 (dt, J = 8.4, 2.1 Hz, 1H), 4.65-4.58 (m, 1H), 4.43 (s, 2H), 3.77 (tt, J = 11.2, 4.2 Hz, 2H), 3.70 (d, J = 13.1 Hz, 2H), 3.64 (tt, J = 12.1, 3.9 Hz, 1H), 3.25 (t, J = 13.1 Hz, 2H), 2.92 (s, 6H), 2.40 (d, J = 13.9 Hz, 2H), 2.23- 2.13 (m, 8H), 1.60-1.53 (m, 2H). MS (ESI) m/z calcd for C₃₂H₃₉ClFN₆O⁺: 577.28 [M + H]⁺; found m/z 577.29. |
| 23 | 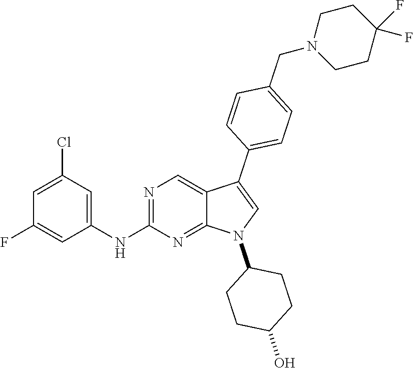 | ++++ | +++ | ++ | ¹H NMR (850 MHz, CD₃OD) δ 9.07 (s, 1H), 8.05 (s, 1H), 7.86 (d, J = 8.2 Hz, 2H), 7.76 (s, 1H), 7.71 (d, J = 8.2 Hz, 2H), 7.50 (dt, J = 10.6, 2.1 Hz, 1H), 7.03 (dt, J = 8.3, 2.1 Hz, 1H), 4.62 (tt, J = 10.7, 5.3 Hz, 1H), 4.47 (s, 2H), 3.77 (tt, J = 11.2, 4.2 Hz, 1H), 3.65 (d, J = 12.9 Hz, 2H), 3.37-3.32 (m, 2H), 2.45-2.35 (m, 6H), 2.20- 2.14 (m, 6H), 1.60-1.53 (m, 2H). MS (ESI) m/z calcd for C₃₀H₃₂ClF₃N₅O⁺: 570.22 [M + H]⁺; found m/z 570.20. |
| 24 | 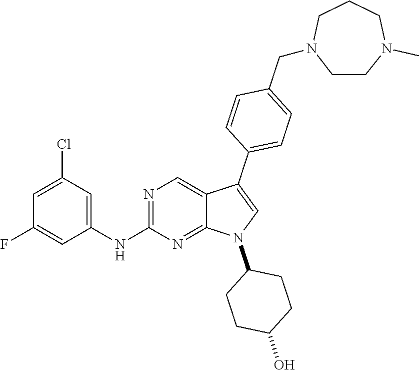 | ++++ | ++++ | +++ | ¹H NMR (850 MHz, CD₃OD) δ 9.07 (s, 1H), 8.05 (s, 1H), 7.85 (d, J = 8.2 Hz, 2H), 7.78-7.75 (m, 3H), 7.50 (dt, J = 10.6, 2.1 Hz, 1H), 7.03 (dt, J = 8.3, 2.1 Hz, 1H), 4.64-4.59 (m, 1H), 4.54 (s, 2H), 3.99-3.40 (m, 8H), 3.00 (s, 3H), 2.45-2.31 (m, 2H), 2.21-2.13 (m, 9H), 1.61-1.53 (m, 2H). MS (ESI) m/z calcd for C₃₁H₃₇ClFN6O⁺: 563.26 [M + H]⁺; found m/z 563.30. |
| 25 | 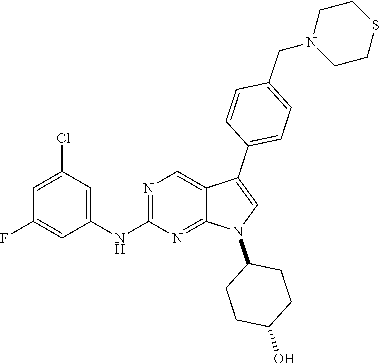 | ++++ | ++ | ++ | ¹H NMR (850 MHz, CD₃OD) δ 9.09 (s, 1H), 8.08 (s, 1H), 7.87 (d, J = 8.2 Hz, 2H), 7.78 (s, 1H), 7.71 (d, J = 8.2 Hz, 2H), 7.52 (dt, J = 10.5, 2.1 Hz, 1H), 7.06 (dt, J = 8.4, 2.0 Hz, 1H), 4.66-4.61 (m, 1H), 4.45 (s, 2H), 3.83- 3.76 (m, 3H), 3.32 - 3.28 (m, 2H), 3.19 (t, J = 13.2 Hz, 2H), 2.89 (d, J = 16.4 Hz, 2H), 2.23-2.15 (m, 8H), 1.63-1.53 (m, 2H). MS (ESI) m/z calcd for C₂₉H₃₂ClFN₆OS⁺: 552.19 [M + H]⁺; found m/z 552.20. |
| 26 | 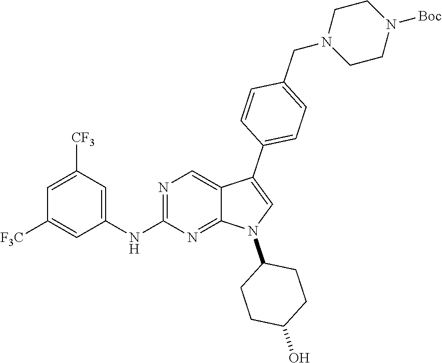 | +++ | + | + | ¹H NMR (850 MHz, CD₃OD) δ 9.09 (s, 1H), 8.49 (s, 2H), 7.96 (s, 1H), 7.86 (d, J = 8.3 Hz, 2H), 7.67 (d, J = 8.3 Hz, 2H), 7.63 (s, 1H), 4.68 (tt, J = 12.1, 4.1 Hz, 1H), 4.43 (s, 2H), 4.24 (d, J = 11.2 Hz, 1H), 3.74 (ddd, J = 15.3, 11.0, 4.2 Hz, 1H), 3.64-3.59 (m, 1H), 3.56- 3.45 (m, 3H), 3.16-3.09 (m, 2H), 2.19-2.12 (m, 4H), 2.11-2.04 (m, 2H), 1.59-1.53 (m, 2H), 1.48 (s, 9H). MS (ESI) m/z calcd for C₃₆H₄₁F₆N₆O₃⁺: 719.30 [M + H]⁺; found m/z 719.30. |
| 27 | 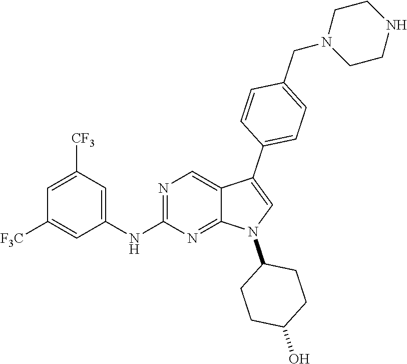 | ++++ | ++ | ++ | ¹H NMR (850 MHz, DMSO-d₆) δ 10.32 (s, 1H), 9.17 (s, 1H), 8.60 (s, 2H), 8.01 (s, 1H), 7.86 (d, J = 8.3 Hz, 2H), 7.65 (d, J = 8.3 Hz, 2H), 7.55 (s, 1H), 4.56-4.51 (m, 1H), 4.41 (s, 2H), 3.68-3.65 (m, 9H), 2.03-1.95 (m, 6H), 1.41-1.34 (m, 2H). MS (ESI) m/z calcd for C₃₁H₃₃F₆N₆O⁺: 619.25 [M + H]⁺; found m/z 619.30. |
| 28 | 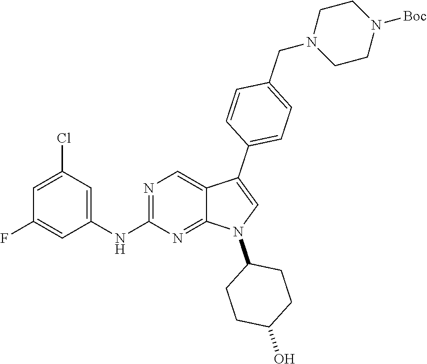 | ++++ | ++ | ++ | ¹H NMR (850 MHz, DMSO-d₆) δ 9.94 (s, 1H), 9.08 (s, 1H), 7.92 (s, 1H), 7.80 (s, 1H), 7.72-7.69 (m, 1H), 7.68 (d, J = 8.2 Hz, 2H), 7.34 (d, J = 8.2 Hz, 2H), 6.90-6.85 (m, 1H), 4.91 (d, J = 4.3 Hz, 1H), 4.51-4.44 (m, 1H), 3.67- 3.51 (m, 6H), 2.31 (t, J = 5.2 Hz, 4H), 2.06-1.96 (m, 6H), 1.44-1.38 (m, 1H), 1.37 (s, 9H). MS (ESI) m/z calcd for C₃₄H₄₁FClN₆O₃⁺: 635.28 [M + H]⁺; found m/z 635.30. |
| 29 | 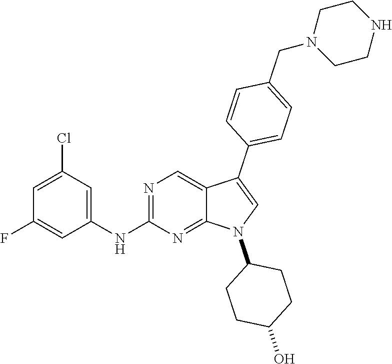 | ++++ | ++++ | +++ | ¹H NMR (850 MHz, DMSO-d₆) δ 10.01 (s, 1H), 9.12 (s, 1H), 7.94 (s, 1H), 7.79-7.69 (m, 4H), 7.37 (s, 1H), 6.90 (d, J = 8.3 Hz, 1H), 4.53-4.48 (m, 1H), 3.64-3.53 (m, 2H), 3.08 (s, 2H), 2.59 (d, J = 19.7 Hz, 3H), 2.08- 1.94 (m, 8H), 1.45-1.38 (m, 2H), 1.26-1.18 (m, 2H). MS (ESI) m/z calcd for C₂₉H₃₄CFlN₆O⁺: 535.23 [M + H]⁺; found m/z 535.20. |
| 30 | 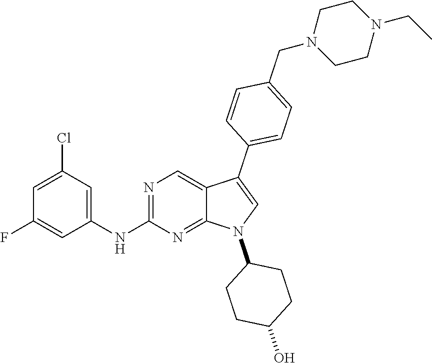 | ++++ | ++++ | +++ | ¹H NMR (850 MHz, DMSO-d₆) δ 10.08 (s, 1H), 9.19 (s, 1H), 8.00 (d, J = 3.8 Hz, 1H), 7.94 (s, 1H), 7.87 (dd, J = 8.3, 1.8 Hz, 2H), 7.74 (dt, J = 12.1, 2.2 Hz, 1H), 7.70 (d, J = 8.3 Hz, 1H), 7.65 (d, J = 8.3 Hz, 1H), 6.92 (dt, J = 8.4, 2.1 Hz, 1H), 4.54-4.49 (m, 1H), 4.39 (d, J = 53.8 Hz, 1H), 3.63-3.58 (m, 4H), 3.51-3.49 (m, 8H), 2.08 1.97 (m, 7H), 1.46-1.38 (m, 4H). MS (ESI) m/z calcd for C₃₁H₃₇CFlN₆O⁺: 563.26 [M + H]⁺; found m/z 563.30. |
| 31 | 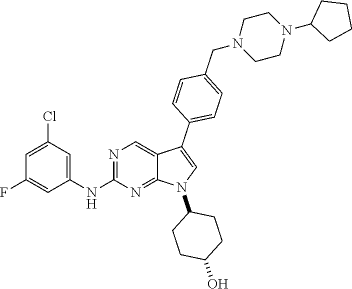 | ++++ | ++++ | +++ | ¹H NMR (850 MHz, DMSO-d₆) δ 10.02 (s, 1H), 9.17 (s, 1H), 7.97 (s, 1H), 7.94 (s, 1H), 7.86 (d, J = 7.9 Hz, 2H), 7.74 (dt, J = 12.0, 2.2 Hz, 1H), 7.63 (d, J = 7.8 Hz, 2H), 6.91 (dt, J = 8.4, 2.2 Hz, 1H), 4.54-4.48 (m, 2H), 4.38-4.32 (m, 2H), 3.79-3.48 (m, 6H), 2.73-2.72 (m, 1H), 2.08-1.98 (m, 12H), 1.76-1.69 (m, 2H), 1.58- 1.52 (m, 2H), 1.46-1.38 (m, 2H). MS (ESI) m/z calcd for C₃₄H₄₁ClFN₆O⁺: 603.29 [M + H]⁺; found m/z 603.30. |
| 32 | 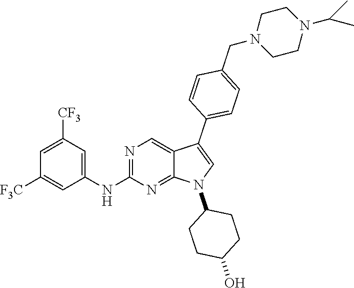 | ++++ | ++ | ++ | ¹H NMR (850 MHz, DMSO-d₆) δ 10.39 (s, 1H), 9.22 (s, 1H), 8.64 (s, 2H), 8.06 (s, 1H), 7.89 (d, J = 7.9 Hz, 2H), 7.71 (d, J = 7.7 Hz, 2H), 7.56 (s, 1H), 4.59-4.54 (m, 1H), 4.44-4.40 (m, 1H), 3.69-3.55 (m, 11H), 2.06- 1.95 (m, 6H), 1.42-1.36 (m, 2H), 1.30-1.25 (m, 6H). MS (ESI) m/z calcd for C₃₄H₃₉F₆N₆O⁺: 661.30 [M + H]⁺; found m/z 661.30. |
| 33 | 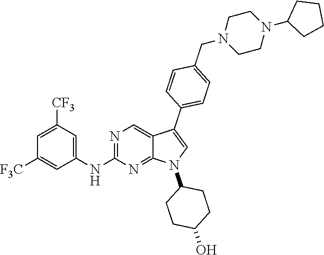 | ++++ | ++ | ++ | ¹H NMR (850 MHz, DMSO-d₆) δ 10.37 (s, 1H), 9.22 (s, 1H), 8.64 (s, 2H), 8.05 (s, 1H), 7.88 (d, J = 7.8 Hz, 2H), 7.67 (s, 2H), 7.56 (s, 1H), 4.59- 4.53 (m, 1H), 4.43-4.37 (m, 1H), 3.61-3.54 (m, 7H), 2.33-2.24 (m, 4H), 2.07-1.96 (m, 10H), 1.94-1.91 (m, 2H), 1.82-1.76 (m, 2H), 1.45- 1.37 (m, 2H). MS (ESI) m/z calcd for (C₃₆H₄₁FN₆O⁺): 687.32 [M + H]⁺; found m/z 687.20. |
| 34 | 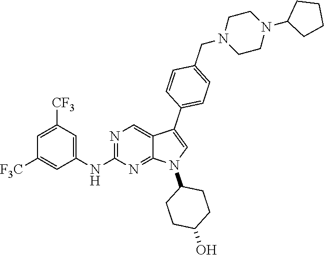 | ++++ | ++++ | +++ | ¹H NMR (850 MHz, DMSO-d₆) δ 9.97 (s, 1H), 9.13 (s, 1H), 7.92 (s, 2H), 7.82 (d, J = 8.0 Hz, 2H), 7.70 (dt, J = 12.0, 2.2 Hz, 1H), 7.56 (d, J = 7.9 Hz, 2H), 6.90 (dt, J = 8.4, 2.2 Hz, 1H), 4.49 (t, J = 11.7 Hz, 1H), 4.24 (s, 1H), 3.84- 3.61 (m, 11H), 2.31-2.25 (m, 2H), 2.19-2.14 (m, 2H), 2.05-1.97 (m, 8H), 1.94-1.89 (m, 2H), 1.81-1.75 (m, 2H), 1.41 (dt, J = 16.1, 10.8 Hz, 2H). MS (ESI) m/z calcd for C₃₆H₄₃ClFN₆O⁺: 629.30 [M + H]⁺; found m/z 629.20. |
| 35 | 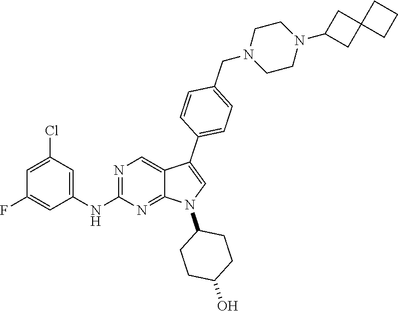 | ++++ | ++ | ++ | ¹H NMR (850 MHz, DMSO-d₆) δ 10.41 (s, 1H), 9.23 (s, 1H), 8.64 (s, 2H), 8.07-8.05 (m, 1H), 7.89 (d, J = 7.6 Hz, 2H), 7.71 (d, J = 8.3 Hz, 2H), 7.57 (s, 1H), 4.59-4.54 (m, 1H), 4.43 (s, 1H), 3.66-3.43 (m, 13H), 2.04- 1.96 (m, 8H), 1.82-1.75 (m, 2H), 1.74-1.69 (m, 2H), 1.54 (s, 2H), 1.44- 1.36 (m, 2H). MS (ESI) m/z calcd for C₃₈H₄₃F₆N₆O₃⁺: 713.33 [M + H]⁺; found m/z 713.30. |
| 36 | 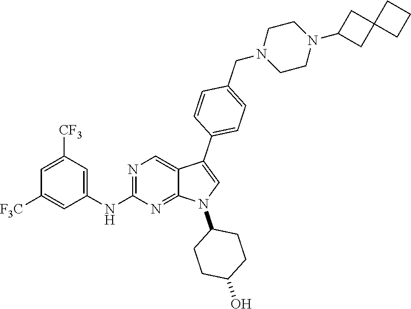 | +++ | + | + | ¹H NMR (400 MHz, DMSO-d₆) δ 10.49 (s, 1H), 9.12 (d, J = 4.7 Hz, 1H), 8.61 (s, 2H), 7.87 (d, J = 2.3 Hz, 2H), 7.70 (t, J = 8.4 Hz, 2H), 7.57 (s, 2H), 7.09 (dd, J = 20.1, 8.9 Hz, 2H), 4.64- 4.46 (m, 1H), 3.62-3.53 (m, 1H), 3.47 (d, J = 10.9 Hz, 1H), 3.29 (d, J = 9.7 Hz, 1H), 3.23-3.04 (m, 4H), 2.75 (dd, J = 11.1, 4.8 Hz, 3H), 2.29-2.12 (m, 2H), 2.10-1.89 (m, 9H), 1.38 (t, J = 7.7 Hz, 2H). MS (ESI) m/z calcd for C₃₂H₃₄F₆N₅O₂: 634.25 [M + H]⁺; found 634.20. |
| 37 | 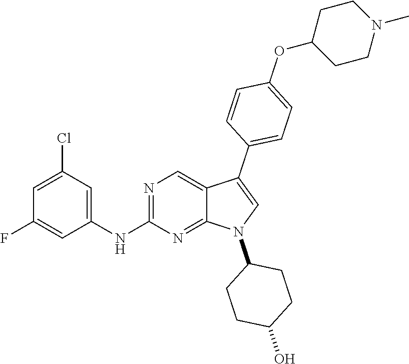 | ++++ | ++++ | +++ | ¹H NMR (400 MHz, DMSO-d₆) δ 10.44 (s, 1H), 9.22 (s, 1H), 8.63 (s, 2H), 8.09 (s, 1H), 7.89 (d, J = 8.5 Hz, 2H), 7.57 (s, 1H), 7.53 (d, J = 8.5 Hz, 2H), 4.62-4.50 (m, 1H), 3.60-3.53 (m, 6H), 3.46-3.38 (m, 3H), 3.16- 3.04 (m, 2H), 2.78 (s, 3H), 2.01 (d, J = 6.7 Hz, 6H), 1.49-1.32 (m, 2H). MS (ESI) m/z calcd for C₃₀H₃₄ClFN₅O₂: 550.23 [M + H]⁺; found 550.20. |
| 38 | 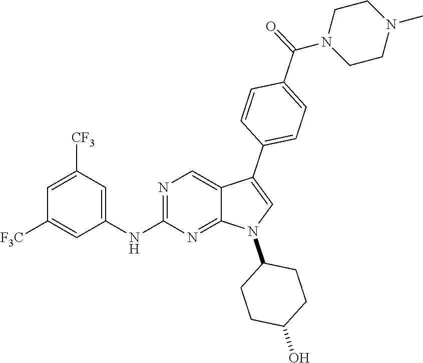 | ++++ | ++ | − | ¹H NMR (400 MHz, DMSO-d₆) δ 10.08 (s, 1H), 9.19 (s, 1H), 8.02 (s, 1H), 7.94 (s, 1H), 7.87 (d, J = 8.5 Hz, 2H), 7.74 (dt, J = 12.2, 2.2 Hz, 1H), 7.52 (d, J = 8.4 Hz, 2H), 6.94-6.89 (m, 1H), 4.57-4.45 (m, 1H), 3.67 3.56 (m, 2H), 3.44-3.31 (m, 6H), 3.15-3.04 (m, 3H), 2.78 (s, 3H), 2.10- 1.98 (m, 7H), 1.53-1.35 (m, 2H). MS (ESI) m/z calcd for C₃₂H₃₃F₆N₆O₂: 647.25 [M + H]⁺; found 647.20. |
| 64 | 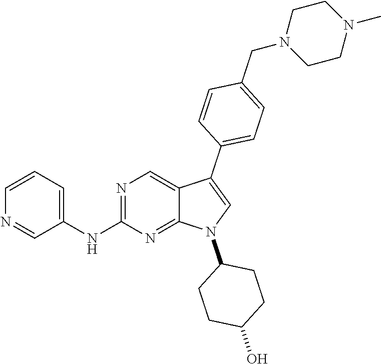 | ++++ | ++++ | ++++ | ¹H NMR (400 MHz, DMSO-d₆) δ 10.61 (s, 1H), 9.55 (d, J = 2.5 Hz, 1H), 9.23 (s, 1H), 8.65 (d, J = 2.4 Hz, 1H), 8.45 (d, J = 5.4 Hz, 1H), 8.07 (s, 1H), 7.97 (dd, J = 8.8, 5.4 Hz, 1H), 7.85 (d, J = 8.1 Hz, 2H), 7.69 (d, J = 8.1 Hz, 2H), 4.64-4.52 (m, 1H), 4.39 (s, 2H), 3.68-3.56 (m, 6H), 2.79 (s, 4H), 2.03- 1.89 (m, 7H), 1.54-1.38 (m, 2H). MS (ESI) m/z calcd for C₂₉H₃₆N₇O: 498.29 [M + H]⁺; found 498.30. |
| 66 | 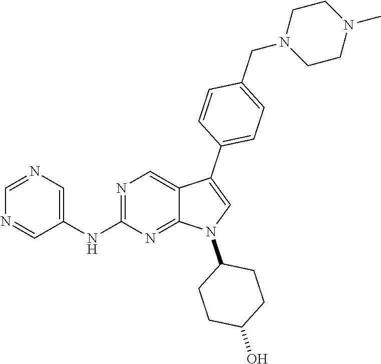 | ++++ | ++++ | ++++ | ¹H NMR (400 MHz, DMSO-d₆) δ 10.06 (s, 1H), 9.27 (s, 1H), 9.17 (s, 1H), 8.77 (s, 1H), 8.00 (s, 1H), 7.83 (d, J = 8.1 Hz, 2H), 7.68 (d, J = 8.1 Hz, 2H), 4.53-4.45 (m, 1H), 4.40 (s, 2H), 3.44-3.29 (m, 8H), 2.79 (s, 3H), 2.11- 1.89 (m, 7H), 1.47-1.30 (m, 2H). MS (ESI) m/z calcd for C₂₈H₃₅N₈O: 499.29 [M + H]⁺; found 499.30. |
| 147 | 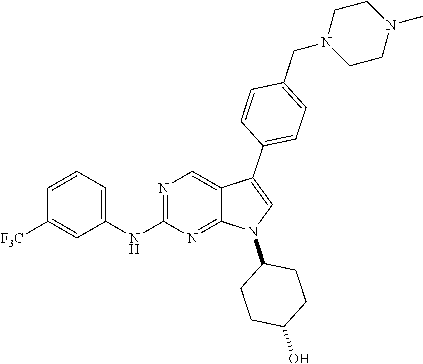 | ++++ | ++++ | | ¹H NMR (500 MHz, DMSO-d₆) δ 10.15 (s, 1H), 9.20 (s, 1H), 8.69 (s, 1H), 8.04 (s, 1H), 7.87 (d, J = 7.9 Hz, 2H), 7.82 (d, J = 2.2 Hz, 1H), 7.71 (d, J = 7.9 Hz, 2H), 7.53 (t, J = 8.0 Hz, 1H), 7.29 (d, J = 7.7 Hz, 1H), 4.60 4.50 (m, 1H), 4.41 (s, 2H), 3.69-3.63 (m, 3H), 3.62-3.58 (m, 4H), 3.40- 3.35 (m, 2H), 2.82 (s, 3H), 2.00 (td, J = 12.3, 5.4 Hz, 6H), 1.46-1.34 (m, 2H). MS (ESI) m/z calcd for C₃₁H₃₆F₃N₆O: 565.28 [M + H]⁺; found 565.30. |
| 148 | 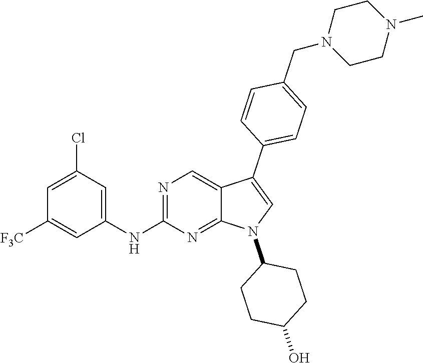 | ++++ | +++ | | ¹H NMR (500 MHz, DMSO-d₆) δ 10.20 (s, 1H), 9.20 (s, 1H), 8.43 (s, 1H), 8.23 (s, 1H), 8.01 (s, 1H), 7.87 (d, J = 7.8 Hz, 2H), 7.66 (d, J = 7.7 Hz, 2H), 7.33 (s, 1H), 4.59-4.49 (m, 1H), 4.33 (s, 2H), 3.67-3.55 (m, 4H), 3.21- 3.14 (m, 5H), 2.81 (s, 3H), 2.07- 1.97 (m, 6H), 1.47-1.35 (m, 2H). MS (ESI) m/z calcd for C₃₁H₃₅ClF₃N₆O: 599.24 [M + H]⁺; found 599.20. |
| 149 | 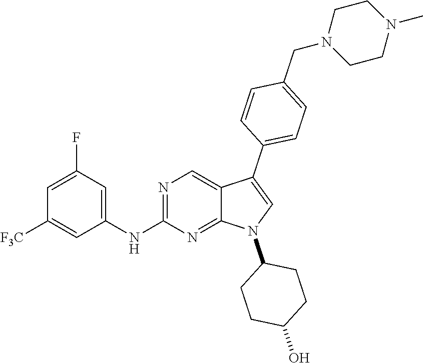 | ++++ | ++++ | | ¹H NMR (500 MHz, DMSO-d₆) δ 10.27 (s, 1H), 9.21 (s, 1H), 8.34 (s, 1H), 8.04 (s, 1H), 7.97-7.90 (m, 1H), 7.88 (d, J = 8.0 Hz, 2H), 7.71 (d, J = 8.1 Hz, 2H), 7.18-7.13 (m, 1H), 4.59- 4.49 (m, 1H), 4.42 (s, 2H), 3.69- 3.63 (m, 4H), 3.51-3.47 (m, 2H), 3.42-3.36 (m, 2H), 2.82 (s, 3H), 2.07- 1.97 (m, 6H), 1.46-1.34 (m, 2H). MS (ESI) m/z calcd for C₃₁H₃₅F₄N₆O: 583.27 [M + H]⁺; found 583.30. |
| 151 | 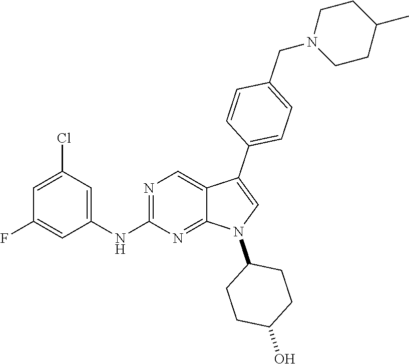 | ++++ | ++++ | | ¹H NMR (500 MHz, CD₃OD) δ 9.06 (s, 1H), 8.00 (s, 1H), 7.84 (d, J = 8.2 Hz, 2H), 7.78 (s, 1H), 7.65 (d, J = 8.2 Hz, 2H), 7.52 (dt, J = 10.8, 2.1 Hz, 1H), 6.99 (d, J = 8.4 Hz, 1H), 4.66- 4.55 (m, 1H), 4.35 (s, 2H), 3.82-3.72 (m, 1H), 3.51 (d, J = 12.3 Hz, 2H), 3.10-2.98 (m, 2H), 2.17 (q, J = 6.7 Hz, 6H), 1.98-1.88 (m, 2H), 1.80- 1.67 (m, 1H), 1.65-1.54 (m, 2H), 1.51-1.42 (m, 2H), 1.41-1.26 (m, 2H), 1.02 (d, J = 6.5 Hz, 3H).C₃₁H₃₆ClFN₅O⁺: 548.25 [M + H]⁺; found m/z 548.30. |
| 174 | 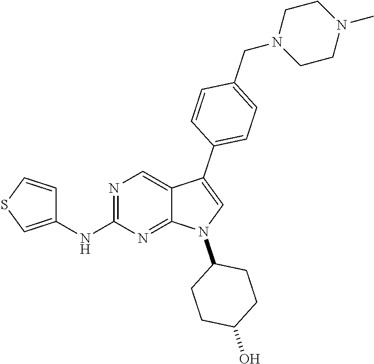 | ++++ | ++++ | | ¹H NMR (400 MHz, DMSO-d₆) δ 10.10 (s, 1H), 9.13 (s, 1H), 7.96 (s, 1H), 7.84 (d, J = 8.0 Hz, 2H), 7.71 (dd, J = 3.2, 1.3 Hz, 1H), 7.67 (d, J = 8.0 Hz, 2H), 7.53-7.44 (m, 2H), 7.24 (dd, J = 5.2, 1.3 Hz, 1H), 4.58-4.52 (m, 1H), 4.42-4.30 (m, 2H), 3.68-3.55 (m, 8H), 2.82 (s, 3H), 2.01 (d, J = 13.2 Hz, 7H), 1.50-1.42 (m, 2H). MS (ESI) m/z calcd for C₂₈H₃₅N₆OS: 503.25 [M + H]⁺; found 503.30. |
| 175 | 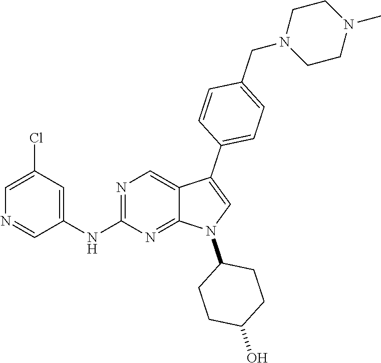 | ++++ | ++++ | | ¹H NMR (400 MHz, DMSO-d₆) δ 10.17 (s, 1H), 9.21 (s, 1H), 8.86 (d, J = 2.2 Hz, 1H), 8.73 (t, J = 2.2 Hz, 1H), 8.21 (d, J = 2.2 Hz, 1H), 8.01 (s, 1H), 7.87 (d, J = 8.0 Hz, 2H), 7.69 (d, J = 8.0 Hz, 2H), 4.51 (td, J = 10.4, 4.8 Hz, 1H), 4.41 (s, 2H), 3.69-3.55 (m, 8H), 2.83 (s, 3H), 2.09-1.96 (m, 6H), 1.51- 1.36 (m, 2H). MS (ESI) m/z calcd for C₂₉H₃₅ClN₇O: 532.25 [M + H]⁺; found 532.30. |

### Example 2—Syntheses Cis-4-(2-((3,5-bis(trifluoromethyl)phenyl)amino)-5-(4-((4-methylpiperazin-1-yl)methyl)phenyl)-7H-pyrrolo[2,3-d]pyrimidin-7-yl)cyclohexan-1-ol

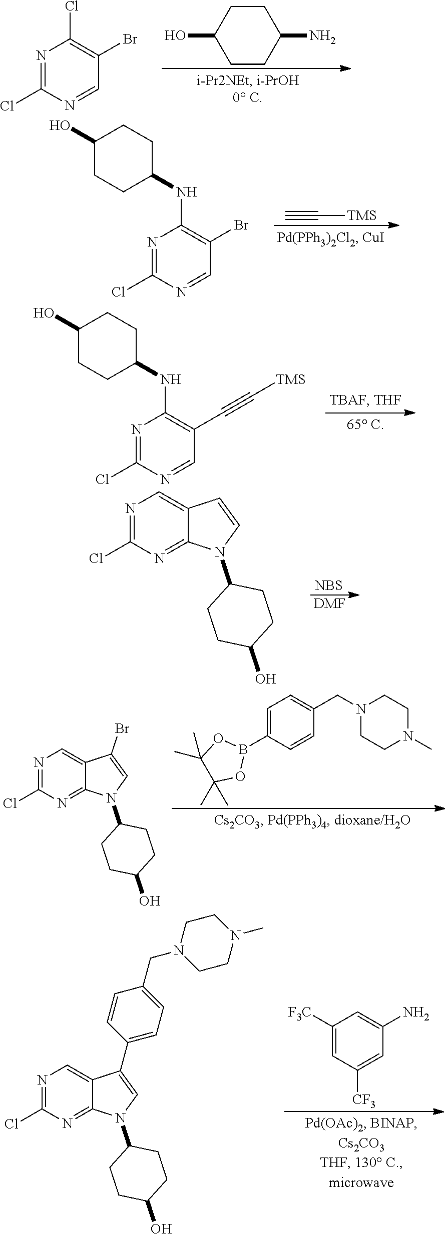

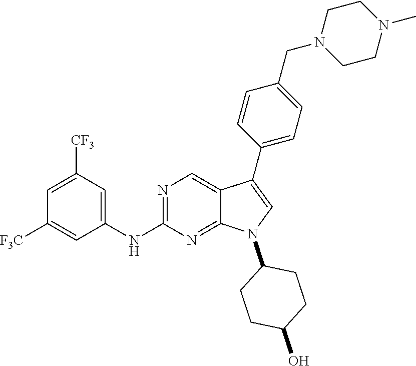

To a solution of 2,4-dichloro-5-bromopyrimidine (10.0 g, 43.9 mmol) and N,N-diisopropylethylamine (8.51 g, 65.8 mmol) in anhydrous isopropyl alcohol (125 mL) was slowly added cis-4-aminocyclohexanol (6.99 g, 46.1 mmol) in anhydrous isopropyl alcohol (75 mL) at 0° C. The resulting reaction mixture was stirred at 0° C. for 4 h, then warmed to room temperature and stirred for another 4 h. Then solvent was removed under reduced pressure. The residue was dissolved in EtOAc (150 mL), washed with water (50 mL) and brine, dried (Na₂SO₄), and concentrated. The title compound cis-(1s,4s)-4-((5-bromo-2-chloropyrimidin-4-yl)amino)cyclohexan-1-ol was obtained as a white solid (9.8 g) after recrystallization from a mixture of EtOAc and Hexane (1:5). The yield is 73.0%, ¹H NMR (400 MHz, CD₃OD) δ 8.12 (s, 1H), 4.07 (tt, J=9.9, 4.1 Hz, 1H), 3.96-3.90 (m, 1H), 1.88-1.76 (m, 4H), 1.76-1.64 (m, 4H). MS (ESI) m/z calcd for C₁₀H₁₄BrClN₃O: 305.99 [M+H]⁺; found 308.0. LC-MS: >96% purity.

To a solution of (1s,4s)-4-((5-bromo-2-chloropyrimidin-4-yl)amino)cyclohexan-1-ol (5.0 g, 16.3 mmol) in anhydrous toluene (100 mL) was added ethynyltrimethylsilane (1.6 g, 16.3 mmol), copper (I) iodide (93.2 mg, 0.49 mmol), Pd(PPh₃)₂Cl₂ (343 mg, 0.49 mmol) and triethylamine (8.25 g, 81.5 mmol) at room temperature. After thoroughly degassing, the resulting mixture was heated under nitrogen atmosphere at 60° C. for 150 min, then quenched with water, and partitioned with EtOAc. The organic layer was dried (Na₂SO₄) and concentrated. The residue was purified by an ISCO silica gel column (EtOAc/hexane 0-100%) to afford the title compound (4.5 g, 85%) as a white solid. ¹H NMR (400 MHz, CDCl₃) δ 8.07 (s, 1H), 5.66 (d, J=8.0 Hz, 1H), 4.17-4.07 (m, 1H), 4.01-3.93 (m, 1H), 1.85-1.68 (m, 7H), 1.65-1.60 (m, 2H), 0.29-0.26 (m, 9H). MS (ESI) m/z calcd for C₁₅H₂₃ClN₃OSi: 324.12 [M+H]⁺; found 324.10. LC-MS: >96% purity.

To a solution of (1s,4s)-4-((2-chloro-5-((trimethylsilyl) ethynyl)pyrimidin-4 yl)amino)cyclohexan-1-ol (1.70 mg, 5.25 mmol) in anhydrous THF (30 mL) was added a 1.0 M THF solution of tetrabutylammonium (2.55 g, 10.5 mmol) at room temperature. The resulting mixture was heated at 65° C. for 5 h, quenched with water (10 mL), and extracted with EtOAc (3×30 mL). The combined organic layers were dried (Na₂SO₄) and concentrated. The residue was purified by an ISCO silica gel column to afford the title compound (1s,4s)-4-(2-chloro-7H-pyrrolo[2,3-d]pyrimidin-7-yl)cyclohexan-1-ol (0.77 g, 58% yield) as a white solid. ¹H NMR (400 MHz, CDCl₃) δ 8.77 (s, 1H), 7.37 (d, J=3.7 Hz, 1H), 6.56 (d, J=3.7 Hz, 1H), 4.77 (tt, J=12.2, 3.7 Hz, 1H), 4.19 (s, 1H), 2.18 (qd, J=13.4, 5.0 Hz, 2H), 1.98 (d, J=16.2 Hz, 2H), 1.82 (dd, J=24.8, 10.8 Hz, 4H). MS (ESI) m/z calcd for C₁₂H₁₅ClN₃O:252.08 [M+H]⁺; found 252.10. LC-MS: >96% purity.

To a solution of (1s,4s)-4-(2-chloro-7H-pyrrolo[2,3-d]pyrimidin-7-yl)cyclohexan-1-ol (770.0 mg, 3.06 mmol) in anhydrous THF (15 mL) was added NBS (544.0 mg, 3.06 mmol) in several portions at room temperature. The resulting mixture was stirred at room temperature until the reaction was complete (based on TLC), quenched with water (10 mL), and extracted with CH₂Cl₂ (3×20 mL). The combined organic layers were dried (Na₂SO₄) and concentrated. The residue was purified by an ISCO silica gel column to afford the title compound (1s,4s)-4-(5-bromo-2-chloro-7H-pyrrolo[2,3-d]pyrimidin-7-yl)cyclohexan-1-ol (550.0 mg, 54.4% yield) as a white solid. ¹H NMR (400 MHz, CDCl₃) δ 8.73 (s, 1H), 7.39 (s, 1H), 4.78 (tt, J=12.3, 3.9 Hz, 1H), 4.19 (d, J=2.3 Hz, 1H), 2.22-2.09 (m, 2H), 1.98 (d, J=15.0 Hz, 2H), 1.90-1.74 (m, 4H). MS (ESI) m/z calcd for C₁₂H₁₄BrClN₃O: 328.99 [M+H]⁺; found 332.0. LC-MS: >96% purity.

To a solution of (1s,4s)-4-(5-bromo-2-chloro-7H-pyrrolo[2,3-d]pyrimidin-7-yl)cyclohexan-1-ol (250 mg, 0.76 mmol) in 1,4-dioxane (10.0 mL) and water (1.0 mL) were added 1-methyl-4-(4-(4,4,5,5-tetramethyl-1,3,2-dioxaborolan-2-yl)-benzyl) piperazine (251 mg, 0.79 mmol), cesium carbonate (370 mg, 1.13 mmol), and Pd(PPh₃)₄ (35 mg, 0.032 mmol). The reaction mixture was heated under nitrogen atmosphere at 90° C. for 12 h. Then the reaction mixture was cooled to room temperature, quenched with H₂O, extracted with EtOAc (3×15 mL). The combined organic layer was washed with brine, dried (MgSO₄), and concentrated under reduced pressure. The residue was purified by an ISCO reversed column (methanol/water+0.1% HCl, 10-100%) to afford a slight yellow solid, which was triturated with methanol. The solid was filtered to afford the desired product (1s,4s)-4-(2-chloro-5-(4-((4-methylpiperazin-1-yl)methyl)phenyl)-7H-pyrrolo[2,3-d]pyrimidin-7-yl)cyclohexan-1-ol (290 mg, 87.2%) as a yellow solid. ¹H NMR (400 MHz, CDCl₃) δ 9.01 (s, 1H), 7.52 (d, J=8.2 Hz, 2H), 7.39 (d, J=8.2 Hz, 2H), 7.24 (s, 1H), 4.80 (tt, J=12.0, 3.6 Hz, 1H), 4.20-4.16 (m, 1H), 3.56 (s, 2H), 2.60 (s, 3H), 2.29-2.16 (m, 4H), 2.01-1.95 (m, 3H), 1.92-1.75 (m, 7H), 1.33-1.30 (m, 2H). MS (ESI) m/z calcd for C₂₄H₃₁ClN₅O: 440.21 [M+H]⁺; found 440.20. LC-MS: >96% purity.

To a solution of tert-butyl 4-(2-amino-4-((cis-4-hydroxycyclohexyl)amino)pyrimidin-5-yl)-5,6-dihydropyridine-1 (2H)-carboxylate (60.0 mg, 0.14 mmol) and 3,5-dichloroaniline (94.0 mg, 0.41 mmol) in dry THF (10 mL) were added palladium (II) acetate (9.20 mg, 41 μmol), BINAP (25.0 mg, 41 μmol), and Cs₂CO₃ (110 mg, 0.34 mmol). The reaction mixture was heated under nitrogen atmosphere and microwave radiation at 130° C. for 0.5 h, then quenched by water. The mixture was extracted with EtOAc (3×15 mL). The combined organic layer was washed with water and brine, dried (Na₂SO₄), and concentrated. The residue was purified by an ISCO reversed column (methanol/water+0.1% HCl, 10-100%) to afford crude product which was recrystallized from methanol to afford the title compound (25 mg, 0.14 mmol, 29%) as a yellow solid. ¹H NMR (850 MHz, CD₃OD) δ 9.13 (s, 1H), 8.45 (s, 2H), 8.11 (s, 1H), 7.87 (d, J=8.2 Hz, 2H), 7.77-7.73 (m, 3H), 4.73 (tt, J=12.3, 4.0 Hz, 1H), 4.50 (s, 2H), 4.12 (p, J=2.9 Hz, 1H), 3.69 (m, 9H), 3.02 (s, 3H), 2.31 (m, 2H), 2.06-2.01 (m, 4H), 1.91 (dd, J=12.7, 4.3 Hz, 2H), 1.78-1.73 (m, 2H). MS (ESI) m/z calcd for C₃₂H₃₅F₆N₆O⁺: 633.27 [M+H]⁺; found m/z 633.30.

Additional compounds can be prepared following the synthesis procedure described in Example 2 using the corresponding reagents.

Table 3 describes compounds prepared following procedures described in Example 2 using appropriate reagents. Biological assays were performed as described in Example 5 (Note: Mer IC₅₀: ++++ means <10 nM; +++ means between 10-100 nM, ++ means between 100 nM-1 μM; + means between 1-30 M; − means inactive).

TABLE 3

| | | | | | Physical Data |
|---|---|---|---|---|---|
| Cpd. | | TYRO3 | MERTK | AXL | MS m/z (M + 1) |
| No. | Structure | IC₅₀ | IC₅₀ | IC₅₀ | or/and ¹H NMR |
| 39 | 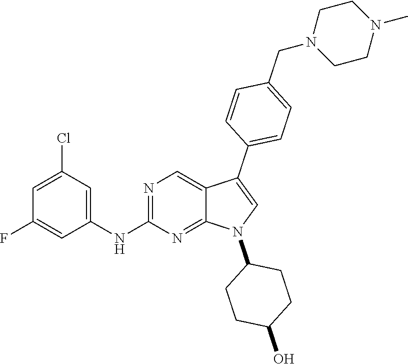 | ++++ | ++++ | +++ | ¹H NMR (850 MHz, CD₃OD) δ 9.07 (s, 1H), 8.10 (s, 1H), 7.86 (d, J = 8.1 Hz, 2H), 7.75 (d, J = 7.8 Hz, 2H), 7.72 (s, 1H), 7.51 (dt, J = 10.5, 2.1 Hz, 1H), 7.04 (dt, J = 8.4, 2.1 Hz, 1H), 4.70 (tt, J = 12.2, 3.9 Hz, 1H), 4.48 (s, 2H), 4.12 (p, J = 2.9 Hz, 1H), 3.68 (s, 10H), 3.02 (s, 3H), 2.33 (qd, J = 12.7, 3.6 Hz, 2H), 2.04 (d, J = 15.1 Hz, 2H), 1.95 (d, J = 10.4 Hz, 2H), 1.81 (t, J = 13.1 Hz, 2H). MS (ESI) m/z calcd for C₃₀H₃₅ClFN₆O⁺: 549.25 [M + H]⁺; found m/z 549.20. |
| 40 | 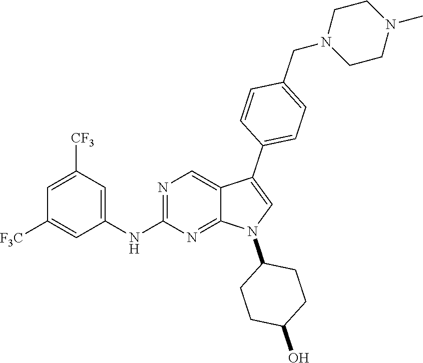 | ++++ | + | ++ | ¹H NMR (850 MHz, CD₃OD) δ 9.13 (s, 1H), 8.45 (s, 2H), 8.11 (s, 1H), 7.87 (d, J = 8.2 Hz, 2H), 7.77-7.73 (m, 3H). 4.73 (tt, J = 12.3, 4.0 Hz, 1H), 4.50 (s, 2H), 4.12 (p, J = 2.9 Hz, 1H), 3.69 (m, 9H), 3.02 (s, 3H), 2.31 (m, 2H), 2.06-2.01 (m, 4H), 1.91 (dd, J = 12.7, 4.3 Hz, 2H), 1.78-1.73 (m, 2H). MS (ESI) m/z calcd for C₃₂H₃₅F₆N₆O⁺: 633.27 [M + H]⁺; found m/z 633.30. |

### Example 3—Syntheses trans-4-(2-((3-Chloro-5-fluorophenyl)amino)-5-(4-((4-methylpiperazin-1-yl)methyl)phenyl)imidazo[5,1-f][1,2,4]triazin-7-yl)cyclohexan-1-ol

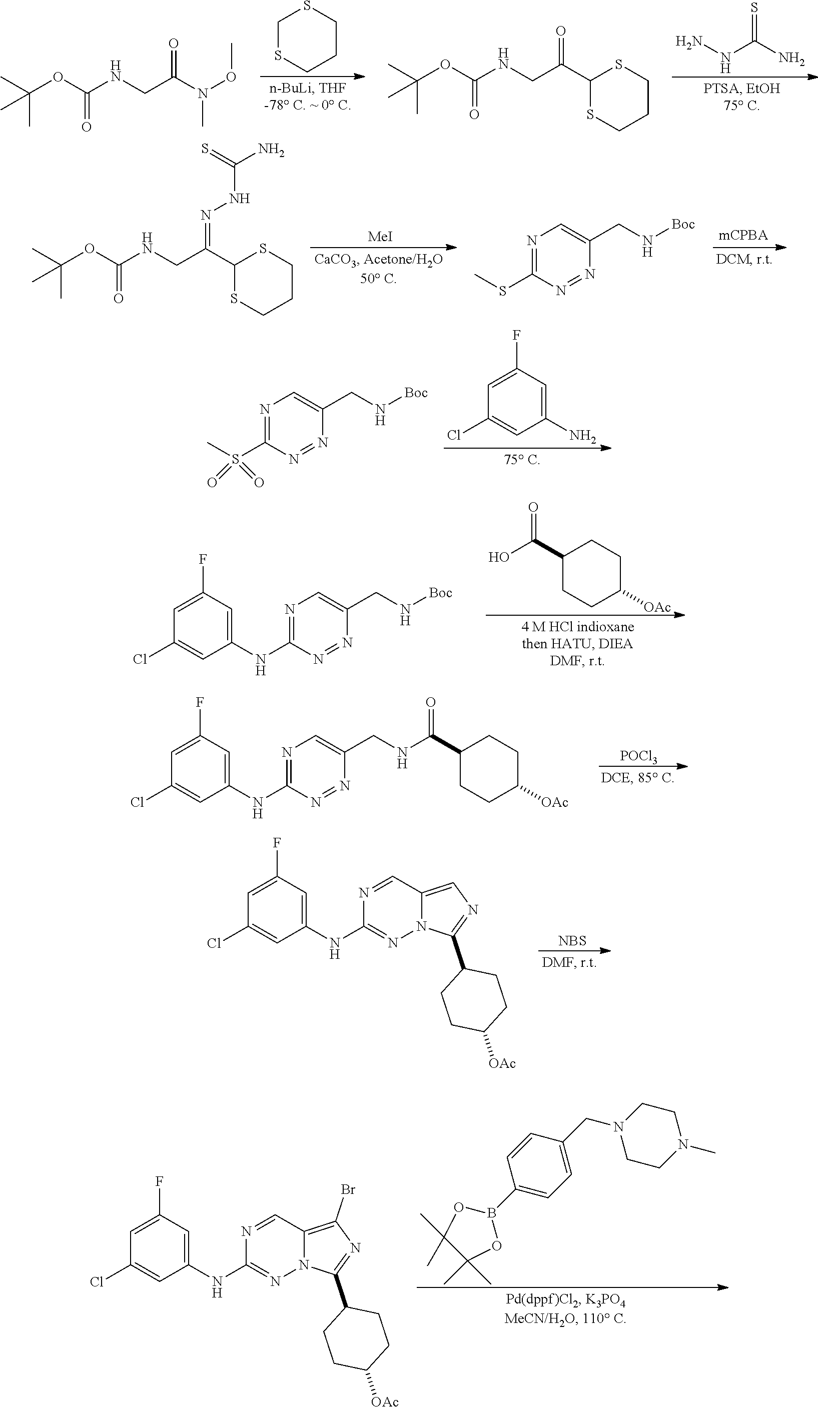

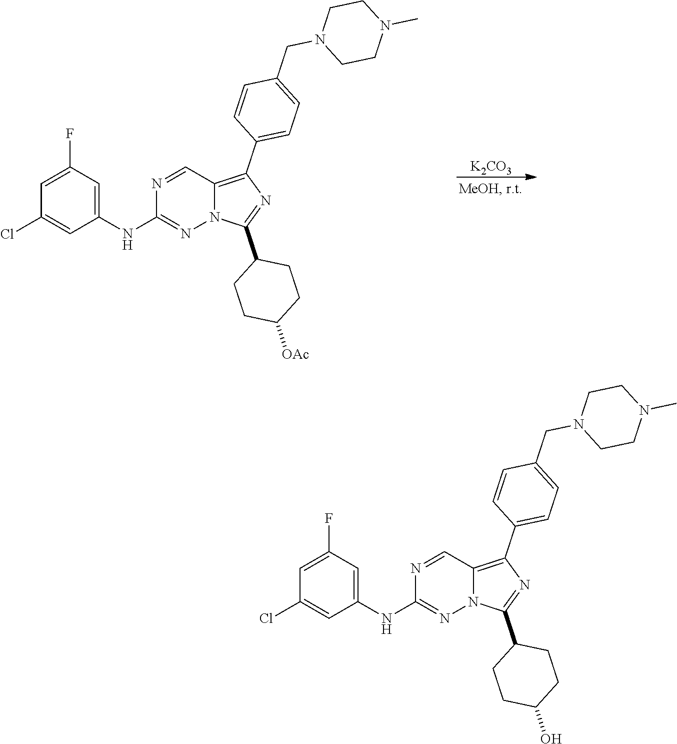

To a solution of 1,3-dithiane (6.37 g, 53.0 mmol) in tetrahydrofuran (80 mL) was added a 2.5 M solution of n-butyllithium in hexane (50.5 mmol, 20.2 mL) at 0° C. slowly. After stirring at 0° C. for 30 min, the reaction mixture was cooled to −70° C., and was added a solution of tert-butyl {2-[methoxy(methyl)amino]-2-oxoethyl}carbamate (5.51 g, 25.2 mmol) in tetrahydrofuran (20 mL) slowly. The resulting mixture was warmed to about 0° C. slowly, stirred at 0° C. for 4 h, quenched with a saturated aqueous NH₄Cl solution, and extracted with ethyl acetate (3×). The combined organic phase was washed with brine, and dried (Na₂SO₄). Solvents were removed under reduced pressure and the resulting residue was purified by an ISCO silica gel column to yield tert-butyl(2-(1,3-dithian-2-yl)-2-oxoethyl) carbamate as a white solid (4.55 g, 65%). ¹H NMR (400 MHz, Chloroform-d) δ 5.20 (s, 1H), 4.21 (s, 2H), 3.33-3.10 (m, 2H), 2.64-2.48 (m, 2H), 2.16-1.88 (m, 2H), 1.41 (s, 9H).

To a solution of tert-butyl(2-(1,3-dithian-2-yl)-2-oxoethyl) carbamate (6.99 g, 25.2 mmol) and hydrazinecarbothioamide (2.30 g, 25.2 mmol) in ethanol (50 mL) was added p-toluenesulfonic acid (217 mg, 1.26 mmol). The mixture was heated at 75° C. for 12 h. After that, majority of the ethanol was removed under reduced pressure before MTBE (30 mL) was added. White solids participated and then filtered. Solids were collected and washed with MTBE to afford tert-butyl-(2-(2-carbamoylhydrazono)-2-(1,3-dithian-2-yl)ethyl) carbamate as a white solid (7.33 g, 83%).

To a suspension of tert-butyl-(2-(2-carbamothioylhydrazineylidene)-2-(1,3-dithian-2-yl)ethyl) carbamate (6.80 g, 19.4 mmol) and calcium carbonate (5.83 g, 58.2 mmol) in a mixture of acetone/water (160 mL/20 mL) was added a solution of methyl iodide (12.1 mL, 194 mmol) in acetone (20 mL). The mixture was stirred at 50° C. for 12 h. Then, solvents were removed under reduced pressure and the residue was purified by an ISCO silica gel column to afford tert-butyl ((3-(methylthio)-1,2,4-triazin-6-yl)methyl) carbamate as a white solid (1.24 g, 25%). ¹H NMR (400 MHz, Chloroform-d) δ 8.45 (s, 1H), 5.38 (br, 1H), 4.56 (d, J=6.1 Hz, 2H), 2.66 (s, 3H), 1.45 (s, 9H).

To a solution of tert-butyl ((3-(methylthio)-1,2,4-triazin-6-yl)methyl) carbamate (500 mg, 1.95 mmol) in methylene chloride (30 mL) was added m-chloroperbenzoic acid (1.18 g, 6.83 mmol). The reaction mixture was stirred at rt for 12 h, then, saturated aqueous sodium bicarbonate was added. The suspension was stirred for 10 min and extracted with CH₂Cl₂ (3×). The combined organic phase was washed with brine and dried (Na₂SO₄). Solvents were removed under reduced pressure to yield tert-butyl ((3-(methylsulfonyl)-1,2,4-triazin-6-yl)methyl) carbamate as a white solid (563 mg, quant.). ¹H NMR (400 MHz, Chloroform-d) δ 8.91 (s, 1H), 5.57 (t, J=6.1 Hz, 1H), 4.75 (d, J=6.1 Hz, 2H), 3.46 (s, 3H), 1.44 (s, 9H).

A suspension of tert-butyl ((3-(methylsulfonyl)-1,2,4-triazin-6-yl)methyl) carbamate (500 mg, 1.73 mmol) 3-chloro-5-fluoroaniline was heated at 120° C. for 1.5 h under nitrogen atmosphere. Then, solvent was removed under reduced pressure and the residue was purified by an ISCO silica gel column to afford tert-butyl ((3-((3-chloro-5-fluorophenyl)amino)-1,2,4-triazin-6-yl)methyl) carbamate as a white solid (429 mg, 70%). ¹H NMR (400 MHz, DMSO-d₆) δ 10.51 (s, 1H), 8.48 (s, 1H), 7.73-7.71 (m, 1H), 7.68 (dt, J=11.7, 2.2 Hz, 1H), 7.51 (t, J=5.3 Hz, 1H), 7.02 (dt, J=8.7, 2.1 Hz, 1H), 4.34 (d, J=5.9 Hz, 2H), 1.39 (s, 9H).

A 4.0 M HCl solution in dioxane (3 mL) was added to the solution of tert-butyl ((3-((3-chloro-5-fluorophenyl)amino)-1,2,4-triazin-6-yl)methyl) carbamate (130.0 mg, 367.5 μmol) in MeOH (5 mL). The reaction mixture was stirred at rt for 2 h. Then, solvents were removed under reduced pressure. The residues were redissolved in DMF (5 mL) and was added trans-4-acetoxycyclohexane-1-carboxylic acid (75.27 mg, 404.2 μmol), HATU (168 mg, 441.0 μmol) and DIEA (320 μL, 1.84 mmol). The reaction mixture was stirred at rt for 2h, quenched with a saturated aqueous NH₄Cl solution, and extracted with ethyl acetate (3×). The combined organic phase was washed with brine, dried (Na₂SO₄), and concentrated. The residue was purified by an ISCO silica gel column to afford trans-4-(((3-((3-chloro-5-fluorophenyl)amino)-1,2,4-triazin-6-yl)methyl) carbamoyl)cyclohexyl acetate as a white solid (127 mg, 82%). ¹H NMR (400 MHz, DMSO-d₆) δ 10.51 (s, 1H), 8.55-8.33 (m, 2H), 7.73-7.71 (m, 1H), 7.68 (dt, J=11.7, 2.2 Hz, 1H), 7.02 (dt, J=8.6, 2.1 Hz, 1H), 4.64-4.50 (m, 1H), 4.45 (d, J=5.7 Hz, 2H), 2.33-2.11 (m, 1H), 1.99-1.96 (m, 3H), 1.96-1.78 (m, 4H), 1.52-1.21 (m, 4H).

Phosphorus oxychloride (371 mg, 2.42 mmol) was added to a suspension of trans-4-(((3-((3-chloro-5-fluorophenyl)amino)-1,2,4-triazin-6-yl)methyl) carbamoyl)cyclohexyl acetate (51.0 mg, 121 μmol) in 1,2-dichloroethane (5.0 ml), and the mixture was heated at 85° C. for 12 h under nitrogen atmosphere. After the reaction was cooled to rt, 1 mL of methanol was added and the mixture was stirred for another 10 min. Solvents were removed under reduced pressure and the residue was purified by an ISCO silica gel column to afford trans-4-(2-((3-chloro-5-fluorophenyl)amino)imidazo[5,1-f][1,2,4]triazin-7-yl)cyclohexyl acetate as solids (20 mg, 41%). MS (ESI) m/z calcd for C₁₉H₂₀ClFN₅O₂: 404.13 [M+H]⁺; found 404.20.

To a solution of trans-4-(2-((3-chloro-5-fluorophenyl)amino)imidazo[5,1-f][1,2,4]triazin-7-yl)cyclohexyl acetate (33 mg, 81.7 μmol) in DMF was added N-bromosuccinimide (13.1 mg, 73.5 μmol) at rt. The reaction mixture was stirred at rt for 30 min, quenched with a saturated aqueous NaCl solution, and extracted with ethyl acetate (3×). The combined organic phase was washed with brine, dried (Na₂SO₄), and concentrated. The residue was purified by an ISCO silica gel column to afford trans-4-(5-bromo-2-((3-chloro-5-fluorophenyl)amino)imidazo[5,1-f][1,2,4]triazin-7-yl)cyclohexyl acetate as a yellow solid (36 mg, 92%). MS (ESI) m/z calcd for: C₁₉H₁₈BrClFN₅O₂: 482.03 [M+H]⁺; found 482.10.

To a suspension of potassium phosphate tribasic (25 mg, 0.12 mmol), trans-4-(5-bromo-2-((3-chloro-5-fluorophenyl)amino)imidazo[5,1-f][1,2,4]triazin-7-yl)cyclohexyl acetate (19 mg, 39 μmol) and 1-methyl-4-(4-(4,4,5,5-tetramethyl-1,3,2-dioxaborolan-2-yl)benzyl) piperazine (16 mg, 51 μmol) in MeCN/H₂O (9 mL/1 mL) was added 1,1′-bis(diphenylphosphino) ferrocene-palladium (II) dichloride (5.8 mg, 7.9 μmol). The reaction mixture was degassed and then heated at 110° C. for 1 h under nitrogen atmosphere. After cooling to rt, the mixture was filtered through Celite and the filtration was collected. Solvents were removed under reduced pressure and the residue was purified by an ISCO silica gel column to afford trans-4-(2-((3-chloro-5-fluorophenyl)amino)-5-(4-((4-methylpiperazin-1-yl)methyl)phenyl)imidazo[5,1-f][1,2,4]triazin-7-yl)cyclohexyl acetate as a yellow solid (19 mg, 83%). MS (ESI) m/z calcd for C₃₁H₃₅ClFN₇O₂: 592.25 [M+H]⁺; found 592.30.

To a solution of trans-4-(2-((3-chloro-5-fluorophenyl)amino)-5-(4-((4-methylpiperazin-1-yl)methyl)phenyl)imidazo[5,1-f][1,2,4]triazin-7-yl)cyclohexyl acetate (11 mg, 19 μmol) in methanol (5 mL) was added potassium carbonate (13 mg, 93 μmol). The reaction mixture was stirred at rt for 12 h. Solvents were removed under reduced pressure and the residue was purified by preparative HPLC with MeCN/H₂O (with 0.5% TFA as the modifier) as the eluents to afford trans-4-(2-((3-chloro-5-fluorophenyl)amino)-5-(4-((4-methylpiperazin-1-yl)methyl)phenyl)imidazo[5,1-f][1,2,4]triazin-7-yl)cyclohexan-1-ol TFA salt as a yellow solid (10 mg, 93%). ¹H NMR (400 MHz, DMSO-d₆) δ 10.29 (s, 1H), 9.50 (s, 1H), 8.03 (d, J=8.0 Hz, 2H), 7.87-7.83 (m, 1H), 7.59 (dt, J=11.8, 2.1 Hz, 1H), 7.51 (d, J=8.0 Hz, 2H), 7.01 (dt, J=8.5, 2.1 Hz, 1H), 4.04 (s, 2H), 3.61-3.50 (m, 1H), 3.47-3.17 (m, 8H), 3.16-3.03 (m, 1H), 2.83 (s, 3H), 2.17-1.98 (m, 4H), 1.84-1.69 (m, 2H), 1.49-1.31 (m, 2H). MS (ESI) m/z calcd for C₂₉H₃₃ ClFN₇O: 550.24 [M+H]⁺; found 550.20.

Additional compounds can be prepared following the synthesis procedure described in Example 3 using the corresponding reagents.

Table 4 describes compounds prepared following procedures described in Example 3 using appropriate reagents. Biological assays were performed as described in Example 5 (Note: Mer IC₅₀: ++++ means <10 nM; +++ means between 10-100 nM, ++ means between 100 nM-1 μM; + means between 1-30 μM; − means inactive).

TABLE 4

| | | | | | Physical Data |
|---|---|---|---|---|---|
| Cpd. | | TYRO3 | MERTK | AXL | MS m/z (M + 1) or/and ¹H |
| No. | Structure | IC₅₀ | IC₅₀ | IC₅₀ | NMR |
| 141 | 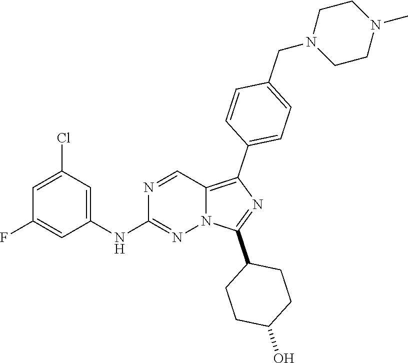 | ++++ | ++++ | ++ | ¹H NMR (400 MHz, DMSO-d₆) δ 10.29 (s, 1H), 9.50 (s, 1H), 8.03 (d, J = 8.0 Hz, 2H), 7.87- 7.83 (m, 1H), 7.59 (dt, J = 11.8, 2.1 Hz, 1H), 7.51 (d, J = 8.0 Hz, 2H), 7.01 (dt, J = 8.5, 2.1 Hz, 1H), 4.04 (s, 2H), 3.61-3.50 (m, 1H), 3.47-3.17 (m, 8H), 3.16-3.03 (m, 1H), 2.83 (s, 3H), 2.17-1.98 (m, 4H), 1.84- 1.69 (m, 2H), 1.49-1.31 (m, 2H). MS (ESI) m/z calcd for C₂₉H₃₃ClFN₇O: 550.24 [M + H]⁺; found 550.20. |
| 142 | 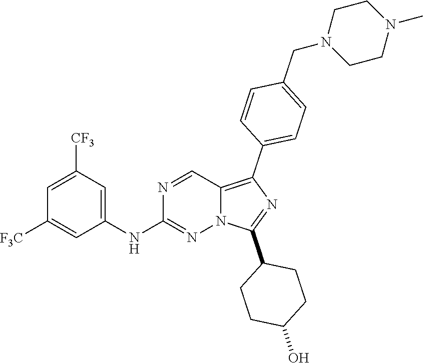 | +++ | + | + | ¹H NMR (400 MHz, DMSO-d₆) δ 10.54 (s, 1H), 9.50 (s, 1H), 8.46 (s, 2H), 8.01 (d, J = 8.2 Hz, 2H), 7.62 (s, 1H), 7.49 (d, J = 8.2 Hz, 2H), 4.04 (s, 2H), 3.54- 3.45 (m, 1H), 3.43-3.15 (m, 8H), 3.15-3.03 (m, 1H), 2.80 (s, 3H), 2.12-1.91 (m, 4H), 1.77-1.62 (m, 2H), 1.38-1.23 (m, 2H). MS (ESI) m/z calcd for C₃₁H₃₃F₆N7O: 634.27 [M + H]⁺; found 634.30. |

### Example 4—Syntheses 4-(6-((3-chloro-5-fluorophenyl)amino)-3-(4-((4-methylpiperazin-1-yl)methyl)phenyl)-1H-pyrazolo[3,4-d]pyrimidin-1-yl)cyclohexan-1-ol

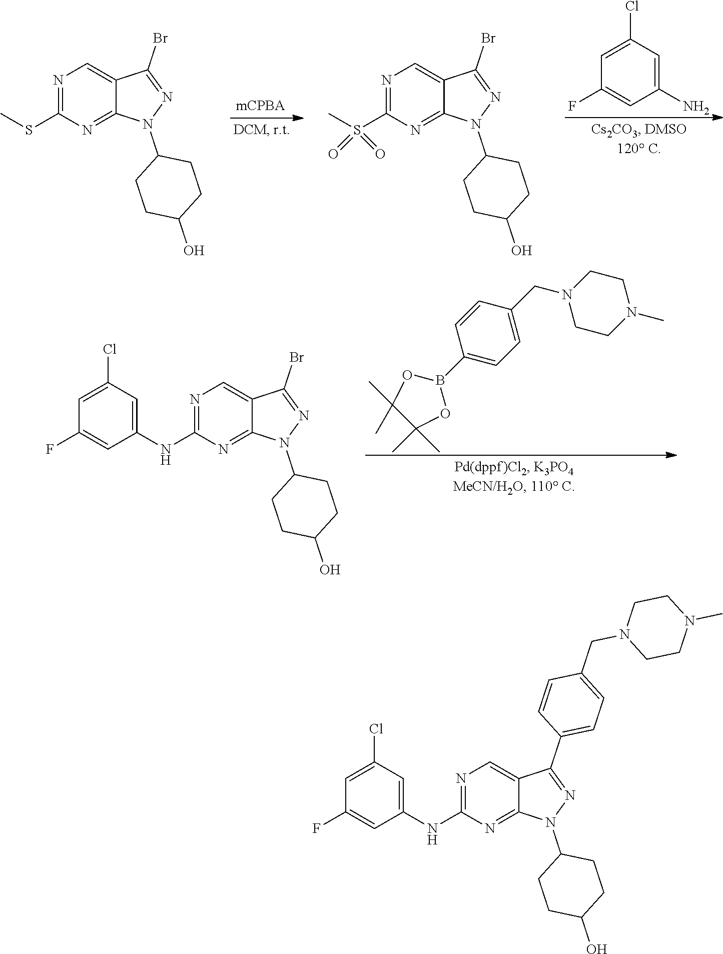

To a solution of 4-(3-bromo-6-(methylthio)-1H-pyrazolo[3,4-d]pyrimidin-1-yl)cyclohexan-1-ol (100.0 mg, 291.3 μmol) in CH₂Cl₂ was added m-CPBA (201 mg, 1.17 mmol). The reaction mixture was stirred at rt for 12 h, quenched with a saturated aqueous sodium bicarbonate solution (stir for 10 min), extracted with CH₂Cl₂ (3×). The combined organic phase was washed with brine, dried (Na₂SO₄), and concentrated to afford 4-(3-bromo-6-(methylsulfonyl)-1H-pyrazolo[3,4-d]pyrimidin-1-yl)cyclohexan-1-ol as a mixture of trans/cis isomers (109 mg, quant.). MS (ESI) m/z calcd for C₁₂H₁₅BrN₄O₃S: 375.00 [M+H]⁺; found 375.10.

To a solution of 4-(3-bromo-6-(methylsulfonyl)-1H-pyrazolo[3,4-d]pyrimidin-1-yl)cyclohexan-1-ol (100 mg, 0.27 mmol) and 3-chloro-5-fluoroaniline (78 mg, 0.53 mmol) in DMSO was added cesium carbonate (260 mg, 0.80 mmol). The reaction mixture was heated at 120° C. for 2 h, then quenched with a saturated aqueous NaCl solution at rt and extracted with ethyl acetate (3×). The combined organic phase was washed with brine, dried (Na₂SO₄), and concentrated. The residue was purified by an ISCO silica gel column to afford 4-(3-bromo-6-((3-chloro-5-fluorophenyl)amino)-1H-pyrazolo[3,4-d]pyrimidin-1-yl)cyclohexan-1-ol as a mixture of trans/cis isomers (87 mg, 74%). MS (ESI) m/z calcd for C₁₇H₁₆BrClFNO: 440.02 [M+H]⁺; found 440.10.

To a suspension of potassium phosphate tribasic (72 mg, 340 μmol), 4-(3-bromo-6-((3-chloro-5-fluorophenyl)amino)-1H-pyrazolo[3,4-d]pyrimidin-1-yl)cyclohexan-1-ol (50 mg, 113 μmol) and 1-methyl-4-(4-(4,4,5,5-tetramethyl-1,3,2-dioxaborolan-2-yl)benzyl) piperazine (39.5 mg, 125 μmol) in MeCN/H₂O (9 mL/1 mL) was added 1,1′-bis(diphenylphosphino) ferrocene-palladium (II) dichloride (12 mg, 17.0 μmol). The reaction mixture was degassed and then heated at 110° C. for 1 h under nitrogen atmosphere. After cooling to room temperature, the mixture was filtered through Celite and the filtration was collected. Solvents were removed under reduced pressure and the residue was purified by preparative HPLC with MeCN/H₂O as the eluents to afford the trans- and cis-isomers (15 mg & 5 mg respectively, 32% in total) as light yellow solids. trans-4-(6-((3-chloro-5-fluorophenyl)amino)-3-(4-((4-methylpiperazin-1-yl)methyl)phenyl)-1H-pyrazolo[3,4-d]pyrimidin-1-yl)cyclohexan-1-ol. ¹H NMR (400 MHz, DMSO-d₆) δ 10.41 (s, 1H), 9.42 (s, 1H), 8.07 (d, J=7.9 Hz, 2H), 7.95 (t, J=2.0 Hz, 1H), 7.75 (dt, J=11.9, 2.2 Hz, 1H), 7.56 (d, J=7.9 Hz, 2H), 6.99 (dt, J=8.4, 2.1 Hz, 1H), 4.59 (tt, J=11.5, 3.6 Hz, 1H), 4.03 (s, 2H), 3.62 (tt, J=10.8, 3.6 Hz, 1H), 3.54-2.92 (m, 8H), 2.83 (s, 3H), 2.36-1.97 (m, 6H), 1.52-1.34 (m, 2H). MS (ESI) m/z calcd for C₂₉H₃₃ClFN₇O: 550.24 [M+H]⁺; found 550.30. cis-4-(6-((3-chloro-5-fluorophenyl)amino)-3-(4-((4-methylpiperazin-1-yl)methyl)phenyl)-1H-pyrazolo[3,4-d]pyrimidin-1-yl)cyclohexan-1-ol. MS: [M+H]+=550.3 found. ¹H NMR (400 MHz, DMSO-d₆) δ 10.42 (s, 1H), 9.44 (s, 1H), 8.06 (d, J=7.8 Hz, 2H), 7.93 (t, J=2.1 Hz, 1H), 7.78 (dt, J=11.9, 2.1 Hz, 1H), 7.52 (d, J=7.8 Hz, 2H), 7.00 (dt, J=8.5, 2.2 Hz, 1H), 4.65 (tt, J=11.8, 4.2 Hz, 1H), 3.84 (s, 2H), 3.68-3.59 (m, 1H), 3.53-2.90 (m, 8H), 2.80 (s, 3H), 2.54 (s, 1H), 2.09-1.81 (m, 4H), 1.54-1.36 (m, 2H), 1.35-1.08 (m, 2H). MS (ESI) m/z calcd for C₂₉H₃₃ClFN₇O: 550.24 [M+H]⁺; found 550.30.

Additional compounds can be prepared following the synthesis procedure described in Example 4 using the corresponding reagents.

Table 5 describes compounds prepared following procedures described in Example 4 using appropriate reagents. Biological assays were performed as described in Example 5 (Note: Mer IC₅₀: ++++ means <10 nM; +++ means between 10-100 nM, ++ means between 100 nM-1 μM; + means between 1-30 μM; − means inactive).

TABLE 5

| | | | | | Physical Data |
|---|---|---|---|---|---|
| Cpd. | | TYRO3 | MERTK | AXL | MS m/z (M + 1) or/and ¹H |
| No. | Structure | IC₅₀ | IC₅₀ | IC₅₀ | NMR |
| 43 | 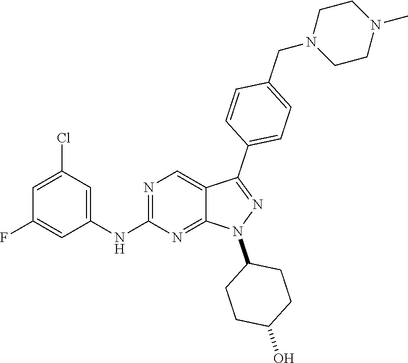 | ++++ | ++++ | ++ | ¹H NMR (400 MHz, DMSO-d₆) δ 10.41 (s, 1H), 9.42 (s, 1H), 8.07 (d, J = 7.9 Hz, 2H), 7.95 (t, J = 2.0 Hz, 1H), 7.75 (dt, J = 11.9, 2.2 Hz, 1H), 7.56 (d, J = 7.9 Hz, 2H), 6.99 (dt, J = 8.4, 2.1 Hz, 1H), 4.59 (tt, J = 11.5, 3.6 Hz, 1H), 4.03 (s, 2H), 3.62 (tt, J = 10.8, 3.6 Hz, 1H), 3.54-2.92 (m, 8H), 2.83 (s, 3H), 2.36-1.97 (m, 6H), 1.52-1.34 (m, 2H). MS (ESI) m/z calcd for C₂₉H₃₃ClFN₇O: 550.24 [M + H]⁺; found 550.30. |
| 44 | 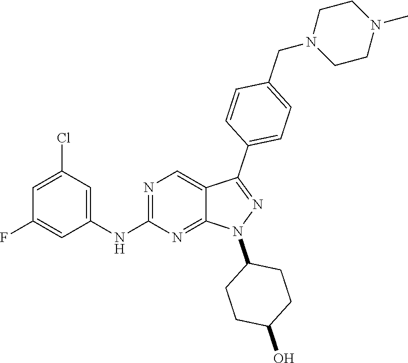 | +++ | +++ | ++ | ¹H NMR (400 MHz, DMSO-d₆) δ 10.42 (s, 1H), 9.44 (s, 1H), 8.06 (d, J = 7.8 Hz, 2H), 7.93 (t, J = 2.1 Hz, 1H), 7.78 (dt, J = 11.9, 2.1 Hz, 1H), 7.52 (d, J = 7.8 Hz, 2H), 7.00 (dt, J = 8.5, 2.2 Hz, 1H), 4.65 (tt, J = 11.8, 4.2 Hz, 1H), 3.84 (s, 2H), 3.68-3.59 (m, 1H), 3.53-2.90 (m, 8H), 2.80 (s, 3H), 2.54 (s, 1H), 2.09-1.81 (m, 4H), 1.54-1.36 (m, 2H), 1.35- 1.08 (m, 2H). MS (ESI) m/z calcd for C₂₉H₃₃ClFN₇O: 550.24 [M + H]⁺; found 550.30. |
| 45 | 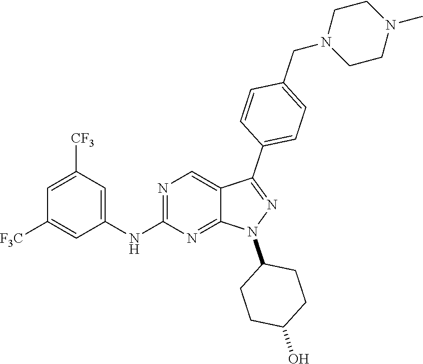 | ++++ | + | ++ | ¹H NMR (400 MHz, DMSO-d₆) δ 10.69 (s, 1H), 9.44 (s, 1H), 8.63 (s, 2H), 8.10 (d, J = 8.0 Hz, 2H), 7.61 (d, J = 8.0 Hz, 2H), 7.59 (s, 1H), 4.59 (tt, J = 10.2, 4.9 Hz, 1H), 4.27 (s, 2H), 3.65-3.54 (m, 1H), 3.52-3.03 (m, 8H), 2.87 (s, 3H), 2.14-1.93 (m, 6H), 1.51- 1.29 (m, 2H). MS (ESI) m/z calcd for C₃₁H₃₃F₆N₇O: 634.27 [M + H]⁺; found 634.30. |
| 46 | 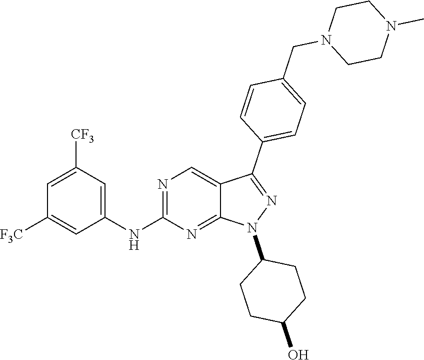 | ++++ | − | + | ¹H NMR (400 MHz, DMSO-d₆) δ 10.73 (s, 1H), 9.49 (s, 1H), 8.66 (s, 2H), 8.13 (d, J = 8.2 Hz, 3H), 7.64 (s, 1H), 7.61 (d, J = 8.2 Hz, 2H), 4.67 (tt, J = 11.9, 3.9 Hz, 1H), 4.20 (s, 2H), 3.61 (tt, J = 10.9, 4.2 Hz, 1H), 3.54-2.93 (m, 8H), 2.85 (s, 3H), 2.07 (s, 1H), 2.03-1.78 (m, 4H), 1.50-1.32 (m, 2H), 1.31-1.14 (m, 2H). MS (ESI) m/z calcd for C₃₁H₃₃F₆N₇O: 634.27: [M + H]⁺; found 634.30. |

### Example 5—Biological Assays

### Microfluidic Capillary Electrophoresis (MCE) Assay

Activity assays were performed in a 384 well, polypropylene microplate in a final volume of 50 μL of 50 mM Hepes, Ph 7.4 containing 10 mM MgCl₂, 1.0 mM DTT, 0.01% Triton X-100, 0.1% Bovine Serum Albumin (BSA), containing 1.0 μM fluorescent substrate (Table 6) and ATP at the Km for each enzyme (Table S1). All reactions were terminated by addition of 20 μL of 70 mM EDTA. After a 180 min incubation, phosphorylated and unphosphorylated substrate peptides (Table 6) were separated in buffer supplemented with 1× CR-8 on a LabChip EZ Reader equipped with a 12-sipper chip. Data were analyzed using EZ Reader software.

TABLE 6
Assay conditions for MCE assays

| | | Kinase | ATP |
|---|---|---|---|
| Kinase | Peptide Substrate | (nM) | (uM) |
| Mer | 5-FAM-EFPIYDFLPAKKK-CONH₂ | 1.7 | 22.3 |
| Axl | 5-FAM-KKKKEEIYFFF-CONH₂ | 16 | 200 |
| Tyro3 | 5-FAM-EFPIYDFLPAKKK-CONH₂ | 5 | 40 |
| Flt3 | 5-FAM-KKKKEEIYFFF-CONH₂ | 0.3 | 275 |

### Example 6—Syntheses

### trans-4-(2-((3,5-Bis(trifluoromethyl)phenyl)amino)-5-(4-((1-methylpiperidin-4-yl)oxy)phenyl)-7H-pyrrolo[2,3-d]pyrimidin-7-yl)cyclohexan-1-ol

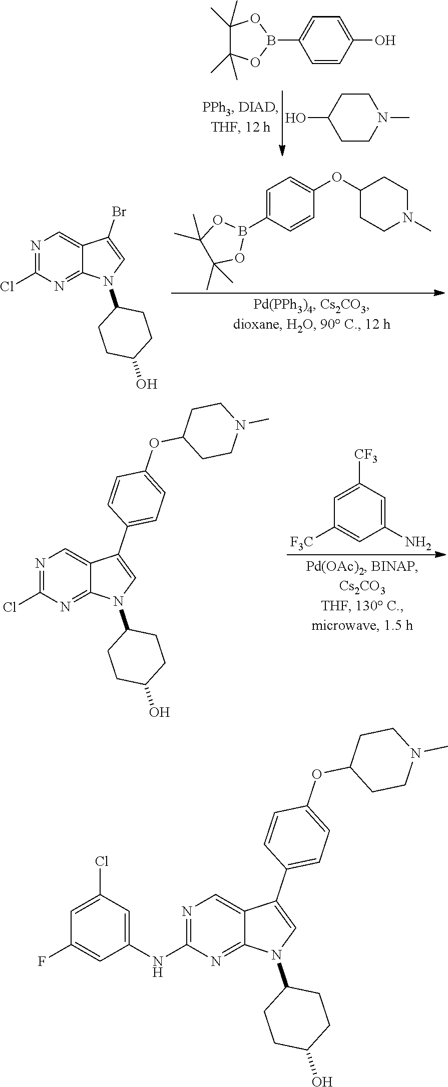

To a solution of 4-(4,4,5,5-tetramethyl-1,3,2-dioxaborolan-2-yl) phenol (2.00 g, 9.1 mmol), triphenylphosphine (3.10 g, 11.8 mmol) and 1-methylpiperidin-4-ol (1.20 g, 10.0 mmol) in dry THF (50 mL) was added diisopropyl (E)-diazene-1,2-dicarboxylate (2.40 g, 11.8 mmol) at 0° C. The reaction mixture was stirred at 0° C. for 0.5 h, then warmed to rt and stirred for 12 h, then quenched with water (20 mL) and extracted with EtOAc (60 mL, 2×). The combined organic layers were washed with brine (10 mL), dried (Na₂SO₄), and concentrated under reduced pressure. The residue was purified by an ISCO silica gel column (petroleum ether/ethyl acetate=10/1 to 5/1) to afford the desired product (1.20 g, 43%) as a white solid. ¹H NMR (400 MHz, CD₃OD) δ 7.66 (d, J=8.8 Hz, 2H), 6.91 (d, J=8.8 Hz, 2H), 4.56-4.40 (m, 2H), 2.77-2.66 (m, 2H), 2.44-2.34 (m, 2H), 2.09-1.95 (m, 2H), 1.87-1.75 (m, 2H), 1.32 (s, 9H). MS (ESI) m/z calcd for C₁₈H₂₉BNO₃: 318.22 [M+H]⁺; found 318.20. LC-MS: >95% purity.

To a solution of trans-4-(5-bromo-2-chloro-7H-pyrrolo[2,3-d]pyrimidin-7-yl)cyclohexan-1-ol (0.6 g, 1.8 mmol) in 1,4-dioxane (20 mL) and water (2 mL) was added 1-methyl-4-(4-(4,4,5,5-tetramethyl-1,3,2-dioxaborolan-2-yl) phenoxy) piperidine (0.60 g, 2.0 mmol), cesium carbonate (1.20 g, 3.6 mmol), and Pd(PPh₃)₄ (0.10 g, 91 μmol). The reaction mixture was heated under nitrogen atmosphere at 90° C. for 12 h, then cooled to rt, quenched with H₂O, and extracted with EtOAc (30 mL, 3×). The combined organic layers were washed with brine, dried (Na₂SO₄), and concentrated under reduced pressure. The residue was purified by an ISCO reversed column (methanol/water+0.1% HCl, 10-100%) to afford a slight yellow solid, which was triturated with methanol. The solid was filtered to afford the desired product trans-4-(2-chloro-5-(4-((1-methylpiperidin-4-yl)oxy)phenyl)-7H-pyrrolo[2,3-d]pyrimidin-7-yl)cyclohexan-1-ol (0.60 g, 70%) as a yellow solid. ¹H NMR (400 MHz, CDCl₃) δ 8.98 (s, 1H), 7.52 (d, J=8.6 Hz, 2H), 7.35 (s, 1H), 7.01 (d, J=8.5 Hz, 2H), 4.79-4.71 (m, 1H), 3.86-3.73 (m, 2H), 3.38 (d, J=10.5 Hz, 3H), 3.18 (q, J=10.4 Hz, 2H), 2.82 (s, 3H), 2.67 (t, J=13.3 Hz, 2H), 2.30-2.09 (m, 7H), 1.99-1.81 (m, 2H), 1.71-1.59 (m, 3H). MS (ESI) m/z calcd for C₂₄H₃₀ClN₄O₂: 441.20 [M+H]⁺; found 441.20. LC-MS: >95% purity.

To a solution of trans-4-(2-chloro-5-(4-((1-methylpiperidin-4-yl)oxy)phenyl)-7H-pyrrolo[2,3-d]pyrimidin-7-yl)cyclohexan-1-ol (60 mg, 0.1 mmol) and 3,5-dichloroaniline (94 mg, 0.40 mmol) in dry THF (10 mL) were added palladium (II) acetate (1.5 mg, 7 μmol), Sphos (3.0 mg, 7.0 μmol), and Cs₂CO₃ (89 mg, 0.3 mmol). The reaction mixture was heated under nitrogen atmosphere and microwave radiation at 130° C. for 1.5 h, then quenched with water and extracted with EtOAc (15 mL, 3×). The combined organic layers were washed with water and brine, dried (Na₂SO₄), and concentrated. The residue was purified by an HPLC (Acetonitrile/water+0.05% TFA, 8-100%) to afford a slight yellow solid, which was converted to HCl salt the treatment of a 4 N HCl solution in 1,4-dioxane followed by lyophilization to provide the desired product trans-4-(2-((3,5-bis(trifluoromethyl)phenyl)amino)-5-(4-((1-methylpiperidin-4-yl)oxy)phenyl)-7H-pyrrolo[2,3-d]pyrimidin-7-yl)cyclohexan-1-ol (26 mg, 30%). ¹H NMR (400 MHz, DMSO-d₆) δ 10.49 (s, 1H), 9.12 (d, J=4.7 Hz, 1H), 8.61 (s, 2H), 7.87 (d, J=2.3 Hz, 2H), 7.70 (t, J=8.4 Hz, 2H), 7.57 (s, 2H), 7.09 (dd, J=20.1, 8.9 Hz, 2H), 4.64-4.46 (m, 1H), 3.62-3.53 (m, 1H), 3.47 (d, J=10.9 Hz, 1H), 3.29 (d, J=9.7 Hz, 1H), 3.23-3.04 (m, 4H), 2.75 (dd, J=11.1, 4.8 Hz, 3H), 2.29-2.12 (m, 2H), 2.10-1.89 (m, 9H), 1.38 (t, J=7.7 Hz, 2H). MS (ESI) m/z calcd for C₃₂H₃₄F₆N₅O₂: 634.25 [M+H]⁺; found 634.20. LC-MS: >96% purity.

Table 7 describes compounds prepared following procedures described in Example 6 using appropriate reagents. (Note: Mer IC₅₀: ++++ means <10 nM; +++ means between 10-100 nM, ++ means between 100 nM-1 μM; + means between 1-30 μM; − means inactive.)

TABLE 7

| | | | | | Physical Data |
|---|---|---|---|---|---|
| | | TYRO3 | MERTK | AXL | MS m/z (M + 1) or/and ¹H |
| Compound | Structure | IC₅₀ | IC₅₀ | IC₅₀ | NMR |
| 150 | 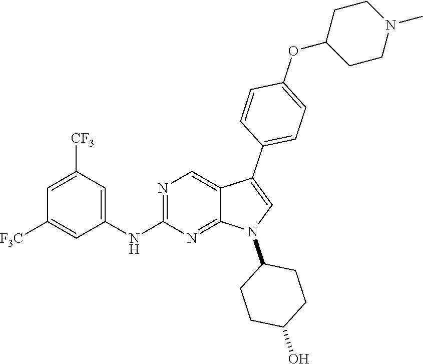 | +++ | + | + | MS (ESI) m/z calcd for C₃₂H₃₄F₆N₅O₂: 634.25 [M + H]⁺; found 634.20. ¹H NMR (400 MHz, DMSO-d₆) δ 10.49 (s, 1H), 9.12 (d, J = 4.7 Hz, 1H), 8.61 (s, 2H), 7.87 (d, J = 2.3 Hz, 2H), 7.70 (t, J = 8.4 Hz, 2H), 7.57 (s, 2H), 7.09 (dd, J = 20.1, 8.9 Hz, 2H), 4.64- 4.46 (m, 1H), 3.62-3.53 (m, 1H), 3.47 (d, J = 10.9 Hz, 1H), 3.29 (d, J = 9.7 Hz, 1H), 3.23- 3.04 (m, 4H), 2.75 (dd, J = 11.1, 4.8 Hz, 3H), 2.29-2.12 (m, 2H), 2.10-1.89 (m, 9H), 1.38 (t, J = 7.7 Hz, 2H). |
| 60 | 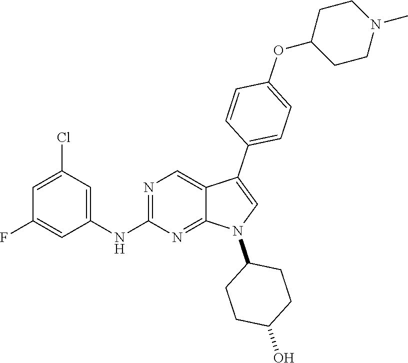 | ++++ | ++++ | +++ | MS (ESI) m/z calcd for C₃₀H₃₄ClFN₅O₂: 550.23 [M + H]⁺; found 550.20. ¹H NMR (400 MHz, DMSO-d₆) δ 10.44 (s, 1H), 9.22 (s, 1H), 8.63 (s, 2H), 8.09 (s, 1H), 7.89 (d, J = 8.5 Hz, 2H), 7.57 (s, 1H), 7.53 (d, J = 8.5 Hz, 2H), 4.62-4.50 (m, 1H), 3.60- 3.53 (m, 6H), 3.46-3.38 (m, 3H), 3.16-3.04 (m, 2H), 2.78 (s, 3H), 2.01 (d, J = 6.7 Hz, 6H), 1.49-1.32 (m, 2H). |

### Example 7—Syntheses trans-4-(2-((3,5-Bis(trifluoromethyl)phenyl)amino)-5-(4-((tetrahydro-2H-pyran-4-yl)amino)phenyl)-7H-pyrrolo[2,3-d]pyrimidin-7-yl)cyclohexan-1-ol

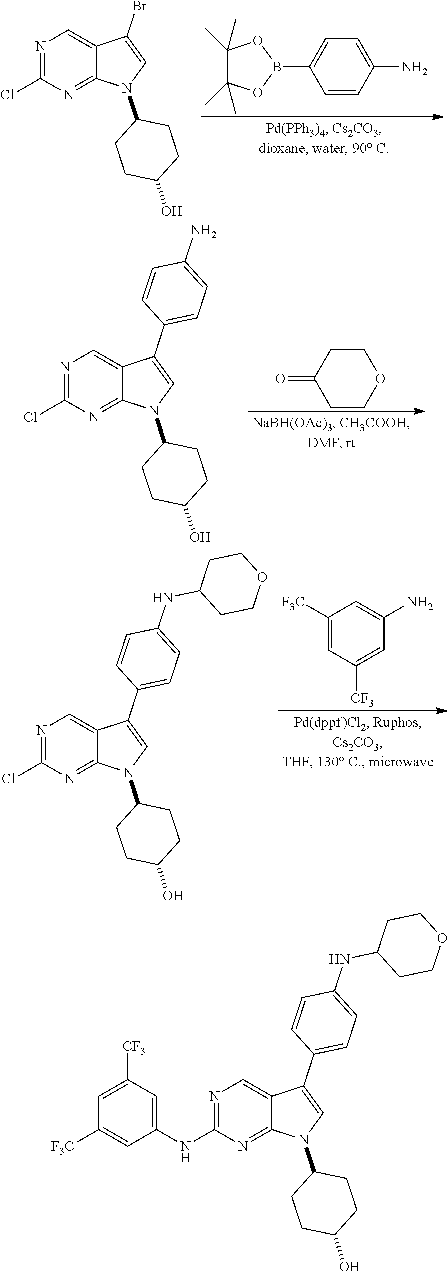

To a solution of trans-4-(5-bromo-2-chloro-7H-pyrrolo[2,3-d]pyrimidin-7-yl)cyclohexan-1-ol (0.50 g, 1.50 mmol) in 1,4-dioxane (10 mL) and water (1.0 mL) were added 4-(4,4,5,5-tetramethyl-1,3,2-dioxaborolan-2-yl) aniline (0.70 g, 3.0 mmol), cesium carbonate (1.50 g, 4.60 mmol), and Pd(PPh₃)₄ (88 mg, 76 μmol). The reaction mixture was heated under nitrogen atmosphere at 90° C. for 16 h, then cooled to rt, quenched with H₂O, and extracted with EtOAc (20 mL, 3×). The combined organic layers were washed with brine, dried over Na₂SO₄, and concentrated under reduced pressure. The residue was purified by an ISCO reversed column (acetonitrile/water+0.1% HCl, 10-100%) to afford a slight yellow solid, which was triturated with methanol. The solid was filtered to afford the desired product trans-4-(5-(4-aminophenyl)-2-chloro-7H-pyrrolo[2,3-d]pyrimidin-7-yl)cyclohexan-1-ol (0.34 g, 66%) as a yellow solid. ¹H NMR (400 MHz, DMSO-d₆) δ 9.24 (s, 1H), 8.25 (s, 1H), 7.83 (d, J=8.1 Hz, 2H), 7.32 (d, J=8.1 Hz, 2H), 4.64-4.55 (m, 1H), 3.17-3.16 (m, 1H), 2.02-1.90 (m, 6H), 1.50-1.38 (m, 2H). MS (ESI) m/z calcd for C₁₈H₂₀ClN₄O: 343.13 [M+H]⁺; found 343.20. LC-MS: >95% purity.

To a mixture of trans-4-(5-(4-aminophenyl)-2-chloro-7H-pyrrolo[2,3-d]pyrimidin-7-yl)cyclohexan-1-ol (150 mg, 0.40 mmol) in DMF (5.0 mL) was added acetic acid (53 mg, 0.90 mmol), tetrahydro-4H-pyran-4-one (88 mg, 0.90 mmol), and sodium triacetoxyborohydride (187 mg, 0.90 mmol). The reaction mixture was stirred at 25° C. for 12 h, quenched with MeOH, and filtrated. The filtrate was concentrated and the residue was purified by an ISCO silica gel column (hexane/ethyl acetate=2/1 to 1/1) to yield the desired product trans-4-(2-chloro-5-(4-((tetrahydro-2H-pyran-4-yl)amino)phenyl)-7H-pyrrolo[2,3-d]pyrimidin-7-yl)cyclohexan-1-ol (118 mg, 63%) as a yellow solid. ¹H NMR (400 MHz, DMSO-d₆) δ 9.22 (s, 1H), 8.24 (s, 1H), 7.83 (d, J=8.1 Hz, 2H), 7.41 (d, J=8.1 Hz, 2H), 4.62-4.51 (m, 1H), 3.91-3.84 (m, 2H), 3.34-3.25 (m, 3H), 2.02-1.79 (m, 9H), 1.75-1.61 (m, 2H), 1.48-1.34 (m, 2H). MS (ESI) for [M+H]⁺(C₂₃H₂₈N₄O₂⁺): calcd. m/z 427.18; found m/z 427.20. LC/MS: 96% purity.

To a solution of trans-4-(2-chloro-5-(4-((tetrahydro-2H-pyran-4-yl)amino)phenyl)-7H-pyrrolo[2,3-d]pyrimidin-7-yl)cyclohexan-1-ol (50 mg, 0.10 mmol) and 3,5-dichloroaniline (80 mg, 0.40 mmol) in dry THF (10 mL) were added palladium (II) acetate (3.0 mg, 12 μmol), BINAP (7.5 mg, 12 μmol), and Cs₂CO₃ (117 mg, 0.60 mmol). The reaction mixture was heated under nitrogen atmosphere and microwave radiation at 130° C. for 0.5 h, then quenched with water and extracted with EtOAc (20 mL, 3×). The combined organic layers were washed with water and brine, dried (Na₂SO₄), and concentrated. The residue was purified by an HPLC (Acetonitrile/water+0.05% TFA, 8-100%) to afford a slight yellow solid, which was converted to HCl salt the treatment of a 4.0 N HCl solution in 1,4-dioxane followed by lyophilization to provide the desired compound trans-4-(2-((3,5-bis(trifluoromethyl)phenyl)amino)-5-(4-((tetrahydro-2H-pyran-4-yl)amino)phenyl)-7H-pyrrolo[2,3-d]pyrimidin-7-yl)cyclohexan-1-ol (85 mg, 57%) as a yellow solid. ¹H NMR (400 MHz, DMSO-d₆) δ 10.39 (s, 1H), 9.20 (s, 1H), 8.63 (s, 2H), 8.00 (s, 1H), 7.89 (d, J=8.1 Hz, 2H), 7.57 (s, 1H), 7.47 (d, J=8.1 Hz, 2H), 4.59-4.52 (m, 1H), 3.96-3.88 (m, 2H), 3.73-3.63 (m, 2H), 3.38-3.27 (m, 2H), 2.06-1.91 (m, 6H), 1.87 (dd, J=13.2, 3.7 Hz, 2H), 1.79-1.66 (m, 2H), 1.44-1.35 (m, 2H). MS (ESI) m/z calcd for C₃₁H₃₂F₆N₅O₂: 620.24 [M+H]⁺; found 620.20. LC-MS: >97% purity.

Table 8 describes compounds prepared following procedures described in Example 7 using appropriate reagents. (Note: Mer IC₅₀: ++++ means <10 nM; +++ means between 10-100 nM, ++ means between 100 nM-1 μM; + means between 1-30 μM; − means inactive.)

TABLE 8

| | | TYRO3 | MERTK | AXL | Physical Data |
|---|---|---|---|---|---|
| Compound | Structure | IC₅₀ | IC₅₀ | IC₅₀ | MS m/z (M + 1) or/and ¹H NMR |
| 51 | 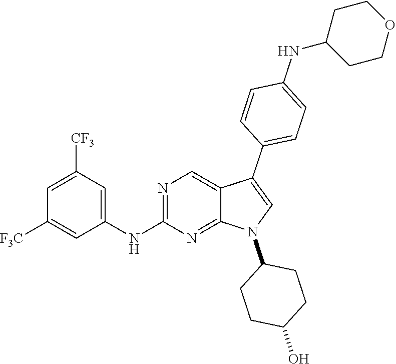 | +++ | + | | MS (ESI) m/z calcd for C₃₁H₃₂F₆N₅O₂: 620.24 [M + H]⁺; found 620.20. ¹H NMR (400 MHz, DMSO-d₆) δ 10.39 (s, 1H), 9.20 (s, 1H), 8.63 (s, 2H), 8.00 (s, 1H), 7.89 (d, J = 8.1 Hz, 2H), 7.57 (s, 1H), 7.47 (d, J = 8.0 Hz, 2H), 4.59-4.52 (m, 1H), 3.96-3.88 (m, 2H), 3.73-3.63 (m, 2H), 3.38- 3.27 (m, 2H), 2.06-1.91 (m, 6H), 1.87 (dd, J = 13.2, 3.7 Hz, 2H), 1.79-1.66 (m, 2H), 1.44-1.35 (m, 2H). |
| 52 | 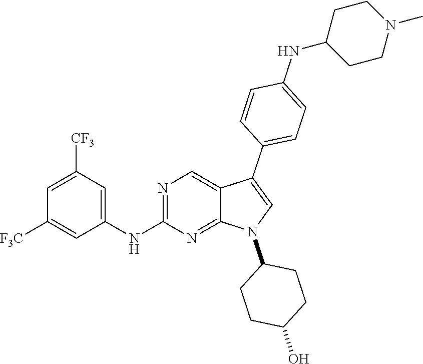 | ++++ | + | | MS (ESI) m/z calcd for C₃₂H₃₄F₆N₆O: 633.27 [M + H]⁺; found 633.30. ¹H NMR (400 MHz, CD₃OD) δ 8.88 (s, 1H), 8.52 (s, 2H), 7.48-7.38 (m, 4H), 6.78 (d, J = 8.1 Hz, 2H), 4.69- 4.58 (m, 1H), 3.80-3.67 (m, 1H), 3.63- 3.55 (m, 2H), 3.18 (t, J = 12.8 Hz, 2H), 2.90 (s, 3H), 2.33 (d, J = 14.2 Hz, 2H), 2.18-2.05 (m, 4H), 2.04-1.93 (m, 2H), 1.78-1.64 (m, 2H), 1.63- 1.50 (m, 2H). |
| 56 | 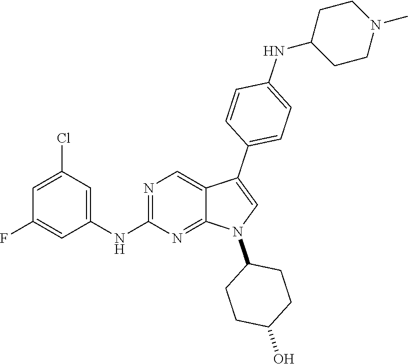 | ++++ | ++++ | | MS (ESI) m/z calcd for C₃₀H₃₅ClFN₆O: 549.30 [M + H]⁺; found 549.30. ¹H NMR (400 MHz, DMSO-d₆) δ 10.32 (s, 1H), 10.05 (s, 1H), 9.03 (s, 1H), 7.88 (s, 1H), 7.74-7.66 (m, 2H), 7.63-7.51 (m, 2H), 7.03-6.81 (m, 3H), 4.50-4.37 (m, 1H), 3.75-3.68 (m, 2H), 3.27-3.20 (m, 2H), 3.00 (q, J = 11.4 Hz, 2H), 2.71 (s, 3H), 2.10 (d, J = 13.5 Hz, 2H), 1.99 (dt, J = 17.3, 4.9 Hz, 7H), 1.85-1.71 (m, 2H), 1.45- 1.30 (m, 2H). |

### Example 8—Syntheses (4-(2-((3,5-Bis(trifluoromethyl)phenyl)amino)-7-(trans-4-hydroxycyclohexyl)-7H-pyrrolo[2,3-d]pyrimidin-5-yl)phenyl)(4-methylpiperidin-1-yl)methanone

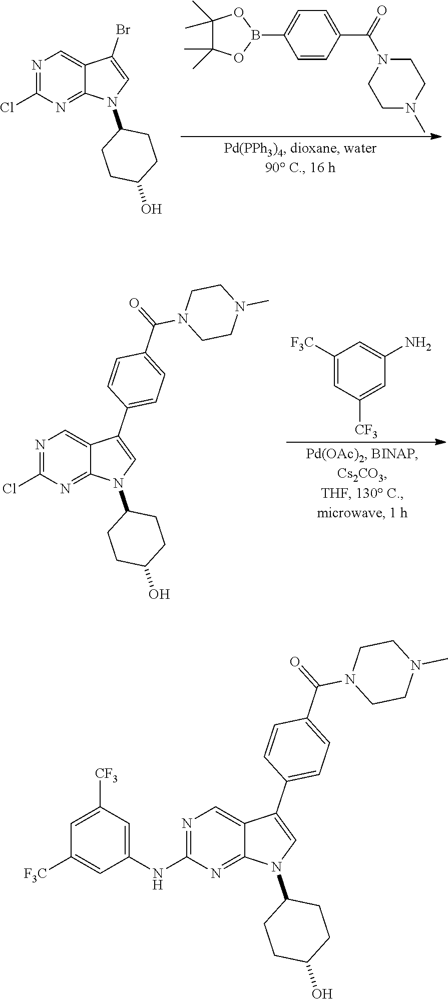

To a solution of trans-4-(5-bromo-2-chloro-7H-pyrrolo[2,3-d]pyrimidin-7-yl)cyclohexan-1-ol (5.0 g, 15.2 mmol) in 1,4-dioxane (50 mL) and water (5.0 mL) was added (4-methylpiperazin-1-yl) (4-(4,4,5,5-tetramethyl-1,3,2-dioxaborolan-2-yl)phenyl)methanone (5.5 g, 16.6 mmol), cesium carbonate (14.9 g, 45.6 mmol), and Pd(PPh₃)₄ (0.90 g, 0.80 mmol). The reaction mixture was heated under nitrogen atmosphere at 90° C. for 16 h, then cooled to rt, quenched with H₂O, and extracted with EtOAc (30 mL, 3×). The combined organic layers were washed with brine, dried (MgSO₄), and concentrated under reduced pressure. The residue was purified by an ISCO reversed column (acetonitrile/water+0.1% HCl, 10-100%) to afford a slight yellow solid, which was triturated with methanol. The solid was filtered to afford the desired product (4-(2-chloro-7-(trans-4-hydroxycyclohexyl)-7H-pyrrolo[2,3-d]pyrimidin-5-yl)phenyl) (4-methylpiperazin-1-yl)methanone (4.0 g, 58%) as a yellow solid. ¹H NMR (400 MHz, DMSO-d₆) δ 11.28 (s, 1H), 9.30 (s, 1H), 8.38 (s, 1H), 7.93-7.87 (m, 2H), 7.58-7.51 (m, 2H), 4.67-4.54 (m, 1H), 3.44-3.40 (m, 4H), 3.16-3.06 (m, 2H), 2.77 (d, J=3.9 Hz, 3H), 2.07-1.90 (m, 7H), 1.45 (tt, J=13.6, 7.7 Hz, 2H). MS (ESI) m/z calcd for C₂₄H₂₉ClN₅O₂: 454.20 [M+H]⁺; found 454.20. LC-MS: >95% purity.

To a solution of (4-(2-chloro-7-(trans-4-hydroxycyclohexyl)-7H-pyrrolo[2,3-d]pyrimidin-5-yl)phenyl) (4-methylpiperazin-1-yl)methanone (100 mg, 0.20 mmol) and 3,5-dichloroaniline (152 mg, 0.70 mmol) in dry THF (8.0 mL) was added palladium (II) acetate (3.0 mg, 11 μmol), BINAP (7.0 mg, 11 μmol), and Cs₂CO₃ (215 mg, 0.70 mmol). The reaction mixture was heated under nitrogen atmosphere and microwave radiation at 130° C. for 1.0 h, then quenched with water, and extracted with EtOAc (10 mL, 3×). The combined organic layers were washed with water and brine, dried (Na₂SO₄), and concentrated. The residue was purified by an ISCO reversed column (methanol/water+0.1% HCl, 10-100%) to afford crude product which was recrystallized from methanol to afford the title compound (102 mg, 0.16 mmol, 72%) as a yellow solid. ¹H NMR (400 MHz, DMSO-d₆) δ 10.08 (s, 1H), 9.19 (s, 1H), 8.02 (s, 1H), 7.94 (s, 1H), 7.87 (d, J=8.5 Hz, 2H), 7.74 (dt, J=12.2, 2.2 Hz, 1H), 7.52 (d, J=8.4 Hz, 2H), 6.94-6.89 (m, 1H), 4.57-4.45 (m, 1H), 3.67-3.56 (m, 2H), 3.44-3.31 (m, 6H), 3.15-3.04 (m, 3H), 2.78 (s, 3H), 2.10-1.98 (m, 7H), 1.53-1.35 (m, 2H). MS (ESI) m/z calcd for C₃₂H₃₃F₆N₆O₂: 647.25 [M+H]⁺; found 647.20. LC-MS: >97% purity.

Table 9 describes compounds prepared following procedures described in Example 8 using appropriate reagents. (Note: Mer IC₅₀: ++++ means <10 nM; +++ means between 10-100 nM, ++ means between 100 nM-1 μM; + means between 1-30 μM; − means inactive.)

TABLE 9

| | | TYRO3 | MERTK | AXL | Physical Data |
|---|---|---|---|---|---|
| Compound | Structure | IC₅₀ | IC₅₀ | IC₅₀ | MS m/z (M + 1) or/and ¹H NMR |
| 155 | 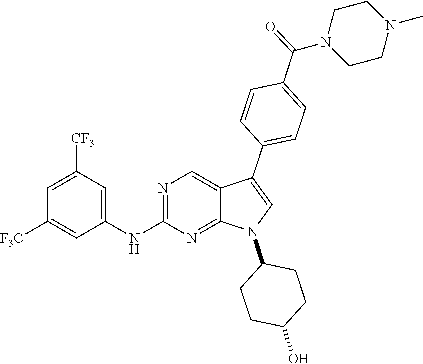 | ++++ | + | ++ | MS (ESI) m/z calcd for C₃₂H₃₃F₆N₆O₂: 647.25 [M + H]⁺; found 647.20. ¹H NMR (400 MHz, DMSO-d₆) δ 10.08 (s, 1H), 9.19 (s, 1H), 8.02 (s, 1H), 7.94 (s, 1H), 7.87 (d, J = 8.5 Hz, 2H), 7.74 (dt, J = 12.2, 2.2 Hz, 1H), 7.52 (d, J = 8.4 Hz, 2H), 6.94-6.89 (m, 1H), 4.57-4.45 (m, 1H), 3.67-3.56 (m, 2H), 3.44-3.31 (m, 6H), 3.15-3.04 (m, 3H), 2.78 (s, 3H), 2.10-1.98 (m, 7H), 1.53-1.35 (m, 2H). |
| 156 | 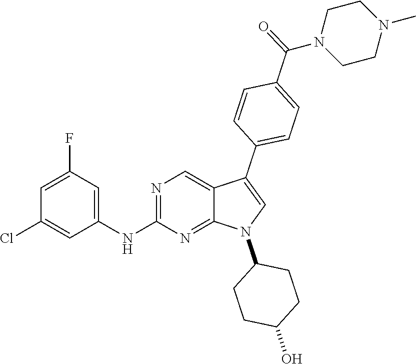 | ++++ | ++++ | +++ | MS (ESI) m/z calcd for C₃₀H₃₃ClFN₆O₂: 647.25 [M + H]⁺; found 647.20. ¹H NMR (400 MHz, DMSO-d₆) δ 11.00 (s, 1H), 10.05 (s, 1H), 9.15 (s, 1H), 7.98 (s, 1H), 7.95- 7.88 (m, 1H), 7.83 (d, J = 8.3 Hz, 2H), 7.70 (dt, J = 12.1, 2.2 Hz, 1H), 7.49 (d, J = 8.3 Hz, 2H), 6.88 (dt, J = 8.5, 2.2 Hz, 1H), 4.48 (dt, J = 10.6, 5.3 Hz, 1H), 3.62-3.51 (m, 3H), 3.15-2.99 (m, 3H), 2.75 (d, J = 3.9 Hz, 4H), 2.05-1.90 (m, 7H), 1.47- 1.34 (m, 2H). |
| 62 | 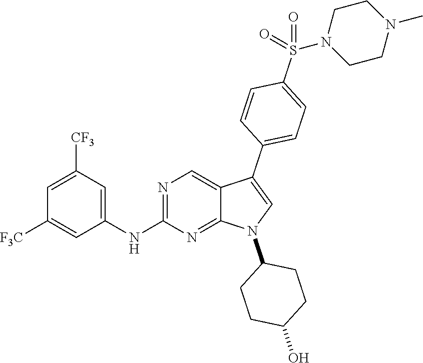 | ++++ | ++ | | MS (ESI) m/z calcd for C₃₁H₃₃F₆N₆O₃S: 683.21 [M + H]⁺; found 683.20. ¹H NMR (400 MHz, DMSO-d₆) δ 10.58 (s, 1H), 10.41 (s, 1H), 9.28 (s, 1H), 8.64 (s, 2H), 8.24 (s, 1H), 8.11 (d, J = 8.5 Hz, 2H), 7.80 (d, J = 8.5 Hz, 2H), 7.57 (s, 1H), 4.65-4.51 (m, 1H), 3.82 (d, J = 12.5 Hz, 2H), 3.66- 3.55 (m, 2H), 3.23-3.10 (m, 2H), 2.79-2.64 (m, 5H), 2.09-1.93 (m, 6H), 1.48-1.34 (m, 2H). |
| 63 | 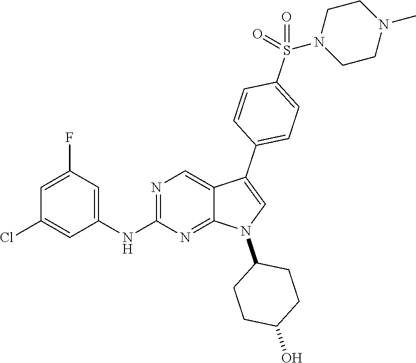 | ++++ | ++++ | | MS (ESI) m/z calcd for C₂₉H₃₃ClFN₆O₃S: 599.19 [M + H]⁺; found 599.20. ¹H NMR (400 MHz, DMSO-d₆) δ 10.08 (s, 1H), 9.25 (s, 1H), 8.17 (s, 1H), 8.09 (d, J = 8.2 Hz, 2H), 7.95 (s, 1H), 7.80 (d, J = 8.3 Hz, 2H), 7.77- 7.72 (m, 1H), 6.95-6.90 (m, 1H), 4.58-4.48 (m, 1H), 3.82 (d, J = 12.8 Hz, 2H), 3.68-3.55 (m, 2H), 3.20- 3.09 (m, 2H), 2.77 (s, 3H), 2.66 (s, 2H), 2.10-1.99 (m, 6H), 1.50-1.36 (m, 2H). |

### Example 9—Syntheses N-(3,5-Bis(trifluoromethyl)phenyl)-5-(4-((4-methylpiperazin-1-yl)methyl)phenyl)-7-(tetrahydro-2H-pyran-4-yl)-7H-pyrrolo[2,3-d]pyrimidin-2-amine

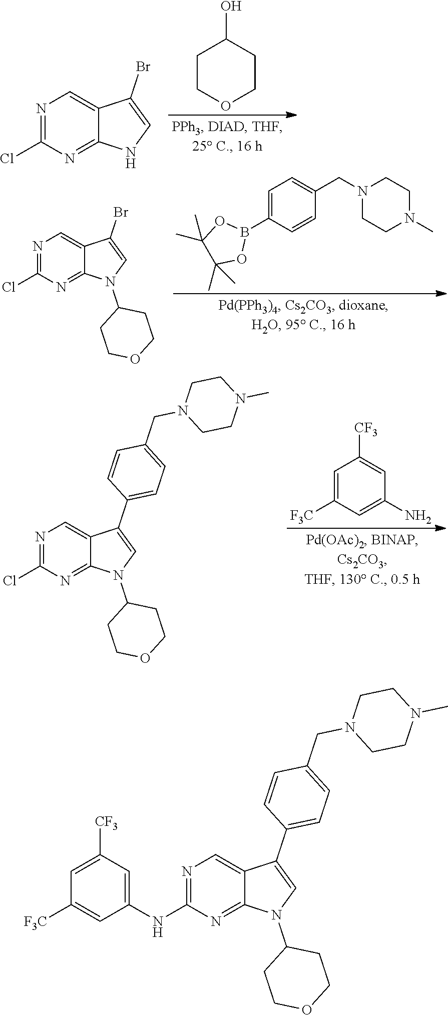

To a solution of 5-bromo-2-chloro-7H-pyrrolo[2,3-d]pyrimidine (1.00 g, 4.30 mmol), triphenylphosphine (1.40 g, 5.20 mmol) and tetrahydro-2H-pyran-4-ol (0.70 g, 6.50 mmol) in dry THF (20 mL) was added diisopropyl (E)-diazene-1,2-dicarboxylate (1.10 g, 5.20 mmol) at 0° C. The reaction mixture was stirred at 0° C. for 0.5 h, then warmed to rt and stirred for 16 h. The reaction mixture was quenched with water (10 mL) and extracted with EtOAc (30 mL, 2×). The combined organic layers were washed with brine (15 mL), dried (Na₂SO₄), and concentrated under reduced pressure. The residue was purified by an ISCO silica gel column (petroleum ether/ethyl acetate=10/1 to 2/1) to afford the desired product (0.92 g, 68%) as a white solid. ¹H NMR (400 MHz, CDCl₃) δ 8.75 (s, 1H), 7.32 (s, 1H), 4.99 (tt, J=11.4, 5.3 Hz, 1H), 4.14 (ddd, J=11.4, 4.3, 2.8 Hz, 2H), 3.69-3.58 (m, 2H), 2.09-1.98 (m, 4H). MS (ESI) m/z calcd for C₁₁H₁₂BrClN₃O: 15.98 [M+H]⁺; found 316.10. LC-MS: >95% purity.

To a solution of 5-bromo-2-chloro-7-(tetrahydro-2H-pyran-4-yl)-7H-pyrrolo[2,3-d]pyrimidine (0.40 g, 1.30 mmol) in 1,4-dioxane (10 mL) and water (1.0 mL) was added 1-methyl-4-(4-(4,4,5,5-tetramethyl-1,3,2-dioxaborolan-2-yl)benzyl) piperazine (0.50 g, 1.50 mmol), cesium carbonate (1.30 g, 3.80 mmol), and Pd(PPh₃)₄ (0.15 g, 0.10 mmol). The reaction mixture was heated under nitrogen atmosphere at 95° C. for 16 h., then cooled to rt, quenched with H₂O, and extracted with EtOAc (20 mL, 2×). The combined organic layers were washed with brine, dried (Na₂SO₄), and concentrated under reduced pressure. The residue was purified by an ISCO reversed column (methanol/water+0.1% HCl, 10-100%) to afford 2-chloro-5-(4-((4-methylpiperazin-1-yl)methyl)phenyl)-7-(tetrahydro-2H-pyran-4-yl)-7H-pyrrolo[2,3-d]pyrimidine (0.25 g, 46%) as a white solid. MS (ESI) m/z calcd for C₂₃H₂₉ClN₅O: 426.20 [M+H]⁺; found 426.20. LC-MS: >95% purity.

To a solution of 2-chloro-5-(4-((4-methylpiperazin-1-yl)methyl)phenyl)-7-(tetrahydro-2H-pyran-4-yl)-7H-pyrrolo[2,3-d]pyrimidine (80 mg, 0.20 mmol) and 3,5-dichloroaniline (128 mg, 0.60 mmol) in dry THF (15 mL) was added palladium (II) acetate (2.0 mg, 9.5 μmol), Sphos (3.0 mg, 9.5 μmol), and Cs₂CO₃ (186 mg, 0.60 mmol). The reaction mixture was heated under nitrogen atmosphere and microwave radiation at 130° C. for 0.5 h, then quenched with water and extracted with EtOAc (20 mL, 3×). The combined organic layers were washed with water and brine, dried (Na₂SO₄), and concentrated. The residue was purified by an HPLC (Acetonitrile/water+0.05% TFA, 8-100%) to afford a slight yellow solid, which was converted to HCl salt the treatment of a 4.0 N HCl solution in 1,4-dioxane followed by lyophilization to provide the desired compound N-(3,5-bis(trifluoromethyl)phenyl)-5-(4-((4-methylpiperazin-1-yl)methyl)phenyl)-7-(tetrahydro-2H-pyran-4-yl)-7H-pyrrolo[2,3-d]pyrimidin-2-amine (35 mg, 31%). ¹H NMR (850 MHz, CD₃OD) δ 9.14 (s, 1H), 8.45 (d, J=1.5 Hz, 2H), 8.11 (s, 1H), 7.87 (d, J=7.9 Hz, 2H), 7.75 (d, J=7.7 Hz, 2H), 7.72 (s, 1H), 4.93-4.87 (m, 2H), 4.50 (s, 2H), 4.16 (dd, J=11.7, 4.4 Hz, 2H), 3.84-3.46 (m, 10H), 3.02 (s, 3H), 2.31-2.24 (m, 2H), 2.10-2.03 (m, 2H). MS (ESI) m/z calcd for C₃₁H₃₃F₆N₆O: 619.25 [M+H]⁺; found 619.20.

Table 10 describes compounds prepared following procedures described in Example 9 using appropriate reagents. (Note: Mer IC₅₀: ++++ means <10 nM; +++ means between 10-100 nM, ++ means between 100 nM-1 μM; + means between 1-30 μM; − means inactive.)

TABLE 10

| | | TYRO3 | MERTK | AXL | Physical Data |
|---|---|---|---|---|---|
| Compound | Structure | IC₅₀ | IC₅₀ | IC₅₀ | MS m/z (M + 1) or/and ¹H NMR |
| 159 | 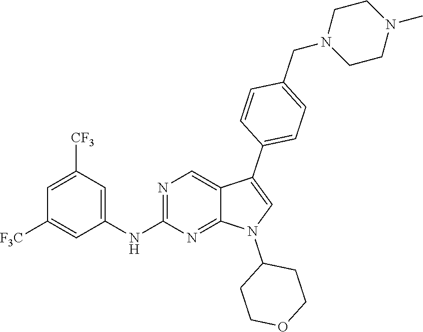 [SMILES: CN1CCN(Cc2ccc(-c3cn(C4CCOCC4)c4nc(Nc5cc(C(F)(F)F)cc(C(F)(F)F)c5)ncc34)cc2)CC1] | ++++ | + | | MS (ESI) m/z calcd for C₃₁H₃₃F₆N₆O: 619.25 [M + H]⁺; found 619.20. ¹H NMR (850 MHz, CD₃OD) δ 9.14 (s, 1H), 8.45 (d, J = 1.5 Hz, 2H), 8.11 (s, 1H), 7.87 (d, J = 7.9 Hz, 2H), 7.75 (d, J = 7.7 Hz, 2H), 7.72 (s, 1H), 4.93-4.87 (m, 2H), 4.50 (s, 2H), 4.16 (dd, J = 11.7, 4.4 Hz, 2H), 3.84-3.46 (m, 10H), 3.02 (s, 3H), 2.31-2.24 (m, 2H), 2.10-2.03 (m, 2H). |
| 160 | 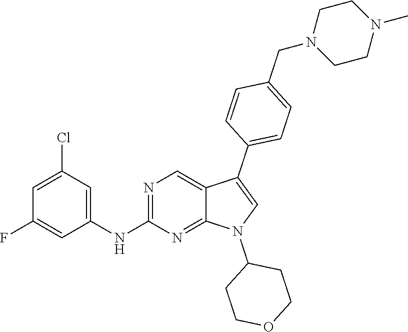 [SMILES: CN1CCN(Cc2ccc(-c3cn(C4CCOCC4)c4nc(Nc5cc(F)cc(Cl)c5)ncc34)cc2)CC1] | ++++ | ++++ | | MS (ESI) m/z calcd for C₂₉H₃₃ClFN₆O: 535.23 [M + H]⁺; found 535.20. ¹H NMR (850 MHz, CD₃OD) δ 9.09 (s, 1H), 8.15 (s, 1H), 7.87 (d, J = 8.2 Hz, 2H), 7.77 (d, J = 8.2 Hz, 2H), 7.75-7.72 (m, 1H), 7.48 (dt, J = 10.4, 2.1 Hz, 1H), 7.07 (dt, J = 8.4, 2.1 Hz, 1H), 4.91-4.87 (m, 2H), 4.53 (s, 2H), 4.19-4.15 (m, 2H), 3.88-3.50 (m, 10H), 3.03 (s, 3H), 2.31 (qd, J = 12.4, 4.5 Hz, 2H), 2.10 (ddd, J = 12.5, 4.2, 1.9 Hz, 2H). |
| 161 | 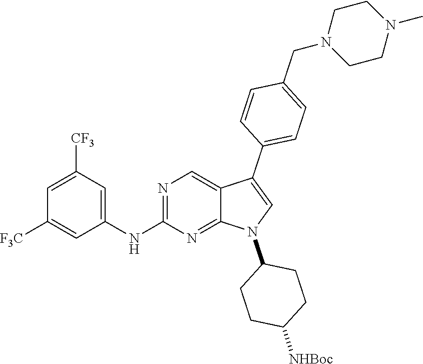 | +++ | | | MS (ESI) m/z calcd for C₃₇H₄₄F₆N₇O₂: 732.34 [M + H]⁺; found 732.30. ¹H NMR (500 MHz, CD₃OD) δ 8.97 (s, 1H), 8.52 (s, 2H), 7.67- 7.62 (m, 3H), 7.44 (s, 1H), 7.39 (d, J = 7.9 Hz, 2H), 4.71-4.62 (m, 2H), 3.56 (s, 2H), 2.54-2.50 (m, 2H), 2.30-2.26 (m, 5H), 2.21-2.11 (m, 6H), 2.10-1.99 (m, 4H), 1.50-1.44 (m, 11H). |
| 162 | 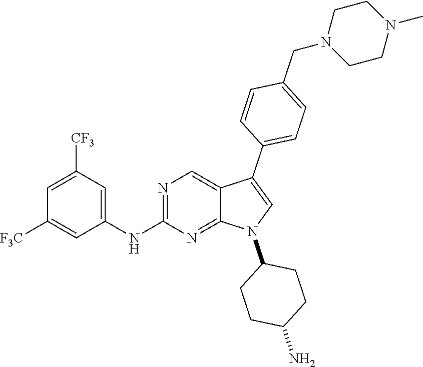 | ++++ | +++ | | MS (ESI) m/z calcd for C₃₅H₃₉F₆N₄: 629.30 [M + H]⁺; found 629.30 ¹H NMR (500 MHz, CD₃OD) δ 9.10 (s, 1H), 8.47 (d, J = 1.6 Hz, 2H), 7.93 (s, 1H), 7.83 (d, J = 8.1 Hz, 2H), 7.69 (d, J = 8.1 Hz, 2H), 7.61 (s, 1H), 4.71-4.66 (m, 1H), 4.36 (s, 2H), 3.73-3.45 (m, 8H), 2.99 (s, 3H), 2.35-2.09 (m, 7H), 1.77-1.65 (m, 2H). |
| 163 | 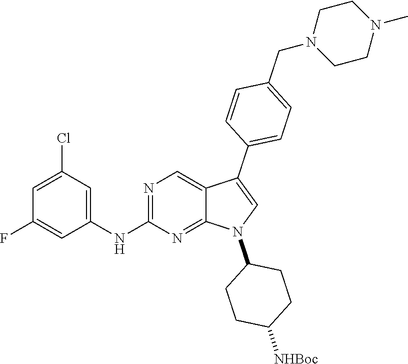 | +++ | ++ | | MS (ESI) m/z calcd for C₃₅H₄₄ClFN₇O₂: 648.32 [M + H]⁺; found 628.30. ¹H NMR (500 MHz, CD₃OD) δ 8.94 (s, 1H), 7.82 (s, 1H), 7.66- 7.56 (m, 4H), 7.39 (d, J = 8.1 Hz, 2H), 6.72 (dt, J = 8.1, 2.1 Hz, 1H), 3.56 (s, 2H), 2.66-2.41 (m, 9H), 2.29 (s, 3H), 2.19-2.11 (m, 4H), 1.56-1.40 (m, 14H). |
| 164 | 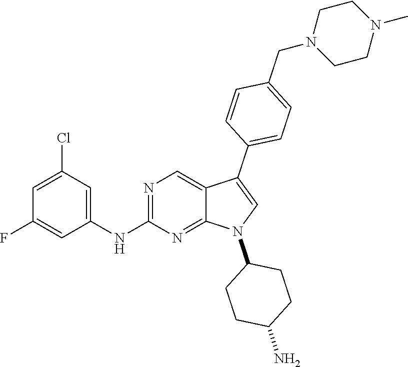 | ++++ | ++++ | | MS (ESI) m/z calcd for C₃₀H₃₆ClFN₇: 548.26 [M + H]⁺; found 548.30. ¹H NMR (500 MHz, CD₃OD) δ 9.10 (s, 1H), 8.47 (d, J = 1.6 Hz, 2H), 7.93 (s, 1H), 7.83 (d, J = 8.1 Hz, 2H), 7.69 (d, J = 7.9 Hz, 2H), 7.61 (s, 1H), 4.71-4.66 (m, 1H), 4.36 (s, 2H), 3.73-3.45 (m, 8H), 2.99 (s, 3H), 2.35-2.09 (m, 7H), 1.77-1.65 (m, 2H). |
| 165 | 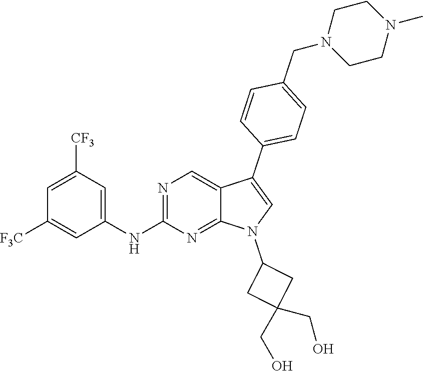 [SMILES: CN1CCN(Cc2ccc(-c3cn(C4CC(CO)(CO)C4)c4nc(Nc5cc(C(F)(F)F)cc(C(F)(F)F)c5)ncc34)cc2)CC1] | ++++ | + | | MS (ESI) m/z calcd for C₃₂H₃₅F₆N₆O₂: 649.27 [M + H]⁺; found 649.30. ¹H NMR (500 MHz, CD₃OD) δ 8.99 (s, 1H), 8.53 (s, 2H), 7.82 (s, 1H), 7.72 (d, J = 8.0 Hz, 2H), 7.48-7.43 (m, 3H), 5.32 (p, J = 8.7 Hz, 1H), 3.78 (s, 2H), 3.76 (s, 2H), 3.64 (s, 3H), 3.29-3.25 (m, 4H), 2.91-2.79 (m, 6H), 2.59- 2.51 (m, 2H), 2.50-2.44 (m, 2H). |

### Example 10—Syntheses trans-/cis-4-(2-((3-Chloro-5-fluorophenyl)amino)-5-(4-((4-methylpiperazin-1-yl)methyl)phenyl)pyrrolo[2,1-f][1,2,4]triazin-7-yl)cyclohexan-1-ol

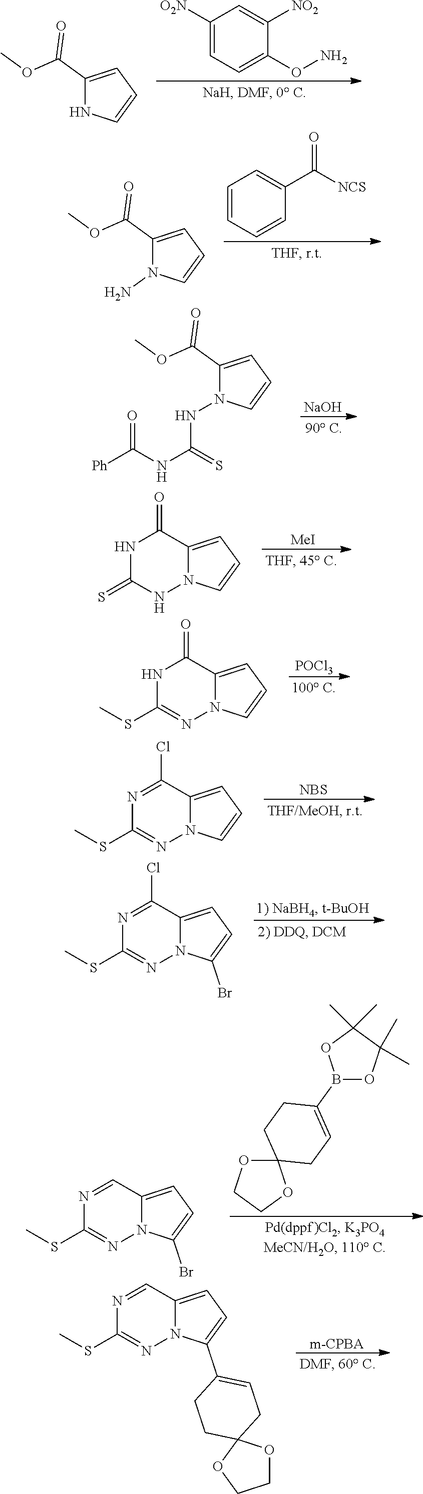

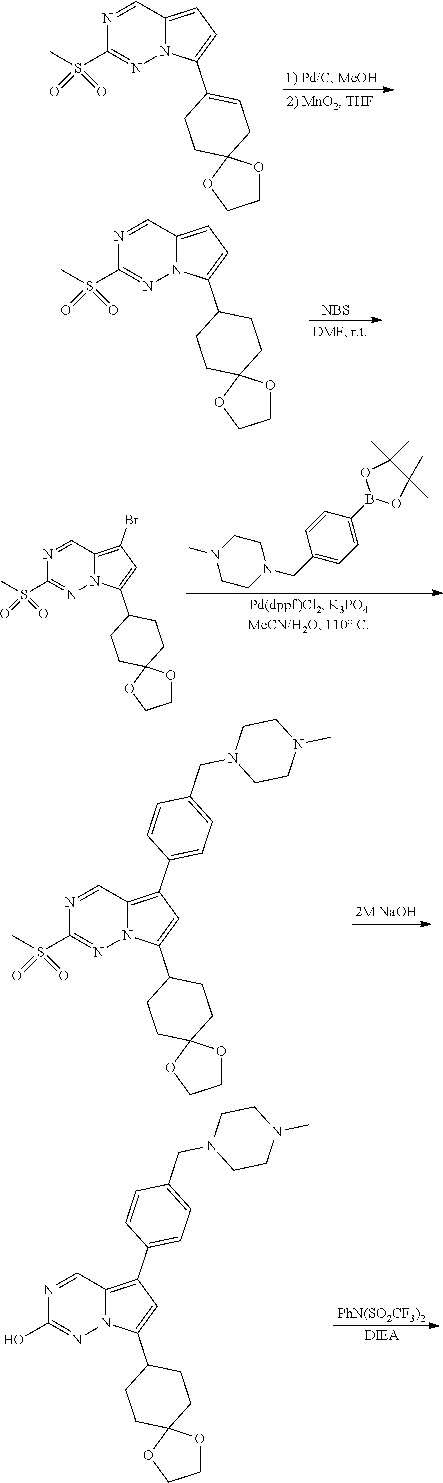

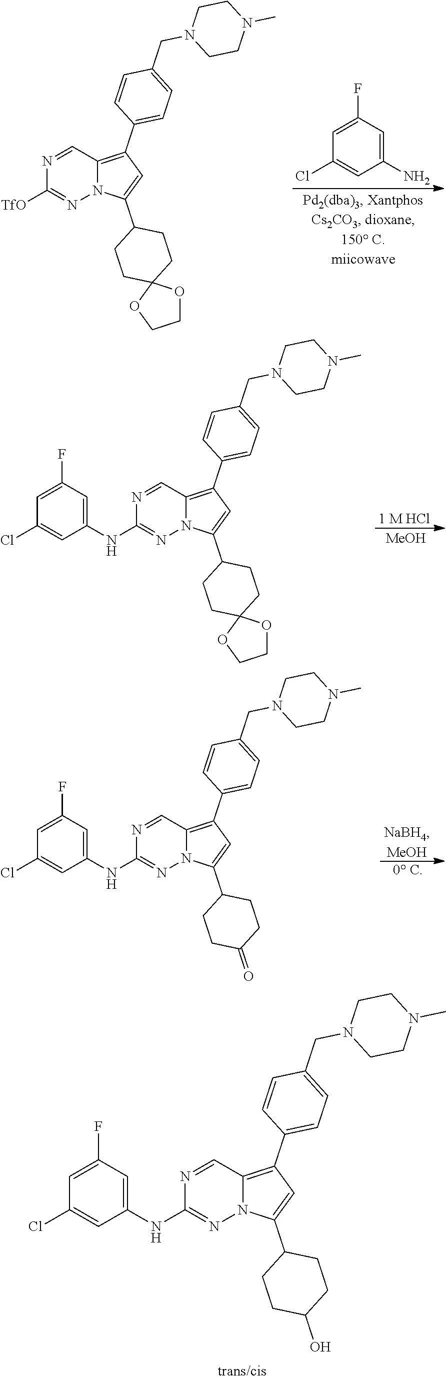

To a solution of methyl 1H-pyrrole-2-carboxylate (5.00 g, 40.0 mmol) in DMF was added NaH (1.92 g, 79.9 mmol) at 0° C. After the reaction mixture was stirred at 0° C. for 1 h, a solution of O-(2,4-dinitrophenyl)hydroxylaminein (11.9 g, 59.9 mmol) in DMF was added dropwise. The mixture was stirred at 0° C. for 3 h, quenched with water and brine, and extracted with EtOAc (3×). The combined organic layers were washed with brine, dried (Na₂SO₄) and concentrated under reduced pressure. The residue was purified by normal phase ISCO to afford methyl 1-amino-1H-pyrrole-2-carboxylate as brown oil. MS (ESI) m/z calcd for C₆H₉N₂O₂: 141.07 [M+H]⁺; found 141.10.

To a solution of methyl 1-amino-1H-pyrrole-2-carboxylate (1.92 g, 13.7 mmol) in THF was added benzoyl isothiocyanate (2.46 g, 15.1 mmol). The reaction mixture was stirred at rt for 12 h and concentrated under reduced pressure. The residue was purified by normal phase ISCO to afford methyl 1-(3-benzoylthioureido)-1H-pyrrole-2-carboxylate as brown solids. MS (ESI) m/z calcd for C₁₄H₁₄N₃O₃S: 304.08 [M+H]⁺; found 304.10. ¹H NMR (400 MHz, Chloroform-d) δ 12.86 (s, 1H), 9.27 (s, 1H), 7.96-7.87 (m, 2H), 7.66 (ddt, J=8.1, 6.9, 1.2 Hz, 1H), 7.60-7.50 (m, 2H), 7.05 (dd, J=2.9, 1.9 Hz, 1H), 7.01 (dd, J=4.3, 1.9 Hz, 1H), 6.27 (dd, J=4.3, 2.9 Hz, 1H), 3.80 (s, 3H).

A suspension of methyl 1-(3-benzoylthioureido)-1H-pyrrole-2-carboxylate (3.36 g, 11.1 mmol) in a 1.0 M aqueous NaOH solution (45 mL). The reaction mixture was stirred at 90° C. for 2 h, then cooled to rt, and purified by reverse phase ISCO to afford 2-thioxo-2,3-dihydropyrrolo[2,1-f][1,2,4]triazin-4 (1H)-one as light brown solids. MS (ESI) m/z calcd for C₆H₆N₃OS: 168.02 [M+H]⁺; found 168.00. ¹H NMR (400 MHz, DMSO-d₆) δ 10.81 (s, 1H), 7.24 (dd, J=2.5, 1.6 Hz, 1H), 6.67 (dd, J=4.2, 1.6 Hz, 1H), 6.32 (dd, J=4.2, 2.5 Hz, 1H), 4.12 (br, 1H).

A suspension of 2-thioxo-2,3-dihydropyrrolo[2,1-f][1,2,4]triazin-4 (1H)-one (1.90 g, 11.4 mmol) in anhydrous THF (30 mL) was added a solution of methyl iodide (2.10 mg, 14.8 mmol) in THF (5.0 mL) dropwise. The reaction mixture was stirred at 45° C. for 1 h and concentrated under reduced pressure. The residue was purified by normal phase ISCO to afford 2-(methylthio)pyrrolo[2,1-f][1,2,4]triazin-4(3H)-one as off-white solids. MS (ESI) m/z calcd for C₇H₈N₃OS: 182.04 [M+H]⁺; found 182.20. ¹H NMR (400 MHz, DMSO-d₆) δ 12.02 (s, 1H), 7.52 (dd, J=2.6, 1.7 Hz, 1H), 6.84 (dd, J=4.3, 1.7 Hz, 1H), 6.47 (dd, J=4.3, 2.6 Hz, 1H), 2.52 (s, 3H).

A suspension of 2-(methylthio)pyrrolo[2,1-f][1,2,4]triazin-4(3H)-one (2.50 g, 13.8 mmol) in phosphorus oxychloride (20 mL) was heated at 100° C. for 5 h, then cooled to room. Excess POCl₃ was removed under reduced pressure. The residue was suspended in saturated aqueous Na₂CO₃ and extracted with EtOAc (3×). The combined organic layers were washed with brine, dried (Na₂SO₄), and concentrated under reduced pressure. The residue was purified by normal phase ISCO to afford 4-chloro-2-(methylthio)pyrrolo[2,1-f][1,2,4]triazine as solids. MS (ESI) m/z calcd for C₇H₇ClN₃S: 200.00 [M+H]⁺; found 200.00. ¹H NMR (400 MHz, Chloroform-d) δ 7.73 (dd, J=2.5, 1.5 Hz, 1H), 6.90 (dd, J=4.6, 1.5 Hz, 1H), 6.80 (dd, J=4.6, 2.5 Hz, 1H), 2.57 (s, 3H).

To a solution of 4-chloro-2-(methylthio)pyrrolo[2,1-f][1,2,4]triazine (1.68 g, 8.43 mmol) in a mixture of THF (30 mL) and MeOH (20 mL) was added NBS (1.50 g, 8.43 mmol) slowly at rt. The reaction mixture was stirred at rt for 2 h and concentrated under reduced pressure. The residue was suspended in water and extracted with DCM (2×). The combined organic layers were washed with brine, dried (Na₂SO₄), and concentrated under reduced pressure to afford 7-bromo-4-chloro-2-(methylthio)pyrrolo[2,1-f][1,2,4]triazine as off-white solids that were used in the next step without further purification. MS (ESI) m/z calcd for C₇H₇ClN₃S: 277.92 [M+H]⁺; found 278.10.

To a solution of 7-bromo-4-chloro-2-(methylthio)pyrrolo[2,1-f][1,2,4]triazine (2.67 g, 9.60 mmol) in t-BuOH was added sodium borohydride (726 mg, 19.2 mmol). The reaction mixture was stirred at 60° C. for 5 h. After cooling to rt, the mixture was filtered through celite. Solvents were removed under reduced pressure. The residue was suspended in DCM and was added DDQ (2.40 g, 10.6 mmol). The reaction mixture was stirred at rt for 30 min. The slurry was filtered through celite and washed with DCM. The solvent was removed under reduced pressure. The residue was purified by normal phase ISCO to afford 7-bromo-2-(methylthio)pyrrolo[2,1-f][1,2,4]triazine as white solids. MS (ESI) m/z calcd for C₇H₇BrN₃S: 243.95 [M+H]⁺; found 244.10. ¹H NMR (400 MHz, DMSO-d₆) δ 8.95 (s, 1H), 7.27-6.60 (m, 2H), 2.57 (s, 3H).

To a solution of 7-bromo-2-(methylthio)pyrrolo[2,1-f][1,2,4]triazine (1.41 g, 5.78 mmol), 4,4,5,5-tetramethyl-2-(1,4-dioxaspiro[4.5]dec-7-en-8-yl)-1,3,2-dioxaborolane (1.84 g, 6.93 mmol) and K₃PO₄ (3.68 g, 17.3 mmol) in a mixture of acetonitrile/water (36 mL/4 mL) was added 1,1′-bis(diphenylphosphino) ferrocene-palladium (II) dichloride (295.8 mg, 404.3 μmol). The reaction mixture was degassed and stirred under nitrogen atmosphere at 110° C. for 12 h. After cooling to rt, the mixture was filtered and washed with EtOAc. Solvents were removed under reduced pressure. The residue was purified by normal phase ISCO to afford 2-(methylthio)-7-(1,4-dioxaspiro[4.5]dec-7-en-8-yl)pyrrolo[2,1-f][1,2,4]triazine as yellow solids. MS (ESI) m/z calcd for C₁₅H₁₈N₃O₂S: 304.11 [M+H]⁺; found 304.10. ¹H NMR (400 MHz, Chloroform-d) δ 8.65 (s, 1H), 7.16-7.03 (m, 1H), 6.82 (d, J=4.8 Hz, 1H), 6.77 (d, J=4.8 Hz, 1H), 4.03 (s, 4H), 2.90-2.77 (m, 2H), 2.60-2.57 (m, 2H), 2.57 (s, 3H), 1.96 (tt, J=6.6, 0.9 Hz, 2H).

To a solution of 2-(methylthio)-7-(1,4-dioxaspiro[4.5]dec-7-en-8-yl)pyrrolo[2,1-f][1,2,4]triazine (830 mg, 2.74 mmol) in DMF was added m-CPBA (1.42 g, 8.21 mmol). The reaction mixture was stirred at 60° C. for 12 h, then quenched with saturated aqueous Na₂CO₃. After stirring for 30 min, the mixture was extracted with EtOAc (3×). The combined organic layers were washed with brine, dried (Na₂SO₄), and concentrated under reduced pressure. The residue was purified by normal phase ISCO to afford 2-(methylthio)-7-(1,4-dioxaspiro[4.5]dec-7-en-8-yl)pyrrolo[2,1-f][1,2,4]triazine as yellow solids. MS (ESI) m/z calcd for C₁₅H₁₈N₃O₄S: 336.10 [M+H]⁺; found 336.20. ¹H NMR (400 MHz, Chloroform-d) δ 8.90 (s, 1H), 7.21 (tt, J=4.3, 1.6 Hz, 1H), 7.07 (d, J=4.8 Hz, 1H), 6.99 (d, J=4.8 Hz, 1H), 4.01 (s, 4H), 2.97 (s, 3H), 2.87-2.79 (m, 2H), 2.63-2.54 (m, 2H), 1.95 (t, J=6.6 Hz, 2H).

To a solution of 2-(methylsulfonyl)-7-(1,4-dioxaspiro[4.5]dec-7-en-8-yl)pyrrolo[2,1-f][1,2,4]triazine (1.0 g, 3.0 mmol) in a mixture of THF (20 mL) and MeOH (30 mL) was added Pd/C (100 mg). The reaction mixture was stirred under hydrogen atmosphere at rt overnight, then filtered through celite and washed with a mixture of DCM/MeOH. Solvents were removed under reduced pressure. The residue was redissolved in THF and was added activated manganese dioxide (1.30 g, 15 mmol). The reaction mixture was stirred at 50° C. for 2 h, cooled to rt, and filtered through celite. The filtrate was concentrated under reduced pressure. The residue was purified by normal phase ISCO to afford 2-(methylsulfonyl)-7-(1,4-dioxaspiro[4.5]decan-8-yl)pyrrolo[2,1-f][1,2,4]triazine as yellow solids. MS (ESI) m/z calcd for C₁₅H₂₀N₃O₄S: 338.12 [M+H]⁺; found 338.30. ¹H NMR (400 MHz, Chloroform-d) δ 8.95 (s, 1H), 7.06 (d, J=4.8 Hz, 1H), 7.04 (d, J=4.8 Hz, 1H), 4.06-3.92 (m, 4H), 3.45-3.33 (m, 4H), 2.16-2.06 (m, 2H), 1.96-1.70 (m, 6H).

To a solution of 2-(methylsulfonyl)-7-(1,4-dioxaspiro[4.5]decan-8-yl)pyrrolo[2,1-f][1,2,4]triazine (1.0 g, 3.0 mmol) in DMF was added NBS (580 mg, 3.3 mmol). The reaction mixture was stirred at rt for 24 h, quenched with water, was extracted with EtOAc (3×). The combined organic layers were washed with brine, dried (Na₂SO₄), and concentrated under reduced pressure. The residue was further purified by normal phase ISCO to afford 5-bromo-2-(methylsulfonyl)-7-(1,4-dioxaspiro[4.5]decan-8-yl)pyrrolo[2,1-f][1,2,4]triazine as yellow solids. MS (ESI) m/z calcd for C₁₅H₂₀N₃O₄S: 415.02 [M+H]⁺; found 415.20.

To a solution of 5-bromo-2-(methylsulfonyl)-7-(1,4-dioxaspiro[4.5]decan-8-yl)pyrrolo[2,1-f][1,2,4]triazine (333 mg, 800 μmol), 1-methyl-4-(4-(4,4,5,5-tetramethyl-1,3,2-dioxaborolan-2-yl)benzyl) piperazine (278 mg, 880 mmol), and K₃PO₄ (509 mg, 2.40 mmol) in a mixture of acetonitrile (27 mL) and water (3 mL) was added 1,1′-bis(diphenylphosphino) ferrocene-palladium (II) dichloride (58.5 mg, 80.0 μmol). The reaction mixture was degassed and stirred under nitrogen atmosphere at 110° C. for 3 h.

After cooling to rt, the mixture was filtered and washed with EtOAc. Solvents were removed under reduced pressure. The residue was purified by normal phase ISCO to afford 5-(4-((4-methylpiperazin-1-yl)methyl)phenyl)-2-(methylsulfonyl)-7-(1,4-dioxaspiro[4.5]decan-8-yl)pyrrolo[2,1-f][1,2,4]triazine as yellow solids. MS (ESI) m/z calcd for C₁₅H₂₀N₃O₄S: 525.24 [M+H]⁺; found 525.20. ¹H NMR (400 MHz, Methanol-d₄) δ 9.23 (s, 1H), 7.67 (d, J=8.1 Hz, 2H), 7.47 (d, J=8.1 Hz, 2H), 7.34 (s, 1H), 3.98 (s, 4H), 3.60 (s, 2H), 3.46-3.36 (m, 4H), 2.54 (br, 8H), 2.29 (s, 3H), 2.22-2.07 (m, 2H), 2.04-1.85 (m, 4H), 1.84-1.72 (m, 2H).

A suspension of 5-(4-((4-methylpiperazin-1-yl)methyl)phenyl)-2-(methylsulfonyl)-7-(1,4-dioxaspiro[4.5]decan-8-yl)pyrrolo[2,1-f][1,2,4]triazine (330 mg, 628 μmol) in a 2.0 M aqueous NaOH solution (15 mL) was stirred at 70° C. for 12 h and then quenched with MeOH (5.0 mL). The mixture was purified by reverse phase ISCO to afford 5-(4-((4-methylpiperazin-1-yl)methyl)phenyl)-7-(1,4-dioxaspiro[4.5]decan-8-yl)pyrrolo[2,1-f][1,2,4]triazin-2-ol as yellow solids. MS (ESI) m/z calcd for C₁₅H₂₀N₃O₄S: 463.26 [M+H]⁺; found 463.30. ¹H NMR (400 MHz, Methanol-d₄) δ 8.73 (s, 1H), 7.53 (d, J=7.9 Hz, 2H), 7.36 (d, J=7.9 Hz, 2H), 6.57 (s, 1H), 3.96 (s, 4H), 3.55 (s, 2H), 3.29-3.20 (m, 1H), 2.52 (br, 8H), 2.28 (s, 3H), 2.19-2.08 (m, 2H), 1.89-1.80 (m, 2H), 1.79-1.67 (m, 4H).

To a solution of DIEA (328 μL, 1.88 mmol) and 5-(4-((4-methylpiperazin-1-yl)methyl)phenyl)-7-(1,4-dioxaspiro[4.5]decan-8-yl)pyrrolo[2,1-f][1,2,4]triazin-2-ol (291 mg, 628 μmol) in methoxyl propanol was added phenyl triflimide (673 mg, 1.88 mmol). The reaction mixture was stirred at rt for 24 h, quenched with water, and extracted with EtOAc (3×). The combined organic layers were washed with brine, dried (Na₂SO₄), and concentrated under reduced pressure. The residue was purified by normal phase ISCO to afford 5-(4-((4-methylpiperazin-1-yl)methyl)phenyl)-7-(1,4-dioxaspiro[4.5]decan-8-yl)pyrrolo[2,1-f][1,2,4]triazin-2-yl trifluoromethanesulfonate as yellow solids. MS (ESI) m/z calcd for C₂₇H₃₃F₃N₅O₅S: 596.22 [M+H]⁺; found 596.20.

To a suspension of cesium carbonate (109 mg, 336 μmol), 5-(4-((4-methylpiperazin-1-yl)methyl)phenyl)-7-(1,4-dioxaspiro[4.5]decan-8-yl)pyrrolo[2,1-f][1,2,4]triazin-2-yl trifluoromethanesulfonate (100 mg, 168 μmol) and 3-chloro-5-fluoroaniline (73.3 mg, 504 μmol) in dioxane (10 mL) was added tris(dibezylideneacetone)dipalladium (15.4 mg, 16.8 μmol) and 4,5-bis(diphenylphosphino)-9,9-dimethyl xanthene (19.4 mg, 33.6 μmol). The reaction mixture was degassed and stirred under nitrogen atmosphere and microwave radiation at 150° C. for 20 min. After cooling to rt, the reaction was filtered through celite and washed with a mixture of DCM and MeOH. The filtrate was concentrated under reduced pressure. The residue was purified by normal phase ISCO to afford N-(3-chloro-5-fluorophenyl)-5-(4-((4-methylpiperazin-1-yl)methyl)phenyl)-7-(1,4-dioxaspiro[4.5]decan-8-yl)pyrrolo[2,1-f][1,2,4]triazin-2-amine as yellow solids. MS (ESI) m/z calcd for C₃₂H₃₆ClFN₆O₂: 590.26 [M+H]⁺; found 590.20. ¹H NMR (400 MHz, DMSO-d₆) δ 9.14 (s, 1H), 7.86 (t, J=2.2 Hz, 2H), 7.67 (d, J=8.2 Hz, 2H), 7.63 (dt, J=11.9, 2.2 Hz, 1H), 7.36 (d, J=8.2 Hz, 2H), 6.98 (s, 1H), 6.96 (dt, J=8.4, 2.2 Hz, 2H), 3.92 (s, 4H), 3.48 (s, 2H), 3.23-3.14 (m, 1H), 2.46-2.23 (m, 8H), 2.21-2.12 (m, 5H), 1.90-1.82 (m, 2H), 1.80-1.68 (m, 4H).

To a solution of N-(3-chloro-5-fluorophenyl)-5-(4-((4-methylpiperazin-1-yl)methyl)phenyl)-7-(1,4-dioxaspiro[4.5]decan-8-yl)pyrrolo[2,1-f][1,2,4]triazin-2-amine (80.0 mg, 135 μmol) in a mixture of MeCN (5.0 mL) and MeOH (5.0 mL) was added a 1.0 M aqueous HCl solution (1.0 mL). The reaction mixture was stirred at rt for 3 h, quenched with saturated aqueous Na₂CO₃, and extracted with EtOAc (3×). The combined organic layers were washed with brine, dried (Na₂SO₄), and concentrated under reduced pressure. The residue was purified by reverse phase ISCO to afford 4-(2-((3-chloro-5-fluorophenyl)amino)-5-(4-((4-methylpiperazin-1-yl)methyl)phenyl)pyrrolo[2,1-f][1,2,4]triazin-7-yl)cyclohexan-1-one as yellow solids. MS (ESI) m/z calcd for C₃₀H₃₂ClFN₆O: 547.24 [M+H]⁺; found 547.30. ¹H NMR (400 MHz, DMSO-d₆) δ 10.06 (s, 1H), 9.19 (s, 1H), 7.87 (t, J=2.1 Hz, 1H), 7.78 (d, J=7.9 Hz, 2H), 7.63 (dt, J=11.9, 2.1 Hz, 1H), 7.51 (d, J=7.9 Hz, 2H), 7.05 (s, 1H), 6.96 (dt, J=8.5, 2.1 Hz, 1H), 4.08 (s, 2H), 3.64 (tt, J=11.8, 3.3 Hz, 1H), 3.21 (br, 8H), 2.83 (s, 3H), 2.78-2.61 (m, 2H), 2.58-2.33 (m, 4H), 1.99 (qd, J=13.1, 4.0 Hz, 2H).

To a solution of 4-(2-((3-chloro-5-fluorophenyl)amino)-5-(4-((4-methylpiperazin-1-yl)methyl)phenyl)pyrrolo[2,1-f][1,2,4]triazin-7-yl)cyclohexan-1-one (65.0 mg, 119 μmol) in methanol was added NaBH₄ (13.5 mg, 356 μmol). The reaction mixture was stirred at rt for 1.5 h, quenched with water, and extracted with EtOAc (3×). The combined organic layers were washed with brine, dried (Na₂SO₄), and concentrated under reduced pressure. The residue was purified by preparative HPLC to afford the trans- and cis-isomers respectively. trans-4-(2-((3-chloro-5-fluorophenyl)amino)-5-(4-((4-methylpiperazin-1-yl)methyl)phenyl)pyrrolo[2,1-f][1,2,4]triazin-7-yl)cyclohexan-1-ol. ¹H NMR (400 MHz, DMSO-d₆) δ 10.01 (s, 1H), 9.14 (s, 1H), 7.85 (t, J=2.1 Hz, 1H), 7.77 (d, J=8.1 Hz, 2H), 7.63 (dt, J=11.9, 2.1 Hz, 1H), 7.51 (d, J=8.1 Hz, 2H), 6.98 (s, 1H), 6.95 (dt, J=8.5, 2.1 Hz, 1H), 4.13 (s, 2H), 3.53 (tt, J=10.5, 4.1 Hz, 1H), 3.24 (br, 8H), 3.05 (tt, J=11.8, 3.4 Hz, 1H), 2.84 (s, 3H), 2.24-2.10 (m, 2H), 2.07 (s, 1H), 2.05-1.92 (m, 2H), 1.65-1.51 (m, 2H), 1.49-1.33 (m, 2H). MS (ESI) m/z calcd for C₃₀H₃₅ClFN₆O: 549.25 [M+H]⁺; found 549.20. cis-4-(2-((3-chloro-5-fluorophenyl)amino)-5-(4-((4-methylpiperazin-1-yl)methyl)phenyl)pyrrolo[2,1-f][1,2,4]triazin-7-yl)cyclohexan-1-ol. ¹H NMR (400 MHz, DMSO-d₆) δ 10.00 (s, 1H), 9.15 (s, 1H), 7.86 (t, J=2.2 Hz, 1H), 7.77 (d, J=7.9 Hz, 2H), 7.64 (dt, J=12.0, 2.2 Hz, 1H), 7.46 (d, J=7.9 Hz, 2H), 7.01 (s, 1H), 6.96 (dt, J=8.5, 2.2 Hz, 1H), 4.51 (br, 8H), 4.02-3.95 (m, 1H), 3.89 (s, 2H), 3.22-3.04 (m, 1H), 2.80 (s, 3H), 2.03-1.87 (m, 4H), 1.86-1.77 (m, 2H), 1.75-1.63 (m, 2H). MS (ESI) m/z calcd for C₃₀H₃₅ClFN₆O: 549.25 [M+H]⁺; found 549.20.

Table 11 describes compounds prepared following procedures described in Example 10 using appropriate reagents. (Note: Mer IC₅₀: ++++ means <10 nM; + means between 10-100 nM, ++ means between 100 nM-1 μM; + means between 1-30 μM; − means inactive.).

TABLE 11

| | | TYRO3 | MERTK | AXL | Physical Data |
|---|---|---|---|---|---|
| Compound | Structure | IC₅₀ | IC₅₀ | IC₅₀ | MS m/z (M + 1) or/and ¹H NMR |
| 71 | 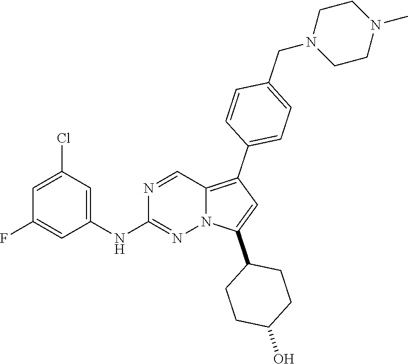 | ++++ | ++++ | | ¹H NMR (400 MHz, DMSO-d₆) δ 10.01 (s, 1H), 9.14 (s, 1H), 7.85 (t, J = 2.1 Hz, 1H), 7.77 (d, J = 8.1 Hz, 2H), 7.63 (dt, J = 11.9, 2.1 Hz, 1H), 7.51 (d, J = 8.1 Hz, 2H), 6.98 (s, 1H), 6.95 (dt, J = 8.5, 2.1 Hz, 1H), 4.13 (s, 2H), 3.53 (tt, J = 10.5, 4.1 Hz, 1H), 3.24 (br, 8H), 3.05 (tt, J = 11.8, 3.4 Hz, 1H), 2.84 (s, 3H), 2.24-2.10 (m, 2H), 2.07 (s, 1H), 2.05-1.92 (m, 2H), 1.65-1.51 (m, 2H), 1.49-1.33 (m, 2H). MS (ESI) m/z calcd for C₃₀H₃₅ClFN₆O: 549.25 [M + H]⁺; found 549.20. |
| 73 | 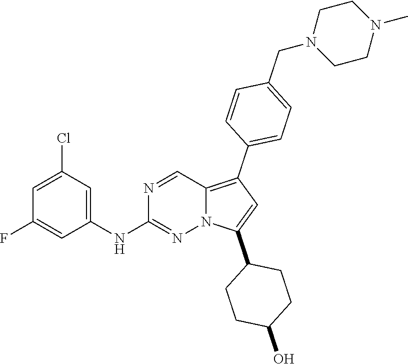 | ++++ | +++ | | ¹H NMR (400 MHz, DMSO-d₆) δ 10.00 (s, 1H), 9.15 (s, 1H), 7.86 (t, J = 2.2 Hz, 1H), 7.77 (d, J = 7.9 Hz, 2H), 7.64 (dt, J = 12.0, 2.2 Hz, 1H), 7.46 (d, J = 7.9 Hz, 2H), 7.01 (s, 1H), 6.96 (dt, J = 8.5, 2.2 Hz, 1H), 4.51 (br, 8H), 4.02-3.95 (m, 1H), 3.89 (s, 2H), 3.22-3.04 (m, 1H), 2.80 (s, 3H), 2.03-1.87 (m, 4H), 1.86-1.77 (m, 2H), 1.75-1.63 (m, 2H). MS (ESI) m/z calcd for C₃₀H₃₅ClFN₆O: 549.25 [M + H]⁺; found 549.20. |
| 72 | 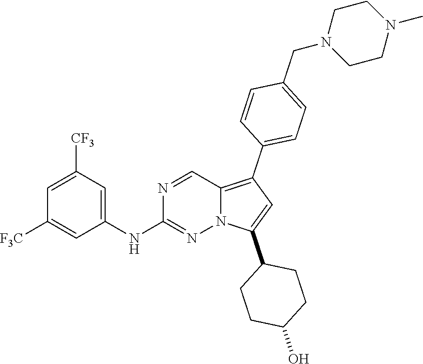 | ++++ | + | | ¹H NMR (400 MHz, DMSO-d₆) δ 10.29 (s, 1H), 9.17 (s, 1H), 8.51 (s, 2H), 7.77 (d, J = 8.1 Hz, 2H), 7.61 (s, 1H), 7.48 (d, J = 8.1 Hz, 2H), 7.03 (s, 1H), 5.12 (br, 8H), 3.96 (s, 2H), 3.57- 3.47 (m, 1H), 3.18-3.03 (m, 1H), 2.82 (s, 3H), 2.16-2.05 (m, 2H), 2.05-1.94 (m, 2H), 1.65-1.49 (m, 2H), 1.47-1.29 (m, 2H). MS (ESI) m/z calcd for C₃₂H₃₅F₆N₆O: 633.28 [M + H]⁺; found 633.40. |
| 74 | 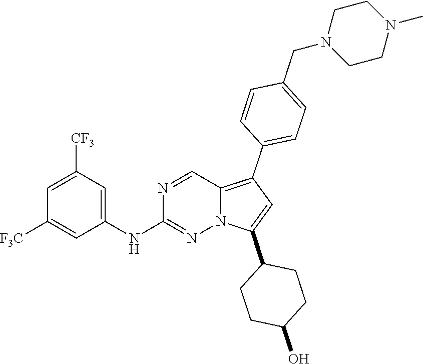 | +++ | + | | ¹H NMR (400 MHz, DMSO-d₆) δ 10.28 (s, 1H), 9.17 (s, 1H), 8.51 (s, 2H), 7.76 (d, J = 7.8 Hz, 2H), 7.61 (s, 1H), 7.43 (d, J = 7.8 Hz, 2H), 7.04 (s, 1H), 4.00-3.94 (m, 1H), 3.72 (s, 2H), 3.66-3.29 (m, 8H), 3.27-3.17 (m, 1H), 2.77 (s, 3H), 2.05-1.75 (m, 6H), 1.67-1.56 (m, 2H). MS (ESI) m/z calcd for C₃₂H₃₅F₆N₆O: 633.28 [M + H]⁺; found 633.30. |
| 170 | 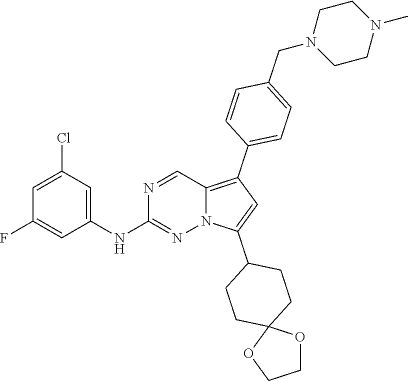 [SMILES: CN1CCN(Cc2ccc(-c3cc(C4CCC5(CC4)OCCO5)n4nc(Nc5cc(F)cc(Cl)c5)ncc34)cc2)CC1] | ++++ | +++ | | ¹H NMR (400 MHz, DMSO-d₆) δ 9.14 (s, 1H), 7.86 (t, J = 2.2 Hz, 2H), 7.67 (d, J = 8.2 Hz, 2H), 7.63 (dt, J = 11.9, 2.2 Hz, 1H), 7.36 (d, J = 8.2 Hz, 2H), 6.98 (s, 1H), 6.96 (dt, J = 8.4, 2.2 Hz, 2H), 3.92 (s, 4H), 3.48 (s, 2H), 3.23-3.14 (m, 1H), 2.46-2.23 (m, 8H), 2.21-2.12 (m, 5H), 1.90- 1.82 (m, 2H), 1.80-1.68 (m, 4H). MS (ESI) m/z calcd for C₃₂H₃₇ClFN₆O₂: 591.27 [M + H]⁺; found 591.20. |
| 171 | 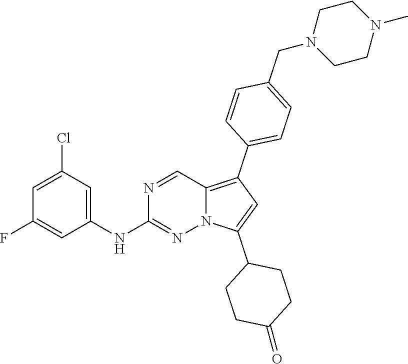 [SMILES: CN1CCN(Cc2ccc(-c3cc(C4CCC(=O)CC4)n4nc(Nc5cc(F)cc(Cl)c5)ncc34)cc2)CC1] | ++++ | +++ | | ¹H NMR (400 MHz, DMSO-d₆) δ 10.06 (s, 1H), 9.19 (s, 1H), 7.87 (t, J = 2.1 Hz, 1H), 7.78 (d, J = 7.9 Hz, 2H), 7.63 (dt, J = 11.9, 2.1 Hz, 1H), 7.51 (d, J = 7.9 Hz, 2H), 7.05 (s, 1H), 6.96 (dt, J = 8.5, 2.1 Hz, 1H), 4.08 (s, 2H), 3.64 (tt, J = 11.8, 3.3 Hz, 1H), 3.21 (br, 8H), 2.83 (s, 3H), 2.78- 2.61 (m, 2H), 2.58-2.33 (m, 4H), 1.99 (qd, J = 13.1, 4.0 Hz, 2H). MS (ESI) m/z calcd for C₃₀H₃₃ClFN₆O: 547.24 [M + H]⁺; found 547.30. |

### Example 11—Syntheses trans-4-(2-((3,5-Bis(trifluoromethyl)phenyl)amino)-5-(2-fluoro-4-((4-methylpiperazin-1-yl)methyl)phenyl)-7H-pyrrolo[2,3-d]pyrimidin-7-yl)cyclohexan-1-ol

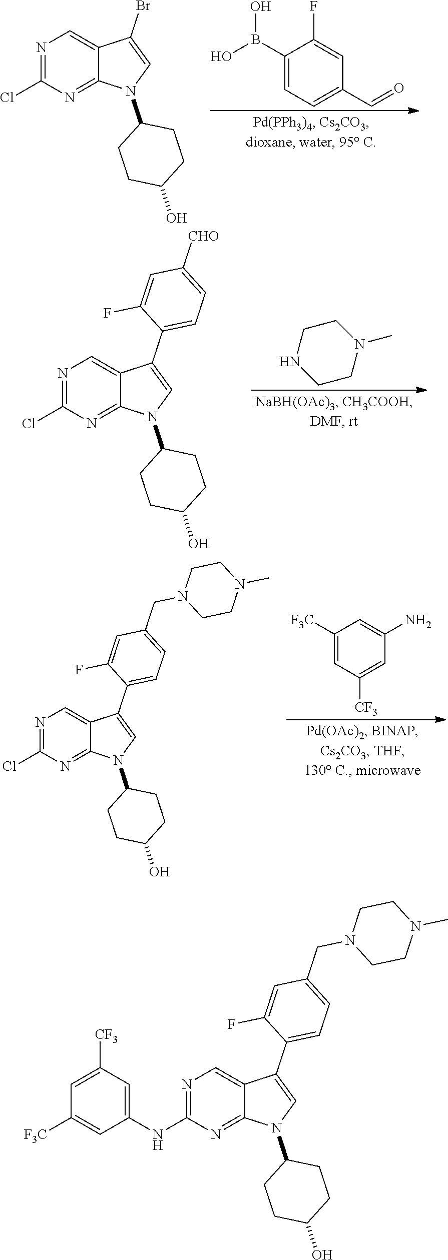

To a solution of trans-4-(5-bromo-2-chloro-7H-pyrrolo[2,3-d]pyrimidin-7-yl)cyclohexan-1-ol (0.40 g, 1.2 mmol) in a mixture of 1,4-dioxane (10 mL) and water (1.0 mL) was added (2-fluoro-4-formylphenyl) boronic acid (0.50 g, 3.0 mmol), cesium carbonate (1.2 g, 3.6 mmol), and Pd(PPh₃)₄ (115 mg, 100 μmol). The reaction mixture was heated under nitrogen atmosphere at 95° C. for 12 h, quenched with H₂O at rt, and extracted with EtOAc (10 mL, 2×). The combined organic layer was washed with brine, dried (Na₂SO₄), and concentrated under reduced pressure. The residue was purified by an ISCO reverse column (acetonitrile/water+0.1% HCl, 10-100%) to afford 4-(2-chloro-7-(trans-4-hydroxycyclohexyl)-7H-pyrrolo[2,3-d]pyrimidin-5-yl)-3-fluorobenzaldehyde (0.20 g, 44%) as a yellow solid. MS (ESI) m/z calcd for C₁₉H₁₈ClFN₃O₂: 374.10 [M+H]⁺; found 374.10. LC-MS: >95% purity.

To a mixture of 4-(2-chloro-7-(trans-4-hydroxycyclohexyl)-7H-pyrrolo[2,3-d]pyrimidin-5-yl)-3-fluorobenzaldehyde (150 mg, 0.40 mmol) in DMF (3.0 mL) was added 1-methylpiperazine (80 mg, 0.80 mmol), acetic acid (48 mg, 0.80 mmol), and sodium triacetoxyborohydride (170 mg, 0.80 mmol). The reaction mixture was stirred at rt for 12 h, quenched with MeOH, and filtrated. The filtrate was concentrated under reduced pressure. The residue was purified by an ISCO silica gel column (hexane/ethyl acetate=2/1 to 1/1) to yield the desired product trans-4-(2-chloro-5-(2-fluoro-4-((4-methylpiperazin-1-yl)methyl)phenyl)-7H-pyrrolo[2,3-d]pyrimidin-7-yl)cyclohexan-1-ol (85 mg, 47%) as a yellow solid. ¹H NMR (400 MHz, CD₃OD) δ 9.08 (s, 1H), 8.02 (s, 1H), 7.58-7.47 (m, 3H), 3.89-3.87 (m, 1H), 3.85 (s, 2H), 3.78-3.68 (m, 1H), 2.96-2.93 (m, 2H), 2.90-2.90 (m, 2H), 2.89 (s, 3H), 2.20-2.11 (m, 4H), 2.09-1.95 (m, 6H), 1.67-1.49 (m, 2H). MS (ESI) for [M+H]⁺ (C₂₄H₃₀ClFN₅O⁺): calcd. m/z 458.20; found m/z 458.20. LC/MS: 95% purity.

To a solution of trans-4-(2-chloro-5-(2-fluoro-4-((4-methylpiperazin-1-yl)methyl)phenyl)-7H-pyrrolo[2,3-d]pyrimidin-7-yl)cyclohexan-1-ol (35 mg, 0.080 mmol) and 3,5-dichloroaniline (80 mg, 0.40 mmol) in dry THF (12 mL) were added palladium (II) acetate (2.0 mg, 8.0 μmol), BINAP (6.0 mg, 8.0 μmol), and Cs₂CO₃ (82 mg, 2.5 mmol). The reaction mixture was heated under nitrogen atmosphere and microwave radiation at 130° C. for 1 h, quenched by water at rt, and extracted with EtOAc (20 mL, 2×). The combined organic layer was washed with water and brine, dried (Na₂SO₄), and concentrated under reduced pressure. The residue was purified by pre-HPLC (Acetonitrile/water+0.05% TFA, 8-100%) to afford a slight yellow solid, which was treated with a 4.0 N solution of HCl in 1,4-dioxane followed by lyophilization to provide HCl salt of the title compound (15 mg, 57%) as a yellow solid. ¹H NMR (500 MHz, CD₃OD) δ 9.15 (s, 1H), 8.44 (s, 2H), 8.15 (s, 1H), 7.80-7.73 (m, 2H), 7.71-7.62 (m, 3H), 4.66 (tt, J=11.7, 4.1 Hz, 1H), 4.42 (s, 2H), 3.78-3.68 (m, 3H), 3.68-3.47 (m, 6H), 3.00 (s, 3H), 2.20-2.10 (m, 4H), 2.10-2.02 (m, 2H), 1.54 (qd, J=12.9, 4.0 Hz, 2H). MS (ESI) m/z calcd for C₃₂H₃₄F₇NO: 651.26 [M+H]⁺; found 651.30. LC-MS: >97% purity.

Table 12 describes compounds prepared following procedures described in Example 11 using appropriate reagents. (Note: Mer IC₅₀: ++++ means <10 nM; +++ means between 10-100 nM, ++ means between 100 nM-1 μM; + means between 1-30 μM; − means inactive.)

TABLE 12

| | | | | | Physical Data |
|---|---|---|---|---|---|
| | | TYRO3 | MERTK | AXL | MS m/z (M + 1) or/and ¹H |
| Compound | Structure | IC₅₀ | IC₅₀ | IC₅₀ | NMR |
| 49 | 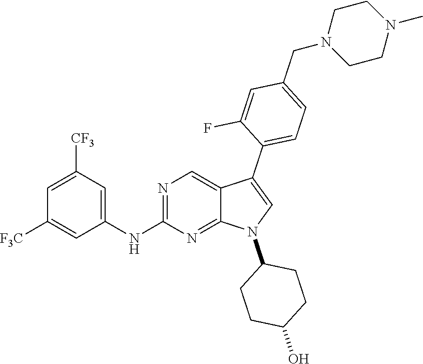 | ++++ | ++ | | ¹H NMR (500 MHz, CD₃OD) δ 9.15 (s, 1H), 8.44 (s, 2H), 8.15 (s, 1H), 7.80-7.73 (m, 2H), 7.71- 7.62 (m, 3H), 4.66 (tt, J = 11.7, 4.1 Hz, 1H), 4.42 (s, 2H), 3.78-3.68 (m, 3H), 3.68-3.47 (m, 6H), 3.00 (s, 3H), 2.20-2.10 (m, 4H), 2.10- 2.02 (m, 2H), 1.54 (qd, J = 12.9, 4.0 Hz, 2H). MS (ESI) m/z calcd for C₃₂H₃₄F₇N₆O: 651.26 M + H]⁺; found 651.30. |
| 50 | 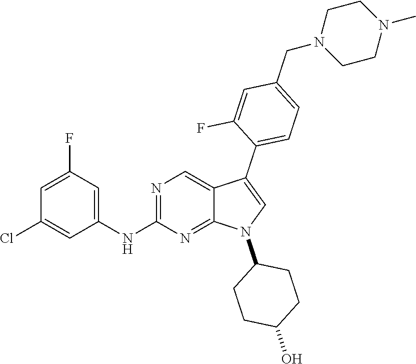 | ++++ | ++++ | | ¹H NMR (500 MHz, CD₃OD) δ 9.08 (d, J = 2.9 Hz, 1H), 8.08 (d, J = 3.8 Hz, 1H), 7.76 (dt, J = 2.5, 1.3 Hz, 1H), 7.72 (t, J = 7.4 Hz, 1H), 7.63 (dd, J = 14.4, 9.9 Hz, 2H), 7.50 (dt, J = 10.7, 2.1 Hz, 1H), 7.03 (dt, J = 8.4, 2.1 Hz, 1H), 4.67- 4.56 (m, 1H), 4.28 (s, 2H), 3.81- 3.73 (m, 1H), 3.69-3.42 (m, 5H), 3.01-2.95 (m, 3H), 2.22- 2.12 (m, 5H), 1.63-1.52 (m, 2H), 1.44-1.27 (m, 4H). MS (ESI) m/z calcd for C₃₀H₃₄ClF₂N₆O⁺: 567.23 [M + H]⁺; found m/z 567.20. |
| 172 | 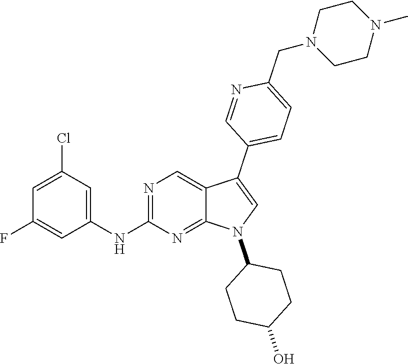 | ++++ | ++++ | | ¹H NMR (500 MHz, CD₃OD) δ 9.20 (s, 1H), 9.15 (s, 1H), 8.80 (d, J = 8.2 Hz, 1H), 8.26 (s, 1H), 8.05 (d, J = 8.1 Hz, 1H), 7.80 (s, 1H), 7.55 (d, J = 10.9 Hz, 1H), 6.96 (d, J = 8.3 Hz, 1H), 4.70-4.61 (m, 1H), 4.21 (s, 2H), 3.82-3.73 (m, 1H), 3.68-3.50 (m, 3H), 3.49- 3.34 (m, 5H), 2.97 (s, 3H), 2.25- 2.10 (m, 4H), 1.66-1.51 (m, 2H), 1.38-1.24 (m, 2H).MS (ESI) m/z calcd for C₂₉H₃₄ClFN₇O⁺: 550.24 [M + H]⁺; found m/z 550.20. |
| 173 | 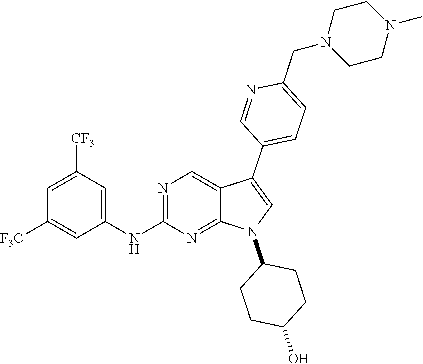 | ++++ | ++ | | ¹H NMR (500 MHz, CD₃OD) δ 9.31 (d, J = 14.4 Hz, 1H), 9.19 (d, J = 5.6 Hz, 1H), 8.82 (d, J = 8.1 Hz, 1H), 8.45 (d, J = 6.1 Hz, 3H), 8.08 (d, J = 8.1 Hz, 1H), 7.75 (d, J = 21.4 Hz, 1H), 4.74-4.64 (m, 1H), 4.27 (s, 2H), 3.79-3.71 (m, 1H), 3.67-3.55 (m, 2H), 3.51- 3.37 (m, 2H), 3.05-2.91 (m, 5H), 2.27-2.02 (m, 6H), 1.63-1.50 (m, 2H). MS (ESI) m/z calcd for C₃₁H₃₄F₆N₇O⁺: 634.27 [M + H]⁺; found m/z 634.30. |

Many modifications and other embodiments of the inventions set forth herein will come to mind to one skilled in the art to which the inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### REFERENCES

1. Jiang, L.; Chen, X. Q.; Gao, M. J.; Lee, W.; Zhou, J.; Zhao, Y. F.; Wang, G. D., The Pros1/Tyro3 axis protects against periodontitis by modulating STAT/SOCS signalling. Journal of cellular and molecular medicine 2019, 23 (4), 2769-2781.
2. Ubil, E.; Caskey, L.; Holtzhausen, A.; Hunter, D.; Story, C.; Earp, H. S., Tumor-secreted Pros1 inhibits macrophage M1 polarization to reduce antitumor immune response. The Journal of clinical investigation 2018, 128 (6), 2356-2369.
3. Berzaghi, R.; Maia, V.; Pereira, F.; Melo, F.; Guedes, M.; Origassa, C.; Scutti, J.; Matsuo, A.; Câmara, N.; Rodrigues, E., SOCS1 favors the epithelial-mesenchymal transition in melanoma, promotes tumor progression and prevents antitumor immunity by PD-L1 expression. Scientific reports 2017, 7, 40585.
4. Holtzhausen, A.; Harris, W.; Ubil, E.; Hunter, D. M.; Zhao, J.; Zhang, Y.; Zhang, D.; Liu, Q.; Wang, X.; Graham, D. K.; Frye, S. V.; Earp, H. S., TAM Family Receptor kinase inhibition reverses MDSC-mediated suppression and augments anti-PD-1 therapy in melanoma. Cancer immunology research 2019, canimm. 0008.2019.
5. Smart, S.; Vasileiadi, E.; Wang, X.; DeRyckere, D.; Graham, D., The Emerging Role of TYRO3 as a Therapeutic Target in Cancer. Cancers 2018, 10 (12), 474.
6. Dufour, F.; Silina, L.; Neyret-Kahn, H.; Moreno-Vega, A.; Krucker, C.; Karboul, N.; Dorland-Galliot, M.; Maillé, P.; Chapeaublanc, E.; Allory, Y., TYRO3 as a molecular target for growth inhibition and apoptosis induction in bladder cancer. Br. J. Cancer 2019, 120, 555-564.
7. Michelson, A. D., Antiplatelet therapies for the treatment of cardiovascular disease. Nat Rev Drug Discov 2010, 9 (2), 154-69.
8. Chapin, J. C.; Hajjar, K. A., Fibrinolysis and the control of blood coagulation. Blood Rev 2015, 29 (1), 17-24.
9. Sakurai, Y.; Hardy, E. T.; Ahn, B.; Tran, R.; Fay, M. E.; Ciciliano, J. C.; Mannino, R. G.; Myers, D. R.; Qiu, Y.; Carden, M. A.; Baldwin, W. H.; Meeks, S. L.; Gilbert, G. E.; Jobe, S. M.; Lam, W. A., A microengineered vascularized bleeding model that integrates the principal components of hemostasis. Nat Commun 2018, 9 (1), 509.
10. Lin, H.; Xu, L.; Yu, S.; Hong, W.; Huang, M.; Xu, P., Therapeutics targeting the fibrinolytic system. Exp Mol Med 2020, 52 (3), 367-379.
11. Palta, S.; Saroa, R.; Palta, A., Overview of the coagulation system. Indian J Anaesth 2014, 58 (5), 515-23.
12. Sasaki, T.; Knyazev, P. G.; Cheburkin, Y.; Gohring, W.; Tisi, D.; Ullrich, A.; Timpl, R.; Hohenester, E., Crystal structure of a C-terminal fragment of growth arrest-specific protein Gas6. Receptor tyrosine kinase activation by laminin G-like domains. J Biol Chem 2002, 277 (46), 44164-70.
13. Virani, S. S.; Alonso, A.; Benjamin, E. J.; Bittencourt, M. S.; Callaway, C. W.; Carson, A. P.; Chamberlain, A. M.; Chang, A. R.; Cheng, S.; Delling, F. N.; Djousse, L.; Elkind, M. S. V.; Ferguson, J. F.; Fornage, M.; Khan, S. S.; Kissela, B. M.; Knutson, K. L.; Kwan, T. W.; Lackland, D. T.; Lewis, T. T.; Lichtman, J. H.; Longenecker, C. T.; Loop, M. S.; Lutsey, P. L.; Martin, S. S.; Matsushita, K.; Moran, A. E.; Mussolino, M. E.; Perak, A. M.; Rosamond, W. D.; Roth, G. A.; Sampson, U. K. A.; Satou, G. M.; Schroeder, E. B.; Shah, S. H.; Shay, C. M.; Spartano, N. L.; Stokes, A.; Tirschwell, D. L.; VanWagner, L. B.; Tsao, C. W.; American Heart Association Council on, E.; Prevention Statistics, C.; Stroke Statistics, S., Heart Disease and Stroke Statistics-2020 Update: A Report From the American Heart Association. Circulation 2020, 141 (9), e139-e596.
14. Kong, D.; Xue, T.; Guo, B.; Cheng, J.; Liu, S.; Wei, J.; Lu, Z.; Liu, H.; Gong, G.; Lan, T.; Hu, W.; Yang, Y., Optimization of P2Y12 Antagonist Ethyl 6-(4-((Benzylsulfonyl) carbamoyl) piperidin-1-yl)-5-cyano-2-methylnicotinate (AZD1283) Led to the Discovery of an Oral Antiplatelet Agent with Improved Druglike Properties. J Med Chem 2019, 62 (6), 3088-3106.
15. Delesque-Touchard, N.; Pflieger, A. M.; Bonnet-Lignon, S.; Millet, L.; Salel, V.; Boldron, C.; Lassalle, G.; Herbert, J. M.; Savi, P.; Bono, F., SAR216471, an alternative to the use of currently available P2Y (1) (2) receptor inhibitors? Thromb Res 2014, 134 (3), 693-703.
16. Dilger, A. K.; Pabbisetty, K. B.; Corte, J. R.; De Lucca, I.; Fang, T.; Yang, W.; Pinto, D. J. P.; Wang, Y.; Zhu, Y.; Mathur, A.; Li, J.; Hou, X.; Smith, D.; Sun, D.; Zhang, H.; Krishnananthan, S.; Wu, D. R.; Myers, J. E., Jr.; Sheriff, S.; Rossi, K. A.; Chacko, S.; Zheng, J. J.; Galella, M. A.; Ziemba, T.; Dierks, E. A.; Bozarth, J. M.; Wu, Y.; Crain, E.; Wong, P. C.; Luettgen, J. M.; Wexler, R. R.; Ewing, W. R., Discovery of Milvexian, a High-Affinity, Orally Bioavailable Inhibitor of Factor XIa in Clinical Studies for Antithrombotic Therapy. J Med Chem 2022, 65 (3), 1770-1785.
17. Ahmed, S.; Gul, S.; Siraj, S.; Hussain, A.; Sheikh, F. S.; Shah, S. U.; Janjua, K.; Khan, H.; Hamdard, M. H., Antiplatelet response to clopidogrel is associated with a haplotype in CYP2C19 gene in Pakistani patients. Sci Rep 2022, 12 (1), 6171.
18. Tibes, R.; Trent, J.; Kurzrock, R., Tyrosine kinase inhibitors and the dawn of molecular cancer therapeutics. Annu Rev Pharmacol Toxicol 2005, 45, 357-84.
19. Powell, N. A.; Hoffman, J. K.; Ciske, F. L.; Kaufman, M. D.; Kohrt, J. T.; Quin, J., 3rd; Sheehan, D. J.; Delaney, A.; Baxi, S. M.; Catana, C.; McConnell, P.; Ohren, J.; Perrin, L. A.; Edmunds, J. J., Highly selective 2,4-diaminopyrimidine-5-carboxamide inhibitors of Sky kinase. Bioorg Med Chem Lett 2013, 23 (4), 1046-50.
20. Powell, N. A.; Hoffman, J. K.; Ciske, F. L.; Kohrt, J. T.; Baxi, S. M.; Peng, Y. W.; Zhong, M.; Catana, C.; Ohren, J.; Perrin, L. A.; Edmunds, J. J., Optimization of highly selective 2,4-diaminopyrimidine-5-carboxamide inhibitors of Sky kinase. Bioorg Med Chem Lett 2013, 23 (4), 1051-5.
21. Cosemans, J., At the MERcy of platelet primers. J Thromb Haemost 2018, 16 (2), 349-351.
22. Angelillo-Scherrer, A.; de Frutos, P.; Aparicio, C.; Melis, E.; Savi, P.; Lupu, F.; Arnout, J.; Dewerchin, M.; Hoylaerts, M.; Herbert, J.; Collen, D.; Dahlback, B.; Carmeliet, P., Deficiency or inhibition of Gas6 causes platelet dysfunction and protects mice against thrombosis. Nat Med 2001, 7 (2), 215-21.
23. Schroeder, G. M.; An, Y.; Cai, Z.-W.; Chen, X.-T.; Clark, C.; Cornelius, L. A.; Dai, J.; Gullo-Brown, J.; Gupta, A.; Henley, B., Discovery of N-(4-(2-amino-3-chloropyridin-4-yloxy)-3-fluorophenyl)-4-ethoxy-1-(4-fluorophenyl)-2-oxo-1,2-dihydropyridine-3-carboxamide (BMS-777607), a selective and orally efficacious inhibitor of the Met kinase superfamily. Journal of medicinal chemistry 2009, 52 (5), 1251-1254.
24. Yokoyama, Y.; Lew, E. D.; Seelige, R.; Tindall, E. A.; Walsh, C.; Fagan, P. C.; Lee, J. Y.; Nevarez, R.; Oh, J.; Tucker, K. D.; Chen, M.; Diliberto, A.; Vaaler, H.; Smith, K. M.; Albert, A.; Li, G.; Bui, J. D., Immuno-Oncological Efficacy of RXDX-106, a Novel Small Molecule Inhibitor of the TAM (TYRO3, AXL, MER) Family of Kinases. Cancer research 2019, 79 (8), 1996-2008.
25. Yokoyama, Y.; Lew, E. D.; Seelige, R.; Walsh, C.; Barrera, M.; Tindall, E.; Oh, J.; Ely, H.; Diliberto, A.; Albert, A., Immuno-oncological efficacy of RXDX-106, a novel, selective and potent small molecule TAM (TYRO3, AXL, MER) inhibitor. AACR: 2017.
26. Powell, N. A.; Hoffman, J. K.; Ciske, F. L.; Kohrt, J. T.; Baxi, S. M.; Peng, Y.-W.; Zhong, M.; Catana, C.; Ohren, J.; Perrin, L. A., Optimization of highly selective 2,4-diaminopyrimidine-5-carboxamide inhibitors of Sky kinase. Bioorganic & medicinal chemistry letters 2013, 23 (4), 1051-1055.
27. Wong, J. P.; Stuhlmiller, T. J.; Giffin, L. C.; Lin, C.; Bigi, R.; Zhao, J.; Zhang, W.; Cruz, A. G. B.; Park, S. I.; Earp, H. S.; Dittmera, D. P.; Frye, S.; Wang, X.; Johnson, G. L.; Damania, B., Kinome profiling of non-Hodgkin lymphoma identifies Tyro3 as a therapeutic target in primary effusion lymphoma. Proceedings of the National Academy of Sciences 2019, 116 (33), 16541-16550.
28. Engelmann, J.; Zarrer, J.; Gensch, V.; Riecken, K.; Berenbrok, N.; Luu, T. V.; Beitzen-Heineke, A.; Vargas-Delgado, M. E.; Pantel, K.; Bokemeyer, C., Regulation of bone homeostasis by MERTK and TYRO3. Nature Communications 2022, 13 (1), 1-15.
29. Gould, W. R.; Baxi, S. M.; Schroeder, R.; Peng, Y. W.; Leadley, R. J.; Peterson, J. T.; Perrin, L. A., Gas6 receptors Axl, Sky and Mer enhance platelet activation and regulate thrombotic responses. Journal of thrombosis and haemostasis: JTH 2005, 3 (4), 733-41.

What is claimed is:

1. A compound of Formula A 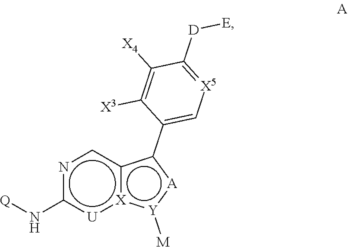
wherein:
U is N or CH; X is N or C; Y is N or C, and A is N or CH, wherein
two or three of U, X, Y and A is N, and at least one of U and X is N;
D is selected from the group consisting of a bond, (CH₂)ₘ, (CD₂)ₘ, (CHD)ₘ, (CF₂)ₘ, (CHF)ₘ, —O—, —CO—, —N(H)— and —S(O₂)—;
E is selected from the group consisting of:
—NR^{E1}R^{E2}, wherein R^{E1} is hydrogen, deuterium or C₁-C₆ alkyl, and R^{E2} is substituted or unsubstituted C₁-C₆ alkyl; and, 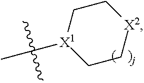
wherein:
X¹ is CH or N;
X² is CH₂, O, S, or N—R⁶, wherein R⁶ is hydrogen, substituted or unsubstituted C₁-C₆ alkyl, or C₃-C₉ cycloalkyl;
X³ and X⁴ are independently selected from hydrogen and halogen;
X⁵ is CH or N;
Q is selected from the group consisting of: 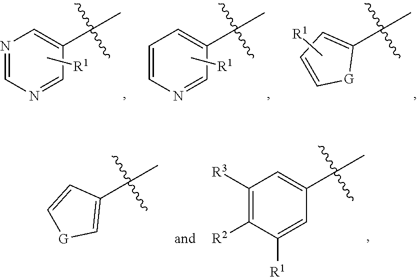
wherein, G is S, O or N;
M is 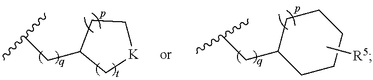
K is O or CR⁷R⁸, wherein R⁷ and R⁸ are independently selected from C₁-C₆ alkylhydroxy, or R⁷ and R⁸ together with the carbon to which each is attached form a carbonyl or a 5-6 membered heterocycloalkyl;
m is 0, 1, or 2;
j is 0, 1, 2, or 3;
p is 0 or 1;
t is 1 or 2;
q is 0 or 1;
R¹ is selected from the group consisting of halogen, C₁-C₆ alkyl, C₁-C₆ alkoxy, hydroxy, nitro, C₁-C₆-haloalkyl, cyano, and N(R^{4a})₂,
wherein each R^{4a} is independently selected from the group consisting of hydrogen, and C₁-C₆-alkyl;
R² and R³ are each independently selected from the group consisting of hydrogen, deuterium, halogen, C₁-C₆ alkyl, C₁-C₆ alkoxy, hydroxy, nitro, C₁-C₆-haloalkyl, cyano, and N(R^{4b})₂,
wherein each R^{4b} is independently selected from the group consisting of hydrogen and C₁-C₆-alkyl;
or,
R¹ and R² or R² and R³ together with the carbon to which each is attached form a ring selected from the group consisting of substituted or unsubstituted C₃-C₈ cycloalkyl, substituted or unsubstituted C₃-C₈ heterocycloalkyl, substituted or unsubstituted C₃-C₈ aryl, and substituted or unsubstituted C₃-C₈ heteroaryl, wherein the heteroatom is O, N, or S; and,
R⁵ is selected from the group consisting of hydroxy, cyano, halogen, amino, C₁-C₆ alkyl, C₁-C₆ alkoxy, mono(C₁-C₆)alkylamino di(C₁-C₆)alkylamino, N-alkoxycarbonyl, and C₁-C₆ alkylhydroxy;
or a pharmaceutically acceptable salt thereof.

2. The compound of claim 1, wherein M is 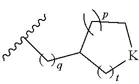
and:
K is CR⁷R⁸, wherein R⁷ and R⁸ are independently selected from C₁-C₆ alkylhydroxy; q is 0; p is 0; and t is 1, the compound having a structure of Formula A-1: 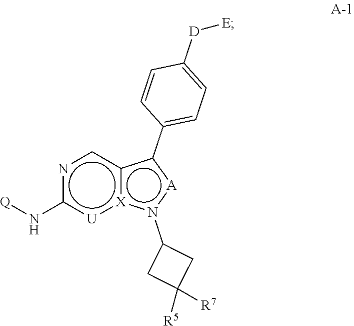
or,
K is O; q is 0; p is 1; and t is 2, the compound having a structure of Formula A-2: 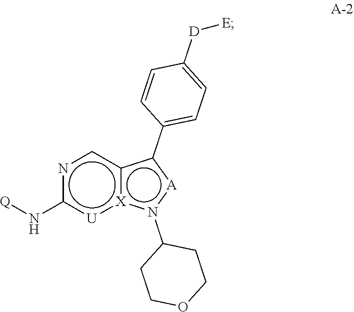
or,
K is CR⁷R⁸, wherein R⁷ and R⁸ together with the carbon to which each is attached form a carbonyl; q is 0; p is 1; and t is 2, the compound having a structure of Formula A-3: 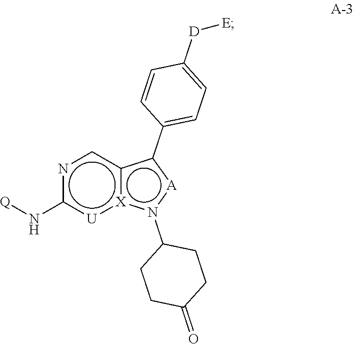
or,
K is CR⁷R⁸, wherein R⁷ and R⁸ together with the carbon to which each is attached form a 5-6 membered heterocycloalkyl; q is 0; p is 1; and t is 2, the compound having a structure of Formula A-4:

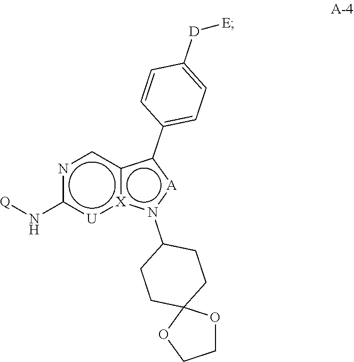

3. The compound of claim 1, wherein:
X³ is H, X⁴ is F, and X⁵ is CH, the compound having a structure of Formula A-5: 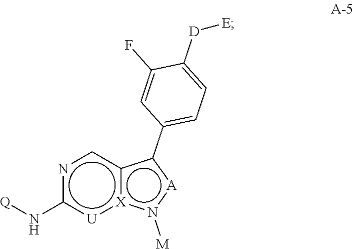
or
X³ is F, X⁴ is H, and X⁵ is CH, the compound having a structure of Formula A-6: 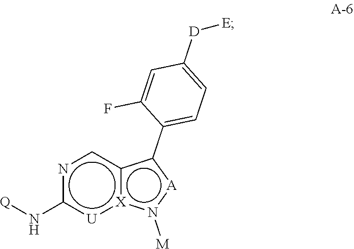
or
X³ is H, X⁴ is H, and X⁵ is N, the compound having a structure of Formula A-7:

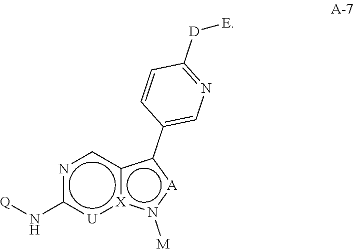

4. The compound of claim 1, wherein:
M is 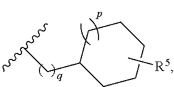
the compound having a structure of Formula I 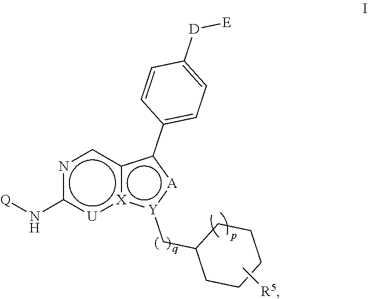
or a pharmaceutically acceptable salt thereof.

5. The compound of claim 4, wherein:
U is N; X is C; Y is N; and A is CH, the compound having a structure of Formula I-1: 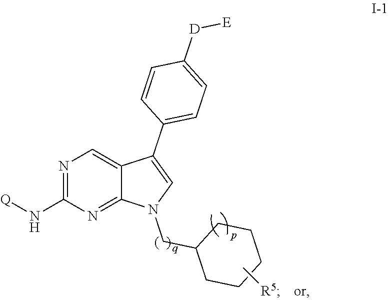
U is N; X is C; Y is N; and A is N, the compound having a structure of Formula I-2: 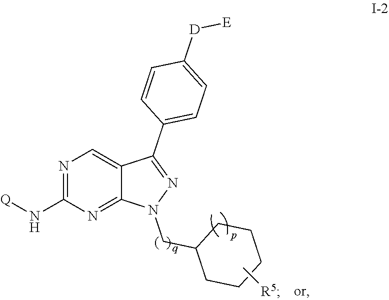
U is N; X is N; Y is C; and A is N, the compound having a structure of Formula I-3: 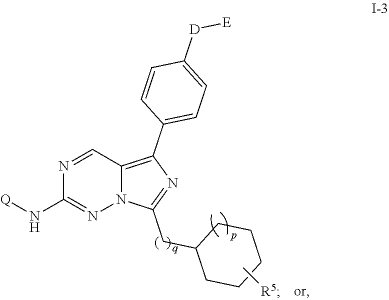
U is N; X is N; Y is C; and A is CH, the compound having a structure of Formula I-4: 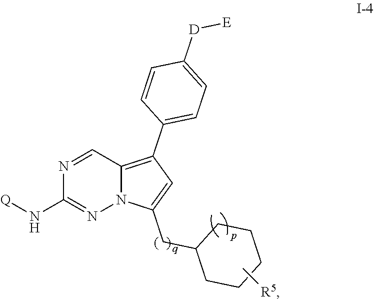
or a pharmaceutically acceptable salt thereof.

6. A compound of claim 5, of Formula I-1 or I-2, and having a structure of Formula Ia: 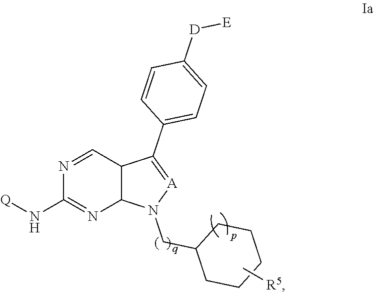
or a pharmaceutically acceptable salt thereof.

7. A compound of claim 5, of Formula I-3, I-4 or I-5, and having a structure of Formula Ib: 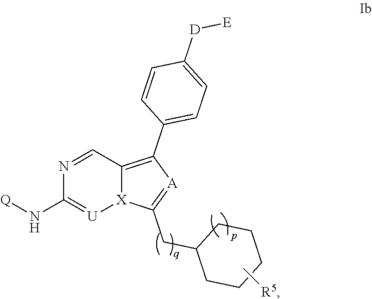
or a pharmaceutically acceptable salt thereof.

8. The compound of any one of claims 1-7, or a pharmaceutically acceptable salt thereof, wherein E is

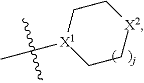

9. The compound of any one of claims 1-8, or a pharmaceutically acceptable salt thereof, wherein Q is selected from the group consisting of:

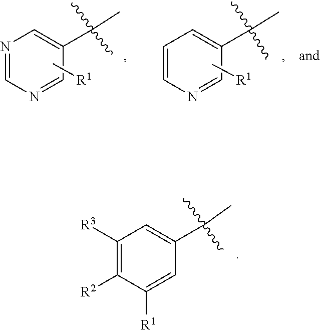

10. The compound of claim 9, or a pharmaceutically acceptable salt thereof, wherein Q is

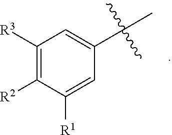

11. The compound of claim 10, or a pharmaceutically acceptable salt thereof, wherein
R¹ is selected from the group consisting of halogen, C₁-C₆ alkyl, C₁-C₆ alkoxy, hydroxy, nitro, C₁-C₆-haloalkyl, cyano, and N(R^{4a})₂,
wherein each R^{4a} is independently selected from the group consisting of hydrogen, and C₁-C₆-alkyl;
R² and R³ are each independently selected from the group consisting of hydrogen, halogen, C₁-C₆ alkyl, C₁-C₆ alkoxy, hydroxy, nitro, C₁-C₆-haloalkyl, cyano, and N(R^{4b})₂, wherein each R^{4b} is independently selected from the group consisting of hydrogen and C₁-C₆-alkyl.

12. The compound of any one of claims 4-6 and 8-11, or a pharmaceutically acceptable salt thereof, having a structure of Formula Ia-1:

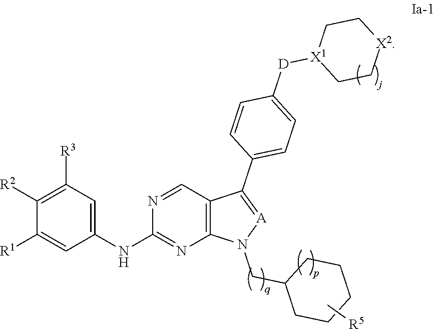

13. The compound of any one of claims 3-4 and 6-10, or a pharmaceutically acceptable salt thereof, having a structure of Formula Ib-1:

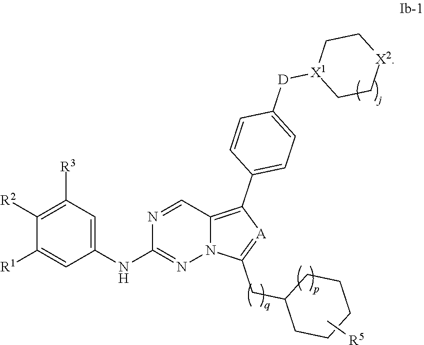

14. The compound of any one of claims 1-13, or a pharmaceutically acceptable salt thereof, wherein q is 0.

15. The compound of any one of claims 1-14, or a pharmaceutically acceptable salt thereof, wherein p is 1.

16. The compound of any one of claims 1-15, or a pharmaceutically acceptable salt thereof, wherein D is selected from the group consisting of (CH₂)ₘ, —O—, —CO—, —N(H)— and —S(O₂)—.

17. The compound of any one of claims 1-16, or a pharmaceutically acceptable salt thereof, wherein D is (CH₂)ₘ, —O— or —N(H)—.

18. The compound of any one of claims 1-17, or a pharmaceutically acceptable salt thereof, wherein R⁵ is selected from the group consisting of hydroxy, halogen, amino, C₁-C₆ alkyl, N-alkoxycarbonyl and C₁-C₆ alkoxy.

19. The compound of claim 18, or a pharmaceutically acceptable salt thereof, wherein R⁵ is selected from the group consisting of hydroxy, amino, methoxy, ethoxy, methyl, NHBoc and ethyl.

20. The compound of claim 19, or a pharmaceutically acceptable salt thereof, wherein R⁵ is hydroxy, methoxy, or ethoxy.

21. The compound of claim 20, or a pharmaceutically acceptable salt thereof, wherein R⁵ is hydroxy.

22. The compound of claim 18, or a pharmaceutically acceptable salt thereof, having a structure of Formula Ia-2:

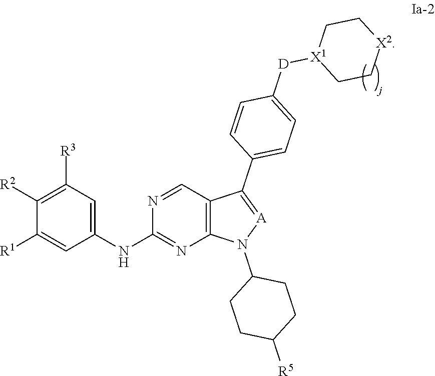

23. The compound of claim 18, or a pharmaceutically acceptable salt thereof, having a structure of Formula Ib-2:

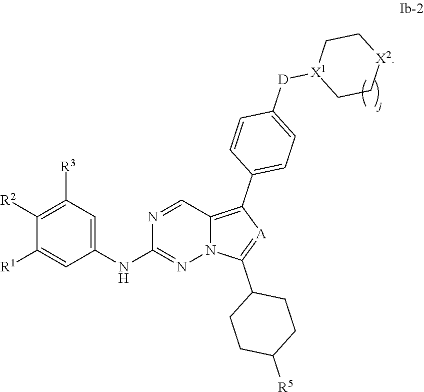

24. The compound of claim 22 or 23, or a pharmaceutically acceptable salt thereof, wherein X¹ is N.

25. The compound of any one of claims 22-24, or a pharmaceutically acceptable salt thereof, wherein X² is S or N—R⁶, wherein R⁶ is hydrogen or a substituted or unsubstituted C₁-C₆ alkyl, or a C₃-C₉ cycloalkyl.

26. The compound of any one of claims 22-25, or a pharmaceutically acceptable salt thereof, wherein X¹ is N, and X² is S or N—R⁶, wherein R⁶ is hydrogen or a substituted or unsubstituted C₁-C₆ alkyl.

27. The compound of any one of claims 22-26, or a pharmaceutically acceptable salt thereof, wherein j is 1 or 2.

28. The compound of any one of claims 1-27, or a pharmaceutically acceptable salt thereof, wherein D is —N(H)— or O.

29. The compound of claim 28, or a pharmaceutically acceptable salt thereof, wherein E is 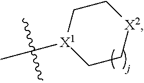
wherein X¹ is CH.

30. The compound of any one of claims 1-27, or a pharmaceutically acceptable salt thereof, wherein D is (CH₂)ₘ.

31. The compound of claim 30, or a pharmaceutically acceptable salt thereof, wherein E is 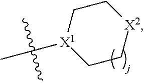
wherein X¹ is N.

32. The compound of any one of claims 22-31, or a pharmaceutically acceptable salt thereof, wherein A is N.

33. The compound of claim 32, or a pharmaceutically acceptable salt thereof, having a structure of Formula Ia-3 or Ib-3:

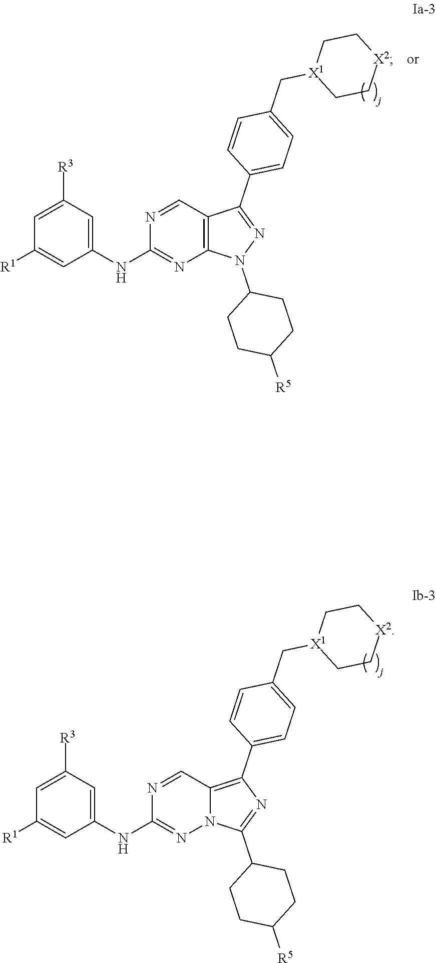

34. The compound of any one of claims 22-31, or a pharmaceutically acceptable salt thereof, wherein A is CH.

35. The compound of claim 34, or a pharmaceutically acceptable salt thereof, having a structure of Formula Ia-4 or Ib-4:

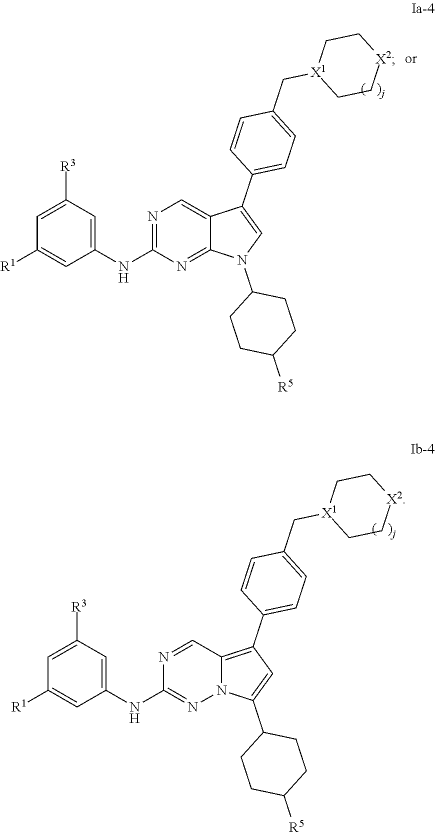

36. The compound of claim 35, or a pharmaceutically acceptable salt thereof, having a structure of Formula Ia-5:

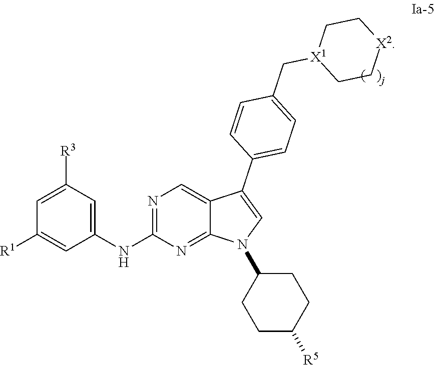

37. The compound of claim 35, or a pharmaceutically acceptable salt thereof, having a structure of Formula Ia-6:

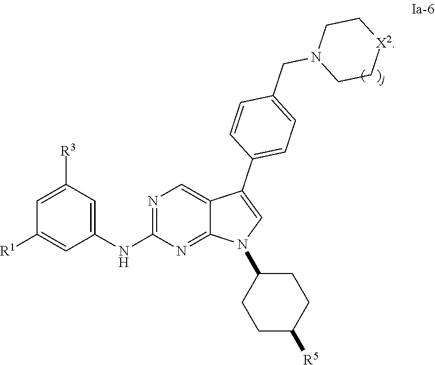

38. The compound of any one of claims 1-4, or a pharmaceutically acceptable salt thereof, wherein D is CH₂; and E is —NR^{E1}R^{E2}, wherein R^{E1} is hydrogen, and R^{E2} is C₁-C₆ alkyl substituted once with mono(C₁-C₆ alkyl)amino, di(C₁-C₆ alkyl)amino, or a 5-7 member heterocyclyl.

39. The compound of claim 4, or a pharmaceutically acceptable salt thereof, having a structure of Formula Ic:

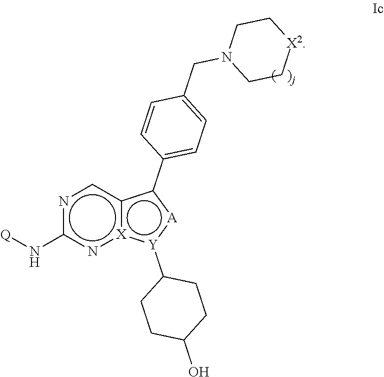

40. The compound of claim 39, or a pharmaceutically acceptable salt thereof, having a structure of Formula Ic-1:

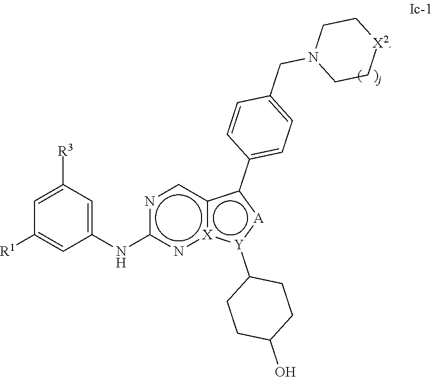

41. The compound of claim 40, or a pharmaceutically acceptable salt thereof, having a structure of Formula Ic-2:

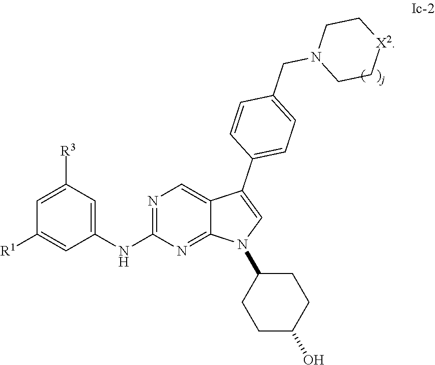

42. The compound of claim 41, or a pharmaceutically acceptable salt thereof, wherein X² is N—R⁶, wherein R⁶ is C₁-C₆ alkyl.

43. The compound of claim 42, or a pharmaceutically acceptable salt thereof, wherein R⁶ is methyl.

44. The compound of any one of claims 39-43, or a pharmaceutically acceptable salt thereof, wherein
R¹ is selected from the group consisting of halogen, C₁-C₆ alkyl, C₁-C₆ haloalkyl and C₁-C₆ alkoxy; and,
R³ is selected from the group consisting of halogen, C₁-C₆ alkyl, C₁-C₆ haloalkyl and C₁-C₆ alkoxy.

45. The compound of claim 44, or a pharmaceutically acceptable salt thereof, wherein
R¹ is selected from the group consisting of fluoro, —CF₃ and methoxy; and,
R³ is selected from the group consisting of chloro, —CF₃ and methoxy.

46. The compound of claim 1 or pharmaceutically acceptable salt thereof, as shown in Table 1, Table 1A, or Table 1B.

47. A pharmaceutical composition comprising a therapeutically effective amount of a compound of any one of claims 1-46, or a pharmaceutically acceptable salt thereof, and a pharmaceutically acceptable carrier.

48. A method for the treatment of a disorder associated with TAM receptor tyrosine kinases, the method comprising the step of administering to the subject an effective amount of a compound of any one of claims 1-46, or the pharmaceutical composition of claim 47.

49. The method of claim 48, wherein the method comprises treatment of a disorder associated with TYRO3 or Mer tyrosine kinases.

50. The method of claim 48, wherein the method comprises treatment of a disorder associated with TYRO3 and Mer tyrosine kinases.

51. The method of claim 48, wherein the method comprises treatment of a disorder associated with TYRO3 tyrosine kinase.

52. The method of any one of claims 48-51, wherein the disorder is a cancer, an infection, a fibrosis, a thrombotic disorder, or a clotting disorder.

53. The method of claim 52, wherein the disorder is a cancer.

54. The method of claim 53, wherein the cancer is selected from the group consisting of breast cancer, cervical cancer, gastrointestinal cancer, colorectal cancer, brain cancer, skin cancer, prostate cancer, ovarian cancer, thyroid cancer, testicular cancer, pancreatic cancer, liver cancer, bladder cancer, hematologic cancer, endometrial cancer, melanoma, glioma, leukemia, lymphoma, chronic myeloproliferative disorder, myelodysplastic syndrome, myeloproliferative neoplasm, plasma cell neoplasm (myeloma).

55. The method of claim 54, wherein the cancer is TYRO3 and/or MerTK +/+.

56. The method of claim 54, wherein the cancer is TYRO3 and/or MerTK −/−.

57. The method of claim 52, wherein the disorder is a thrombotic disorder or clotting disorder.

58. The method of claim 57, wherein the thrombotic disorder or clotting disorder involves ischemic heart disease, stroke, or acute myocardial infarction.

59. The method of claim 52, wherein the disorder is an infection.

60. The method of claim 59, wherein the infection is viral.

61. The method of claim 52, wherein the disorder is fibrosis.

62. The method of claim 48, further comprising administering an additional active agent.

63. A method of inhibiting TAM receptor tyrosine kinase, in at least one cell, the method comprising the step of contacting the at least one cell with an effective amount of any one of claims 1-46, or the pharmaceutical composition of claim 47.

64. The method of claim 63 wherein the method comprises inhibiting TYRO3 or Mer tyrosine kinases.

65. The method of claim 63, wherein the method comprises inhibiting TYRO3 and Mer tyrosine kinases.

66. The method of claim 63, wherein the method comprises inhibiting TYRO3 tyrosine kinase alone.

67. The method of any one of claims 63-66, wherein the cell has been isolated from a mammal prior to the contacting step.
